(19) United States
(12) Patent Application Publication
(10) Pub. No.: US 2011/0092988 A1
(43) Pub. Date: Apr. 21, 2011

(54) Microdevices for Tissue Approximation and Retention, Methods for Using, and Methods for Making

(75) Inventors: Adam L. Cohen, Los Angeles, CA (US); Vacit Arat, La Canada Flintridge, CA (US); Dennis R. Smalley, Newhall, CA (US)

(73) Assignee: Microfabrica Inc.

(21) Appl. No.: 12/909,743

(22) Filed: Oct. 21, 2010

Related U.S. Application Data
(63) Continuation of application No. 11/625,807, filed on Jan. 22, 2007.
Application No. 11/625,807 is a continuation-in-part of application No. 11/598,968, filed on Nov. 14, 2006.
Application No. 11/598,968 is a continuation-in-part of application No. 11/582,049, filed on Oct. 16, 2006, now Pat. No. 7,686,770.
Application No. 11/582,049 is a continuation-in-part of application No. 11/444,999, filed on May 31, 2006, now abandoned.
Application No. 11/444,999 is a continuation-in-part of application No. 10/697,598, filed on Oct. 29, 2003, now abandoned.
Application No. 10/697,598 is a continuation-in-part of application No. 11/591,911, filed on Nov. 1, 2006, now abandoned.
Application No. 11/591,911 is a continuation-in-part of application No. 10/697,598, filed on Oct. 29, 2003, now abandoned.
(60) Provisional application No. 60/761,401, filed on Jan. 20, 2006.
(60) Provisional application No. 60/736,961, filed on Nov. 14, 2005.
(60) Provisional application No. 60/761,401, filed on Jan. 20, 2006.
(60) Provisional application No. 60/726,794, filed on Oct. 14, 2005.
(60) Provisional application No. 60/686,496, filed on May 31, 2005.
(60) Provisional application No. 60/422,007, filed on Oct. 29, 2002.
(60) Provisional application No. 60/732,413, filed on Nov. 1, 2005.
(60) Provisional application No. 60/736,961, filed on Nov. 14, 2005.
(60) Provisional application No. 60/761,401, filed on Jan. 20, 2006.

(51) Int. Cl. A61B 17/10; A61B 17/08

(52) U.S. Cl. 606/142; 606/151

(57) ABSTRACT

Embodiments of invention are directed to micro-scale of mesoscale tissue approximation instruments that may be delivered to the body of a patient during minimally invasive or other surgical procedures. In one group of embodiments, the instrument has an elongated (longitudinal) configuration while with two sets of expandable wings that each have a toggle configuration that can be made to expand when located on opposite sides of a distal tissue region and a proximal tissue region and can then be made to move toward one another to bring the two tissue regions into more a proximal position. In some embodiments, multiple tissue approximation instruments are located within a delivery system for sequential delivery to a patient's body.

### RELATED APPLICATIONS

This application is a continuation of U.S. patent application Ser. No. 11/625,807 (Microfabrica Docket No. P-US171-A-MF), filed Jan. 22, 2007. The '807 application claims benefit of U.S. Provisional Application No. 60/761,401, filed Jan. 20, 2006, and the '807 application is a continuation-in-part of U.S. application Ser. No. 11/598,968 (P-US167-A-MF), filed Nov. 14, 2006; Ser. No. 11/582,049 (P-US164-A-MF), filed Oct. 16, 2006, now U.S. Pat. No. 7,686,770; Ser. No. 11/444,999 (P-US159-A-MF), filed May 31, 2006; and Ser. No. 10/697,598 (P-US083-A-MG), filed Oct. 29, 2003, now abandoned. The '968 application claims benefit to U.S. Provisional Application Nos. 60/736,961, filed Nov. 14, 2005; and 60/761,401, filed Jan. 20, 2006; and the '968 application is a continuation-in-part of U.S. patent application Ser. No. 11/591,911 (P-US165-A-MF), filed Nov. 1, 2006. The '049 application in turn claims the benefit to U.S. Provisional Patent Application No. 60/726,794, filed Oct. 14, 2005. The '999 application claims benefit of U.S. Provisional Patent Application No. 60/686,496, filed May 31, 2005 and the '999 application is a continuation-in-part of U.S. patent application Ser. No. 10/697,598 (P-US083-A-MG), filed Oct. 29, 2003, now abandoned. The '598 application claims benefit of U.S. Provisional Patent Application No. 60/422,007, filed Oct. 29, 2002. The '911 application claims benefit of U.S. Provisional Application Nos. 60/732,413, filed Nov. 1, 2005; 60/736,961, filed Nov. 14, 2006; and 60/761,401, filed Jan. 20, 2006. Each of these applications is hereby incorporated herein by reference as if set forth in full herein.

### FIELD OF THE INVENTION

The present invention relates medical devices and in particular medical devices that can be used for tissue approximation and retention/fixation that may be implemented in a surgical procedure (e.g. a minimally invasive surgical procedure). In some embodiments the device or implement may be formed using a multilayer electrochemical fabrication process (e.g. EFAB™ process).

### BACKGROUND OF THE INVENTION

A technique for forming three-dimensional structures (e.g. parts, components, devices, and the like) from a plurality of adhered layers was invented by Adam L. Cohen and is known as Electrochemical Fabrication. It is being commercially pursued by Microfabrica Inc. (formerly MEMGen® Corporation) of Burbank, Calif. under the name EFAB™. This technique was described in U.S. Pat. No. 6,027,630, issued on Feb. 22, 2000. This electrochemical deposition technique allows the selective deposition of a material using a unique masking technique that involves the use of a mask that includes patterned conformable material on a support structure that is independent of the substrate onto which plating will occur. When desiring to perform an electrodeposition using the mask, the conformable portion of the mask is brought into contact with a substrate while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. (formerly MEMGen® Corporation) of Burbank, Calif. such masks have come to be known as INSTANT MASKS™ and the process known as INSTANT MASKING™ or INSTANT MASK™ plating. Selective depositions using conformable contact mask plating may be used to form single layers of material or may be used to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.
(3) A. Cohen, “3-D Micromachining by Electrochemical Fabrication”, Micromachine Devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST '99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFAB™)”, Chapter 19 of The MEMS Handbook, edited by Mohamed Gad-El-Hak, CRC Press, 2002.
(9) Microfabrication—Rapid Prototyping's Killer Application“, pages 1-5 of the Rapid Prototyping Report, CAD/CAM Publishing, Inc., June 1999.

The disclosures of these nine publications are hereby incorporated herein by reference as if set forth in full herein.

The electrochemical deposition process may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate.
2. Then, blanket depositing at least one additional material by electrodeposition so that the additional deposit covers both the regions that were previously selectively deposited onto, and the regions of the substrate that did not receive any previously applied selective depositions.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to the immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated. At least one CC mask is needed for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of the substrate (or onto a previously formed layer or onto a previously deposited portion of a layer) on which deposition is to occur. The pressing together of the CC mask and substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. FIG. 1A also depicts a substrate 6 separated from mask 8. One is as a supporting material for the patterned insulator 10 to maintain its integrity and alignment since the pattern may be topologically complex (e.g., involving isolated “islands” of insulator material). The other function is as an anode for the electroplating operation. CC mask plating selectively deposits material 22 onto a substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C. The CC mask plating process is distinct from a “through-mask” plating process in that in a through-mask plating process the separation of the masking material from the substrate would occur destructively. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1F. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the fabrication of the substrate on which plating is to occur (e.g. separate from a three-dimensional (3D) structure that is being formed). CC masks may be formed in a variety of ways, for example, a photolithographic process may be used. All masks can be generated simultaneously, prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the cathode 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which the feet 68 of subsystem 34 may sit. Subsystem 38 also includes appropriate electrical connections (not shown) for connecting the anode to an appropriate power supply for driving the blanket deposition process.

The planarization subsystem 40 is shown in the lower portion of FIG. 3C and includes a lapping plate 52 and associated motion and control systems (not shown) for planarizing the depositions.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing mask exposures. A first layer of a primary metal is electroplated onto an exposed plating base to fill a void in a photoresist, the photoresist is then removed and a secondary metal is electroplated over the first layer and over the plating base. The exposed surface of the secondary metal is then machined down to a height which exposes the first metal to produce a flat uniform surface extending across the both the primary and secondary metals. Formation of a second layer may then begin by applying a photoresist layer over the first layer and then repeating the process used to produce the first layer. The process is then repeated until the entire structure is formed and the secondary metal is removed by etching. The photoresist is formed over the plating base or previous layer by casting and the voids in the photoresist are formed by exposure of the photoresist through a patterned mask via X-rays or UV radiation.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, and/or more independence between geometric configuration and the selected fabrication process. A need also exists in the field of miniature (i.e. mesoscale and microscale) device fabrication for improved fabrication methods and apparatus.

### SUMMARY OF THE INVENTION

It is an object of some aspects of the invention to provide improved micro or mesoscale medical implements, tools, or instruments.

It is an object of some aspects of the invention to provide improved micro or mesoscale implements, tools, or instruments that may be put in place using minimally invasive surgery and/or that may be useful in performing minimally invasive surgery.

It is an object of some aspects of the invention to provide micro or mesoscale implements, tools, or instruments for minimally invasive surgery where interactive portions of the tool or instrument are extended from a distal end of a housing that is inserted into a body of a patient undergoing surgery.

It is an object of some aspects of the invention to provide micro or mesoscale implements, tools, or instruments that may be used to approximate tissue during a minimally invasive or other surgical procedure.

It is an object of other aspects of the invention to provide methods for fabricating implements, tools, or instruments for use according to the above noted objects of the invention or according to other objects of the invention.

Other objects and advantages of various aspects and embodiments of the invention will be apparent to those of skill in the art upon review of the teachings herein. The various aspects of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single aspect of the invention even though that may be the case with regard to some aspects.

A first aspect of the invention provides a medical instrument for approximating tissue within a patient's body during a minimally invasive surgical procedure, including: (a) a first set of expandable elements; (b) a second set of expandable elements; (c) an elongated structure along which the first and second sets of expandable elements are located; and (d) a locking mechanism for allowing the first and second sets of expandable elements to be moved to a more proximal position, relative to one another, while inhibiting movement of the first and second sets of expandable elements to a more distal positions relative to one another, along the length of the elongated element, after being moved to a more proximal position.

A second aspect of the invention provides a surgical procedure for approximating tissue within a patient's body, including: (a) locating an approximation instrument within the body of a patent at the end of a catheter; the instrument including: (i) a first set of expandable elements located near a distal end of the instrument; (ii) a second set of expandable elements located near a proximal end of the instrument; (iii) an elongated element along which the first and second sets of expandable elements are located; and (IV) a locking mechanism for allowing the first and second sets of expandable elements to be moved to a more proximal position while inhibiting movement of the first and second sets of expandable elements to a more distal position, along the length of the elongated element, after being moved to a more proximal position; (b) inserting a distal end of the instrument through a proximal tissue region and then through a separated distal tissue region; (c) expanding the first set of expandable elements and locating the elements against a wall of the distal tissue region; (d) expanding the second set of expandable elements and locating the elements against a wall of the proximal tissue region; (e) relatively moving the first set of expanded elements and the second set of expandable elements toward one another to bring the proximal and distal tissue regions into a more proximate position; and (f)releasing at least a portion of the instrument from the catheter so that it remain in the body of the patient and retain the distal and proximal tissue regions in the more proximate position.

A third aspect of the invention provides a medical instrument for approximating tissue within a patient's body during a minimally invasive surgical procedure, including (a) a first expandable element; (b) a second expandable element; (c) an elongated element along which the first and second expandable elements are located and separated one from the other; (d) a mechanism for causing at least partial expansion of the first expandable element; (e) mechanism for causing at least partial expansion of the second expandable element; and (f) locking mechanism for allowing the first and second expandable elements to be moved to a more proximal position while inhibiting movement of the first and second sets of expandable elements to a more distal position, along the length of the elongated element, after being moved to a more proximal position.

A fourth aspect of the invention provides a medical instrument for approximating two tissue elements within a patient's body during a minimally invasive surgical procedure, including: (a) a first half of a clip; (b) a second half of the clip configured to engage the first half of the clip such that the two half when engaged can effectively hold two tissue elements in an approximated position; (c) a first handle for holding the first half clip; (d) a second handle for holding the second half clip; (e) a pivotable element for holding the first and second handles in a desired position rotatable relative to one another and which upon rotation can be made to cause the first and second half clips to engage one another; (f) a mechanism for separating the first and second half clips when engaged to one another from the first and second handles.

Other aspects of the invention will be understood by those of skill in the art upon review of the teachings herein. These other aspects of the invention may provide various combinations of the aspects presented above as well as provide other configurations, structures, functional relationships, and processes that have not been specifically set forth above.

### BRIEF DESCRIPTION OF THE DRAWINGS

FIGS. 1A-1C schematically depict side views of various stages of a CC mask plating process, while FIGS. 1D-1G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4I schematically depict the formation of a first layer of a structure using adhered mask plating where the blanket deposition of a second material overlays both the openings between deposition locations of a first material and the first material itself.

FIG. 5 provides a perspective overview of a device or implement according to a first group of embodiments of the invention.

FIGS. 6 and 7 provide perspective and side views of the proximal end of the device of FIG. 5.

FIGS. 8 and 9 provide different perspective views of the distal end of the device of FIG. 5.

FIG. 10 depicts proximal and distal tissue walls or elements that are to be approximated.

FIG. 11 illustrates a delivery needle perforating the proximal and distal tissue elements of FIG. 10.

FIG. 12 provides a partially transparent view of the elements of FIG. 11.

FIG. 13 shows some elements of the delivery system in the region of the proximal end of the device of FIG. 5 prior to delivery of the device but after insertion of the needle into the tissue to be approximated.

FIG. 14 provides a sectional view of the elements of FIG. 11

FIG. 15 provides a sectional view of the distal end of the device of FIG. 5 while located within the needle.

FIG. 16 provides a sectional view of the proximal end of the device of FIG. 5 while located within the needle.

FIGS. 17 and 18 provide two different perspective views of the distal end of the device after it has been delivered from the end of the needle and after the wings have partially opened.

FIG. 19 provides a side view while FIG. 20 provides a perspective view of the device and delivery system after the needle has been sufficient withdrawn to allow the proximal wings to leave the needle and partially open.

FIG. 21 provides a perspective view of the state of the delivery process after the device has been pulled back to cause the distal wings to impinge against the distal surface of the distal tissue wall and to become fully opened.

FIG. 22 provides a close up perspective view of the distal wings against the distal side of the distal tissue wall.

FIG. 23 provides a perspective view of the state of the delivery process after the push tube has been pushed or the pull wire has been pulled, or both, to cause the proximal wings to impinge against the proximal surface of the proximal tissue wall and to become fully opened.

FIG. 24 provides a close up perspective view of the proximal wings against the proximal surface of the proximal tissue wall.

FIG. 25 provides a perspective view of the state of the process after the wire has been pulled relative to the push tube such that proximal and distal tissue walls have been brought into a desired relationship (e.g. made to contact).

FIG. 26, like FIG. 25, shows the needle withdrawn from the device such that the junction between the rail puller and the rail may be seen.

FIGS. 27 and 28 provide perspective views of the interface region between the rail and rail puller of the device of FIG. 5 from opposite sides and with a rotation.

FIG. 29 provides a perspective cut view of the interface region between the rail and rail puller of the device of FIG. 5 so that the engagement of the puller and the rail can be seen.

FIG. 30 provides an alternative perspective view of the interface region between the rail and rail puller of the device of FIG. 5.

FIGS. 31 and 32 provide a close up view and a more global view, respectively, of the device of FIG. 5 after it is separated from the delivery system as a result of a relative rotation between the rail and rail puller.

FIGS. 33 and 34 provide additional perspective views of the device of FIG. 5 after it is approximates and retains the distal and proximal tissue walls and after it is disengaged from the delivery system.

FIGS. 35 and 36 provide perspective view of the wide and narrow wings, respectively.

FIGS. 37 and 38 provide perspective view of pairs of wings (partially opened in the case of FIG. 37 and fully opened in the case of FIG. 38) located with respect to each other so that they can share common pivot elements

FIGS. 39 and 40 provide expanded perspective views of the proximal and distal ends of the device of FIG. 5 with the wings removed so that underlying elements, including spring elements may be seen.

FIG. 41 provides an even more expanded view of the distal wing pivots and spring elements.

FIG. 42 provides another perspective view of the distal portion of the device such that the engagement between spring tips and wings can be seen.

FIG. 43 provides an even more expanded view of one of the distal elements.

FIG. 44 provides another perspective view of the proximal portion of the device such that the engagement between a spring tip and a wings can be seen.

FIG. 45 provides another perspective view of the distal end of the device of FIG. 5 showing that the wings while in their fully extended state can be positioned at non-perpendicular angles relative to the longitudinal axis of the device so that seating against a tissue wall can occur at any of a variety of angles.

FIG. 46 shows a sectional close-up of the toothed rail of the device of FIG. 5.

FIG. 47 provides a sectional, perspective view of the rail with one of the crossbars removed, providing a better view of the teeth.

FIG. 48 provides a sectional perspective view of the proximal end of the device with wings removed, the rail removed and the rail puller removed.

FIG. 49 provides an end-on view of the proximal end of the device of FIG. 5 (with wings in the closed position).

FIG. 50 provides a sectional perspective view similar to that of FIG. 48 with the exception that the rail and rail puller have been added back in.

FIG. 51 is provides an end view similar to that of FIG. 49 but with the rail added back in.

FIG. 52 provides a plan view of the catch housing of the device of FIG. 5 with the cover of the catch housing removed so that various components may be seen.

FIG. 53 provides perspective view of the proximal end of the catch housing of the device of FIG. 5 with the cover of the catch housing removed so that various components may be seen.

FIG. 54 provides another plan view of a portion of the catch housing and rail of the device of FIG. 5 so that the re-entrant angle of the teeth of the rail and catch heads may be seen.

FIG. 55 provides a side view of the components of the delivery system relative to a reference 302 (e.g., a port in the patient's body).

FIGS. 56-62 provide side view of depicting various motions of the proximal ends associated with a device delivery process.

FIGS. 63 and 64 depict potential problems with performing a PFO via access through the inferior vena cava while FIG. 65 depict a more preferred approach via access through the superior vena cava.

FIGS. 66A and 66B provide side view of an alternative mechanism for connecting the rail puller and the rail together.

FIGS. 67A-67C provide side views illustrating three alternative tissue anchor mechanisms that can engage issue when inserted into an opening in the tissue.

FIGS. 67D and 67E provide side views of the shoes of FIGS. 67A-67C with alternative surface textures.

FIGS. 68 and 69 depict closed and open configurations of an alternative wing design that open and/or close via rotation about an axis that is parallel to the longitudinal axis of the instrument.

FIG. 70 provides a plan view of a tissue approximation device according to another embodiment of the invention.

FIGS. 71A and 71B provide a top view and a side view of a rail puller useable with the device of FIG. 70.

FIG. 72 provides a perspective view of the tip of an approximation device according to another embodiment of the invention where the tip is sharp enough to penetrate body tissue without the use of a delivery needle.

FIG. 73 provides a schematic illustration of cleet based retention mechanism that may be used in various embodiments of the invention.

FIG. 74 provides a schematic illustration of a rack and pinion based mechanism that can be used to force open the wings of an approximation device according to some alternative embodiments of the invention.

FIGS. 75-81 provide schematic side views of an approximation device delivery system according to another embodiment of the invention at various stages of a delivery and approximation process where the system includes a plurality of approximation devices loaded within the body of a delivery needle which devices may be delivered in sequence to the body of a patient.

FIGS. 82-85 provide schematic side views of a approximation device delivery system according to another embodiment of the invention at various stages of a delivery and approximation process where the system includes a magazine for holding extra devices that are to be delivered.

FIG. 86 depicts an opening between the sides of two tissue elements.

FIG. 87 depict and alternative instrument having a flexible rail that may be useful for closing a side-by-side gap in tissue elements as seen in FIG. 86.

FIGS. 88-91 depict various stages in a embodiment to close the side-by-side gap in tissue elements as seen in FIG. 86.

FIGS. 92-100 provide plan views illustrating an edge-to-edge approximation device having two winged ends connected by a chain and a illustrating a series of steps that may be involved in the delivery of such a device.

FIGS. 101-103 illustrate a method of closing a large opening using a plurality of devices like that of FIGS. 92-100 where each device is delivered and then the ends of the individual devices are brought closer together (by pulling the chain) in a progressive and possibly alternating manner so as to minimize the stress induced in the tissue around any individual device.

FIGS. 104-151 provide various views of a tissue approximating clip device and delivery system according to a fourth group of embodiments of the invention.

FIG. 152 provides a perspective view of three pairs of half clips similar to those in FIGS. 104-151 and a handling frame to which they are attached during formation.

### DETAILED DESCRIPTION OF PREFERRED EMBODIMENTS OF THE INVENTION

Fabrication Methods

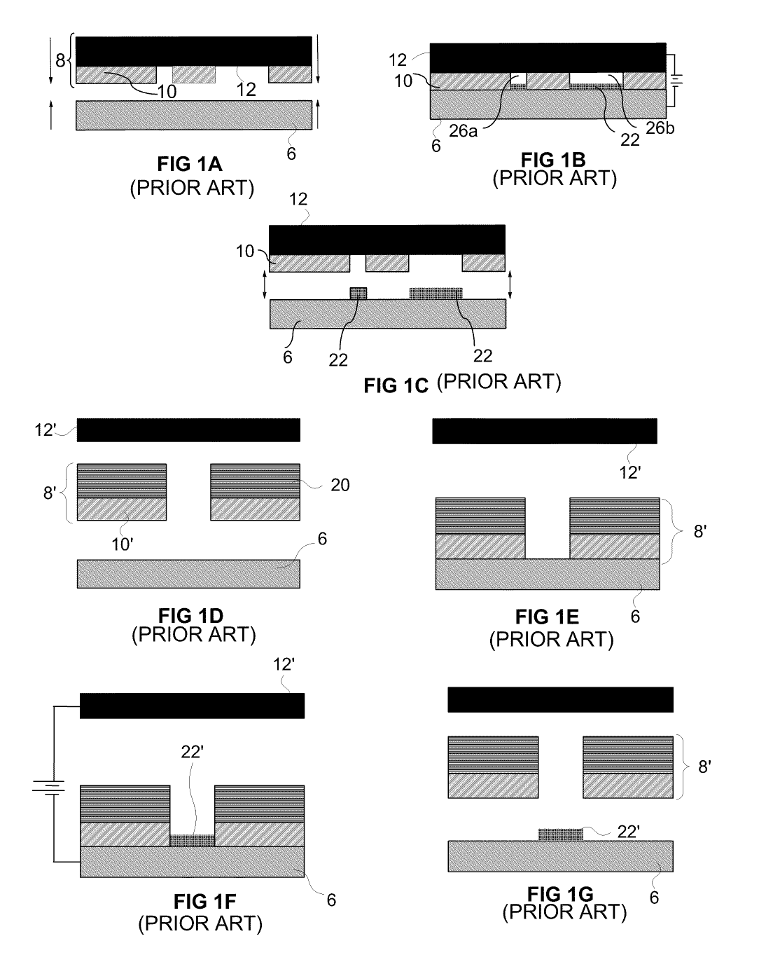

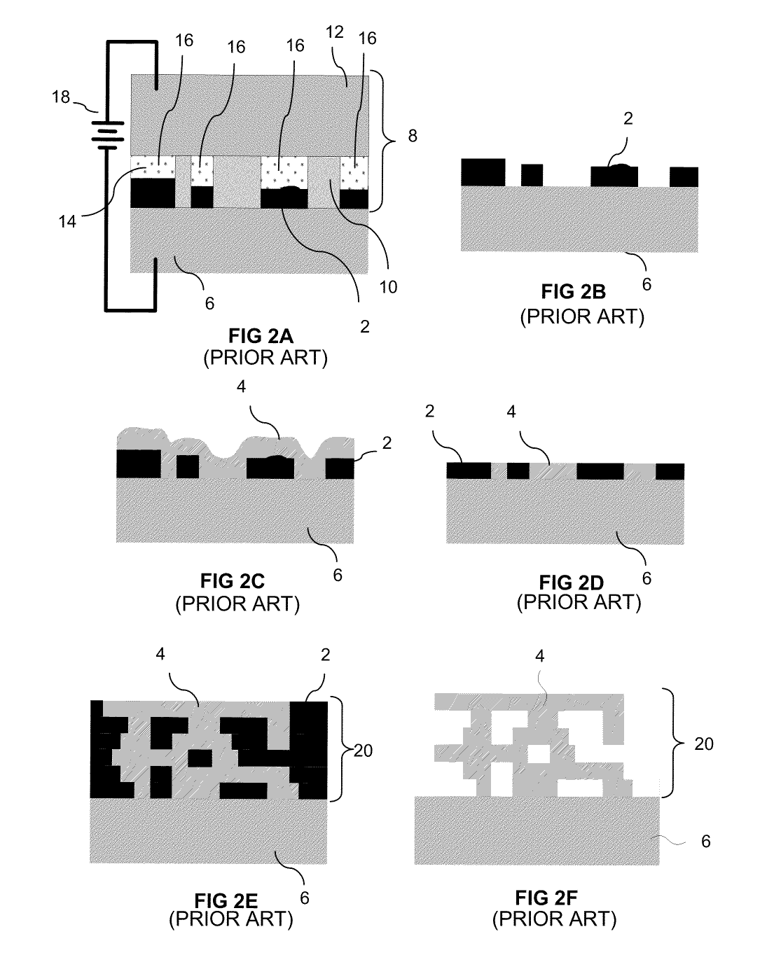

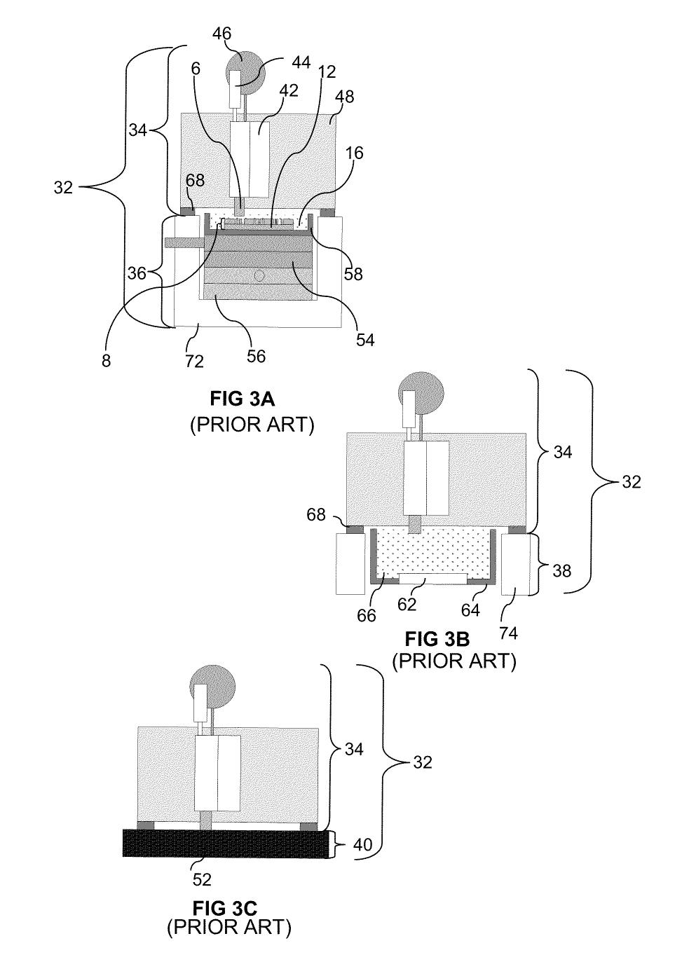

FIGS. 1A-1G, 2A-2F, and 3A-3C illustrate various features of one form of electrochemical fabrication that are known. Other electrochemical fabrication techniques are set forth in the '630 patent referenced above, in the various previously incorporated publications, in various other patents and patent applications incorporated herein by reference, still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

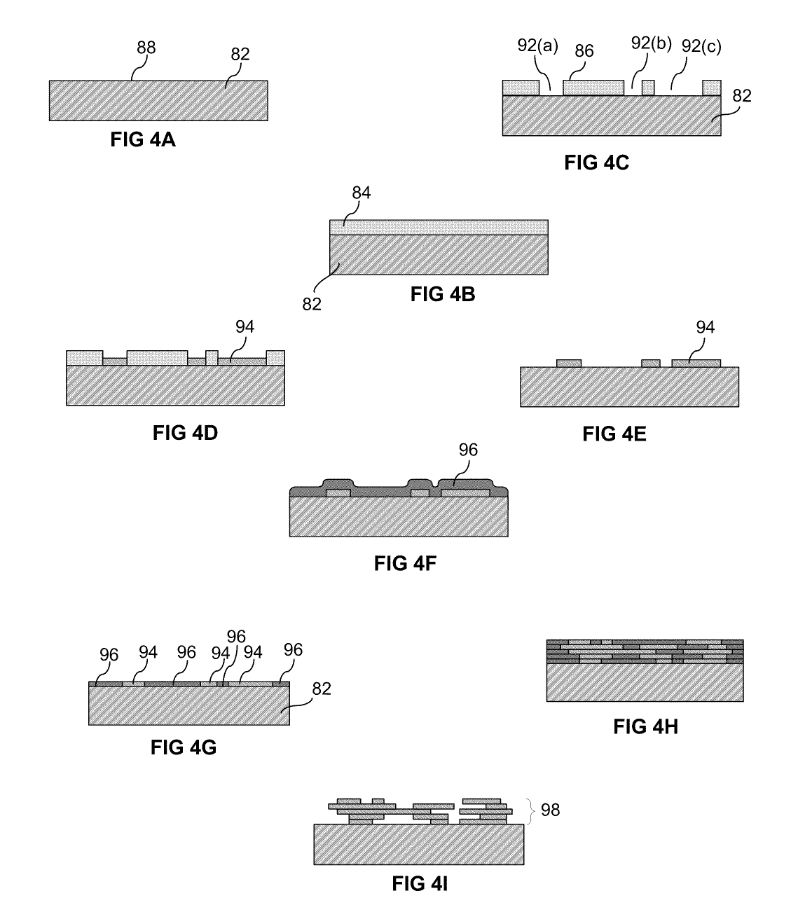

FIGS. 4A-4I illustrate various stages in the formation of a single layer of a multi-layer fabrication process where a second metal is deposited on a first metal as well as in openings in the first metal where its deposition forms part of the layer. In FIG. 4A, a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D, a metal 94 (e.g. nickel) is shown as having been electroplated into the openings 92(a)-92(c). In FIG. 4E, the photoresist has been removed (i.e. chemically stripped) from the substrate to expose regions of the substrate 82 which are not covered with the first metal 94. In FIG. 4F, a second metal 96 (e.g., silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single layer of one or more deposited materials while others are formed from a plurality of layers of deposited materials (e.g. 2 or more layers, more preferably five or more layers, and most preferably ten or more layers). In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations, proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Adhered mask may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels. Such use of selective etching and interlaced material deposited in association with multiple layers is described in U.S. patent application Ser. No. 10/434,519, by Smalley, and entitled “Methods of and Apparatus for Electrochemically Fabricating Structures Via Interlaced Layers or Via Selective Etching and Filling of Voids” which is hereby incorporated herein by reference as if set forth in full.

Building techniques may include the use of more then one planarization operation per layer and in some cases no planarization operations may be used on some layers. Deposition operations may be of the selective and/or blanket type. Selective patterning may be performed by selective etching operations (i.e. etching with a mask applied to control etching locations) and/or blanket etching operations (i.e. etching without a mask in place where patterned etching of selected materials may occur based on susceptibly of different materials to the type of etching operation used and the etchant used). Depositions may include electroplating operations, electrophoretic deposition operations, electroless plating operations, various physical and chemical vapor deposition operations (e.g. sputtering), thermal spray metal deposition operations, and the like. Materials deposited may be conductive, semiconductive, or dielectric. Alternative deposition techniques may include flowing over, spreading, spraying, ink jet dispensing, and the like. Sacrificial materials may be separable from structural materials by selective chemical etching operations, planarization operations, melting operations, and the like. Temporary substrates on which structures may be formed may be of the sacrificial-type (i.e. destroyed or damaged during separation of deposited materials to the extent they can not be reused), non-sacrificial-type (i.e. not destroyed or excessively damaged, i.e. damaged to the extent they may not be reused, with a sacrificial or release layer located between the substrate and the initial layers of a structure that is formed. Non-sacrificial substrates may be considered reusable, with little or no rework (e.g. replanarizing one or more selected surfaces or applying a release layer, and the like) though they may or may not be reused for a variety of reasons.

In some embodiments the formation of the implements, tools, or instruments may include various post layer formation operations. Some such post layer formation operations may include transferring the device from a temporary substrate to another substrate. Some embodiments may employ diffusion bonding or the like to enhance adhesion between successive layers of material. Various teachings concerning the use of diffusion bonding in electrochemical fabrication process is set forth in U.S. Patent Application No. 60/534,204 which was filed Dec. 31, 2003 by Cohen et al. which is entitled “Method for Fabricating Three-Dimensional Structures Including Surface Treatment of a First Material in Preparation for Deposition of a Second Material”; U.S. patent application Ser. No. 10/841,382, filed May 7, 2004 by Zhang, et al., and which is entitled “Method of Electrochemically Fabricating Multilayer Structures Having Improved Interlayer Adhesion”; U.S. patent application Ser. No. 10/841,384, filed May 7, 2004 by Zhang, et al., and which is entitled “Method of Electrochemically Fabricating Multilayer Structures Having Improved Interlayer Adhesion”. Each of these applications is incorporated herein by reference as if set forth in full.

The formation of implements, tools, or instruments may involve a use of structural or sacrificial dielectric materials which may be incorporated into embodiments of the present invention in a variety of different ways. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., and which is entitled “Electrochemical Fabrication Methods Incorporating Dielectric Materials and/or Using Dielectric Substrates”. These patent filings are each hereby incorporated herein by reference as if set forth in full herein.

Further teachings about planarizing layers and setting layers thicknesses and the like are set forth in the following U.S. patent applications which were filed Dec. 31, 2003: (1) U.S. Patent Application No. 60/534,159 by Cohen et al. and which is entitled “Electrochemical

Fabrication Methods for Producing Multilayer Structures Including the use of Diamond Machining in the Planarization of Deposits of Material” and (2) U.S. Patent Application No. 60/534,183 by Cohen et al. and which is entitled “Method and Apparatus for Maintaining Parallelism of Layers and/or Achieving Desired Thicknesses of Layers During the Electrochemical Fabrication of Structures”. An additional filings providing teachings related to planarization are found in U.S. patent application Ser. No. 11/029,220, filed Jan. 3, 2005 by Frodis, et al., and which is entitled “Method and Apparatus for Maintaining Parallelism of Layers and/or Achieving Desired Thicknesses of Layers During the Electrochemical Fabrication of Structures”. These patent filings are each hereby incorporated herein by reference as if set forth in full herein.

Instruments/Devices:

Tissue approximation devices (which remain in the patient's body) and delivery systems for the devices (which do not remain in the patient's body) are both described herein.

The function of tissue approximation and retention is normally performed by sutures, surgical staples, and in some cases, surgical clips. The microtoggle device of some embodiments of the invention have multiple applications in surgery, particularly for minimally-invasive and/or time-sensitive procedures. Compared with suturing and stapling, the device allows approximation and retention to be accomplished within the body (in some cases, within organs and vessels) with only a small perforation or incision required. If desired, approximation and retention can be performed at a site that is a large distance (e.g., 1 meter) from the port used to introduce the device into the body. Moreover, compared with suturing the device allows approximation and retention to be performed much more quickly and easily (e.g., by pushing and pulling on tubes and wires), with a high degree of automation possible. An example of an application for the device is closure of a patent foramen ovale (PFO), a congenital heart condition associated with certain strokes and potentially with a large percentage of migraine headaches. In PFO closure, the objective is to bring together two septa in the heart: the septum primum and septum secundum, which overlap somewhat. Several devices have been developed for PFO closure (e.g., the Premere PFO Closure System of St. Jude Medical, the Amplatzer PFO Occluder of AGA Medical, and the STARFlex Septal Occluder of Nitinol Medical Technologies). All of these devices tend to be very large, which increases the risk of thrombus formation, which on the left side of the heart may produce strokes or other complications. Use of such devices requires the administration of blood thinners which can have adverse side effects. The devices and methods of the present invention may allow the standard open heart surgery approach to be replaced with a less invasive and less risky approach to repairing the PFO and other problems. Another device, used for tissue fastening, may or may not have application for PFO closure and is described in WO 2005/065412 A2, by Kagen et al., assigned to Valentx (Hopkins, Minn.). This device consists of a suture-like element with proximal and distal tabs which can swivel, delivered using a hollow needle. Among the anticipated issues in deploying such a device is the difficulty in rotating the tabs and disengaging the delivery system. Moreover, reliability may be an issue, both in deployment, and in long-term behavior: the tab might swivel back to a position that allows it to pass through the hole in the tissue.

By way of example, approximation and retention of tissue of the sort encountered in closure of a PFO will be assumed in some of the following descriptions of exemplary devices.

In brief, a device according to a first group of embodiments has two pair of pivoting wings which can spread apart, once the device has been delivered through a hollow needle (i.e., a cannula with a sharpened end), to anchor the device. One set of wings is at the distal end of a toothed rail, while the other is at the proximal end of a ratcheting mechanism through which the toothed rail passes and which catches the teeth on the rail to maintain the device in a shortened configuration. The wings of the first device pivot open along an axis that is perpendicular to the longitudinal axis of the device prior to deployment. This first exemplary device may be considered a microtoggle instrument. Various alternative configurations of the first exemplary device are also discussed. In some variations of the first exemplary device, a flexible or curved rail is used to bridge winged elements. In other variations instead of the device having wings that rotate open along one or more axes that are perpendicular to the longitudinal axis of the device, wings that rotate open along one or more axes that are parallel to the longitudinal axis of the device are provided. In still other variations the carrier of the distal pair of wings may have a sharpened tip so that it may self puncture a wall to be anchored. In still other variations instead of using a notch rails to connect the two pairs of winged elements, a cleet mechanism may be provided that normally allows one directional motion. In still other variations, the catches in the retention housing and the notches in the rail may be reversed. In sill further variations, for example, the wings may be forced open by a rack and pinion mechanism instead of by compressed springs. Other variations are possible, some of which are described hereafter. notches in the rail that connects the pairs of wings may be replect

A second exemplary device and various alternatives are also discussed. This second exemplary device also includes wings that pivot outward from the main body of the device but in this embodiment, the wings pivot outward from one or more axes that are parallel to the longitudinal axis of the device.

First Group of Embodiments—Front to Back Approximation with Winged Devices:

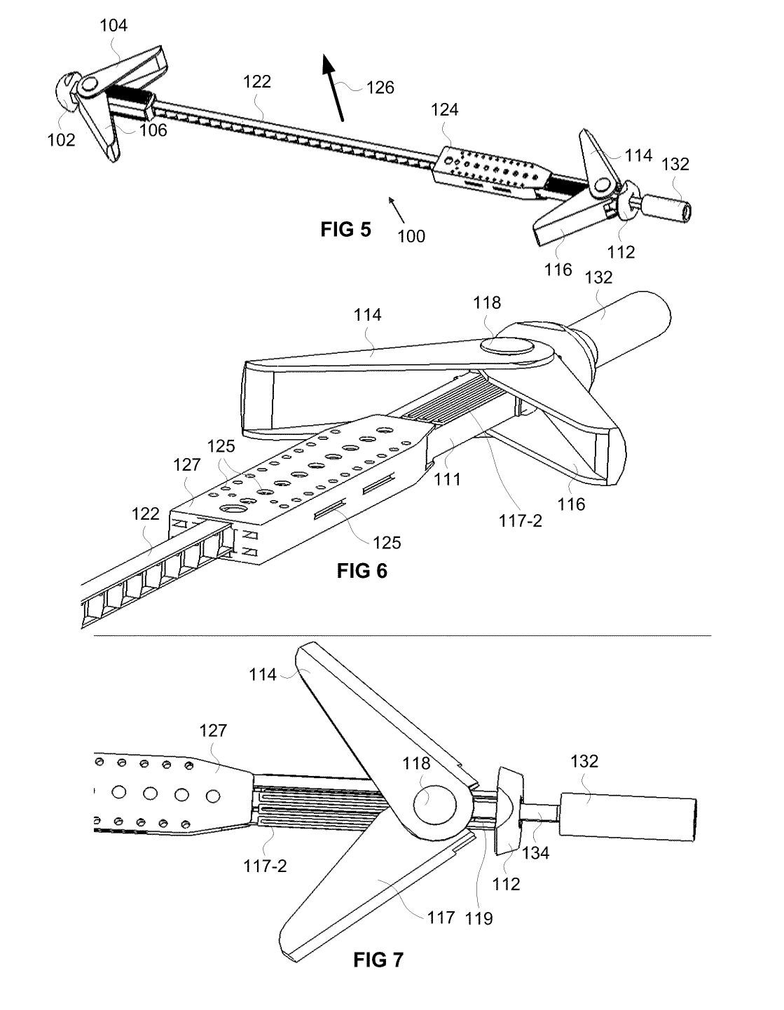

FIG. 5 is an overview of a device or instrument 100 according to a first group of embodiments of the invention. Variations may have different lengths (e.g., by varying the length of the toothed rail, etc.) in order to accommodate different surgical situations. The device depicted here is approximately 18 mm long. At the proximal end of the instrument may take the form of a wire connector 132 that is attached to a wire or cable that may be used to shorten the length of the device during a tissue approximation procedure. The device may include a proximal tip 112, which is preferably tapered to facilitate loading the device into a needle for delivery. At the proximal end are located a pair of wings, one narrow 116 and one wide 114. In some alternative embodiments, the wings may have a common width though this may have an impact on overall compactness of the device during its closed state. Both wings pivot, allowing transition from a open position (e.g., in which the wings may span approximately 4 mm) to a closed position (e.g., allowing the device to fit within a needle with a 1-mm inside diameter) and various positions in between. The narrow wing 116 may be designed to fit within the wide wing 114 to allow the wings to be as large as possible once opened, but as small as possible once closed. Springs 117-1 and 117-2 (e.g. see FIGS. 6 and 44) are provided to help spread the wings. A catch housing 124 may be provided which encloses catches which engage the teeth of a toothed rail. At the distal end of the device are located a second pair of wings 104 and 106 which may be similar to those at the proximal end. A tip 102 is provided at the distal end of device 100; this may be rounded to minimize tissue damage, turbulent blood flow around the device, and so forth, as well as loading into the delivery needle (if it is desired to load this end first).

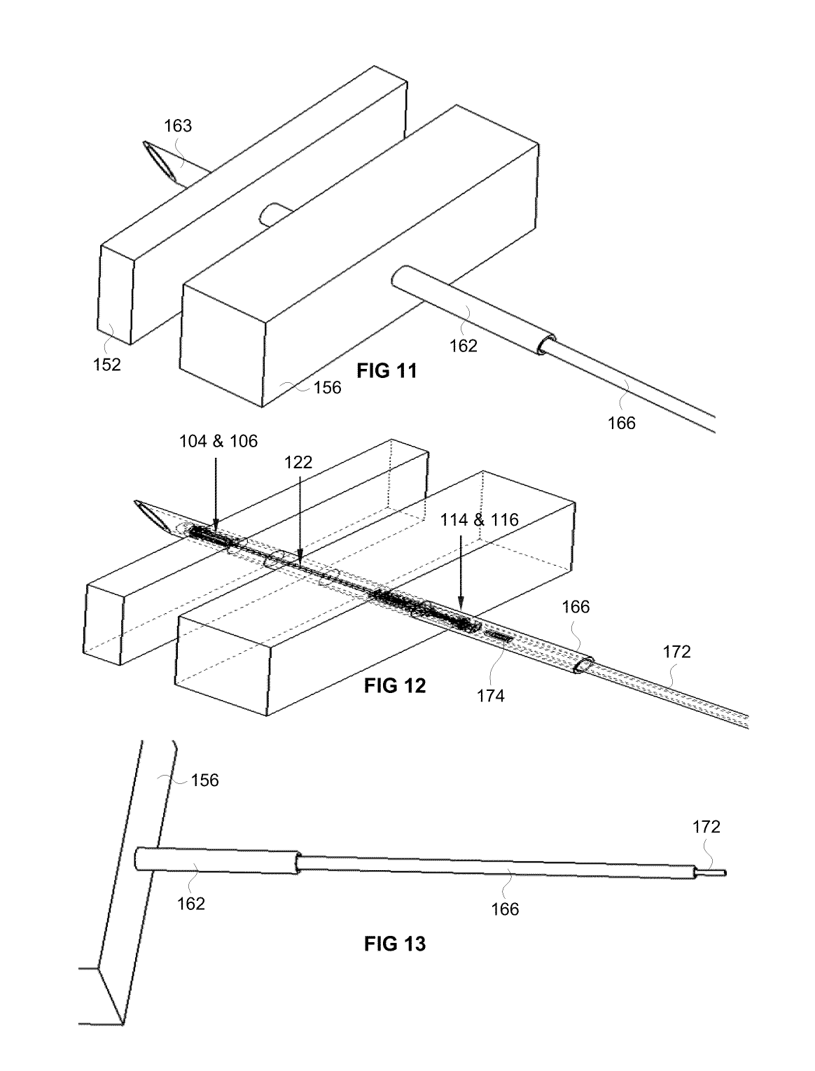

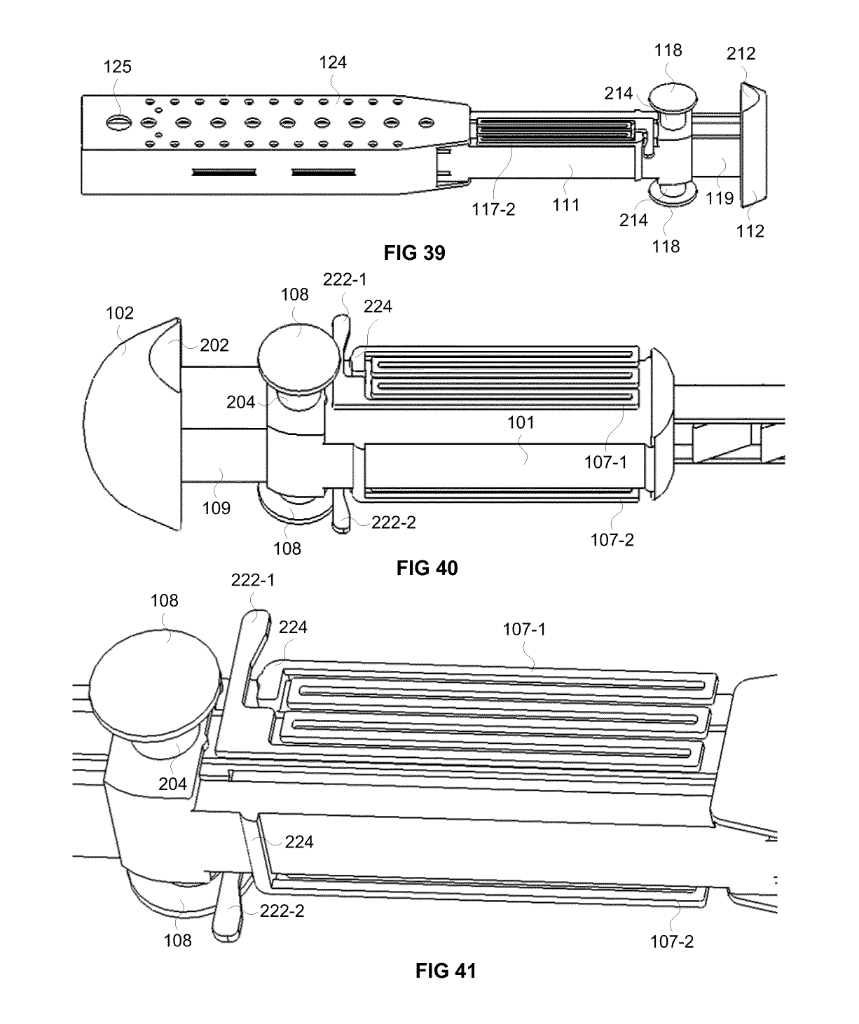

FIGS. 6 and 7 provide perspective and side views of the proximal end of the device. The two wings 114 and 116 are shown in partially open position; the wings may be fabricated in this position such that the springs are not pre-loaded until the device is inserted into the delivery needle. The position shown is also one that the wings may assume after the needle has been withdrawn such that the springs 117-1 and 117-2 have spread the wings. The two wings may share the same pair of pivots 204 having pivot caps 118 as shown in FIG. 39. In other embodiments separate pivots may be used. The proximal block 111 supports the pivots for the proximal wings 114 and 116 as well as the proximal springs 117-1 and 117-2 used to help spread these wings. The proximal block features a longitudinal channel to accommodate the toothed rail 122 and rail puller 134. At the distal end of the catch housing 127, the toothed rail 122 enters this channel. The catch housing 127 is continuous with the proximal block 111. Release holes 125 may be provided within the catch housing to facilitate complete release of sacrificial material if the device is fabricated using an electrochemical fabrication technology such as one of those discussed herein above or incorporated herein by reference (e.g. EFAB™ technology which is a layer-by-layer manufacturing process commercialized by Microfabrica Inc. (Van Nuys, Calif.) in which both structural and sacrificial material are deposited on each layer). Monolithic fabrication—without the need for assembly—using an electrochemical fabrication technology is assumed here, and the particular design under discussion takes into account the current design rules, process considerations, and capabilities of EFAB technology as implemented by Microfabrica. If desired, the delivery system (e.g., needle 162, push tube 166, and/or pull wire 172 (as can be seen, e.g. in FIGS. 11 and 16) can be co-fabricated along with the device.

However, other fabrication methods may be employed. Whatever method of fabrication is employed, unless the device is intended for relatively short-term use in the body, that portion of the device 100 which is to remain in the body should be made from a biocompatible material (e.g., nickel-titanium, titanium, stainless steel, tantalum, cobalt-chromium, or biocompatible polymer) or else coated with a biocompatible material. Methods for forming devices from such materials is described in U.S. patent application Ser. No. 11/478,934, filed Jun. 29, 2006, by Cohen et al., and entitled “Electrochemical fabrication processes incorporating non-platable metals and/or metals that are difficult to plate on”. This referenced application is incorporated herein by reference as if set forth in full herein. Assuming an electrochemical fabrication technology is used, the preferred axis 126 along which layers are stacked to fabricate the device is shown in FIG. 5. Of course stacking of layers along other axes is possible. A proximal extension 119 is shown in FIG. 7; this always passes through the apertures in the proximal wings regardless of their position, thus ensuring that the toothed rail and rail puller (which pass through the proximal extension) are able to pass through these apertures.

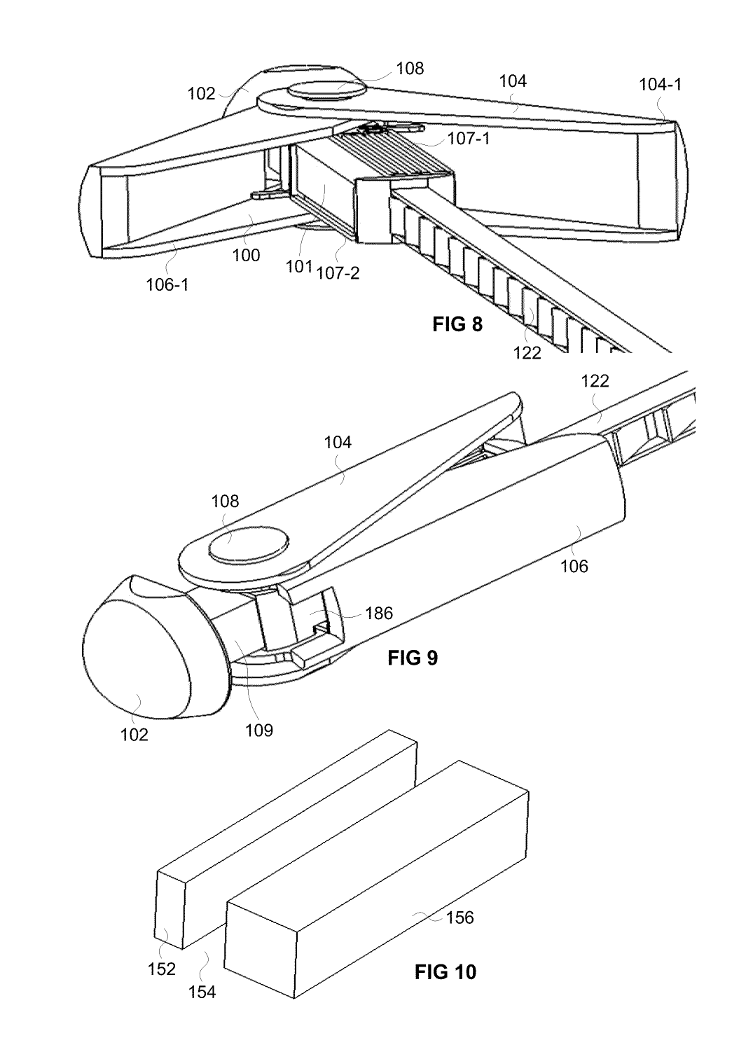

FIG. 8 provides a view of the distal end of the device. Again, the two wings 104 and 106 are shown partially open. One of the two springs 107-1 used to help spread the wings (as shown, the wide wing) is visible. The toothed rail 122 is connected to the distal block, which supports the pivots for the distal wings 104 and 106 and distal springs 107-1 and 107-2.

FIG. 9 provides another view of the distal end of the device. The wings are shown in their fully-closed position, which allows insertion of the device into a delivery needle 162 such as that shown in FIGS. 11-21. Visible is the aperture 186 in the narrow wing 106 through which the distal extension 109 passes, regardless of the position of the distal wings.

FIGS. 10-14 depict an initial sequence of operations illustrating the use of the device for approximating and holding together two walls of tissue, and also provide some addition views and details of the device. In FIG. 10 two walls of tissue 152 and 154 are seen, one proximal (i.e. 156) and one distal (i.e. 154). These may represent, respectively, the septum secundum and septum primum of the heart, which if separated after birth comprise a PFO. Approximating and holding together these septa will close a PFO and provide a cure to PFO-related illness.

In FIG. 11, a delivery needle 162 containing the device 100 has perforated both tissue walls 152 and 154. The tip of the needle is inserted far enough to ensure that the tips of the distal wings 104 and 106 will clear the distal surface of the distal wall 152, allowing the wings to spread. Also shown in the figure is a push tube 166 which fits within the needle; one function of this is to prevent retrograde (i.e., proximal) motion of the device as it distal and proximal ends are being brought together (i.e. as it is being shortened). With respect to PFO closure, the septum primum is typically 4-5 mm thick in adults (though as thick as 8-10 mm in some patients) and the septum secundum is typically 1-2 mm thick in adults, but can be 3-4 mm thick. The width of the tunnel defect between the septa is typically 3-5 mm, but can be as large as 10 mm, especially when stretched.

FIG. 12 provides a partially transparent view of FIG. 11, with hidden lines visible (i.e. the edges of the elements that were obscured from view) showing some components of the device within the needle.

FIG. 13 shows some elements of the delivery system in the region of the proximal end of device 100 prior to delivery of the device but after insertion of the needle into the tissue to be approximated. In this drawing the needle 162, the push tube 166, and the pull wire 172 are visible. These components are manipulated relative to one another to deliver device 100. While the proximal ends (e.g. the ends to be manipulated by a surgeon) are illustrated as being close to the proximal wall 156 for simplicity, in fact they may be located far (e.g., 1 meter) from the proximal wall, to allow delivery of the device at a significant distance from the entry port in the patient's body through which the device is introduced. The needle 162 may be made as long as desired, or for greater flexibility, may be relatively short and attached at its proximal end to a flexible tube (not shown) such as a catheter to enable use at a significant distance. Similarly, the push tube 166 (which may be, for example, 21 or 22 gauge in order to fit within the needle) may also itself be long or else attached to a flexible tube. The wire 172 would ordinarily be flexible enough that no flexible extension is required. Preferably before delivery of device 100, the relative lengths of the needle (or its attached tube), the push tube 166 (or its attached tube), and the wire 172 are such that all three components are exposed and accessible over a sufficient distance from their proximal ends, allowing, for example, one component to be moved while another is held.

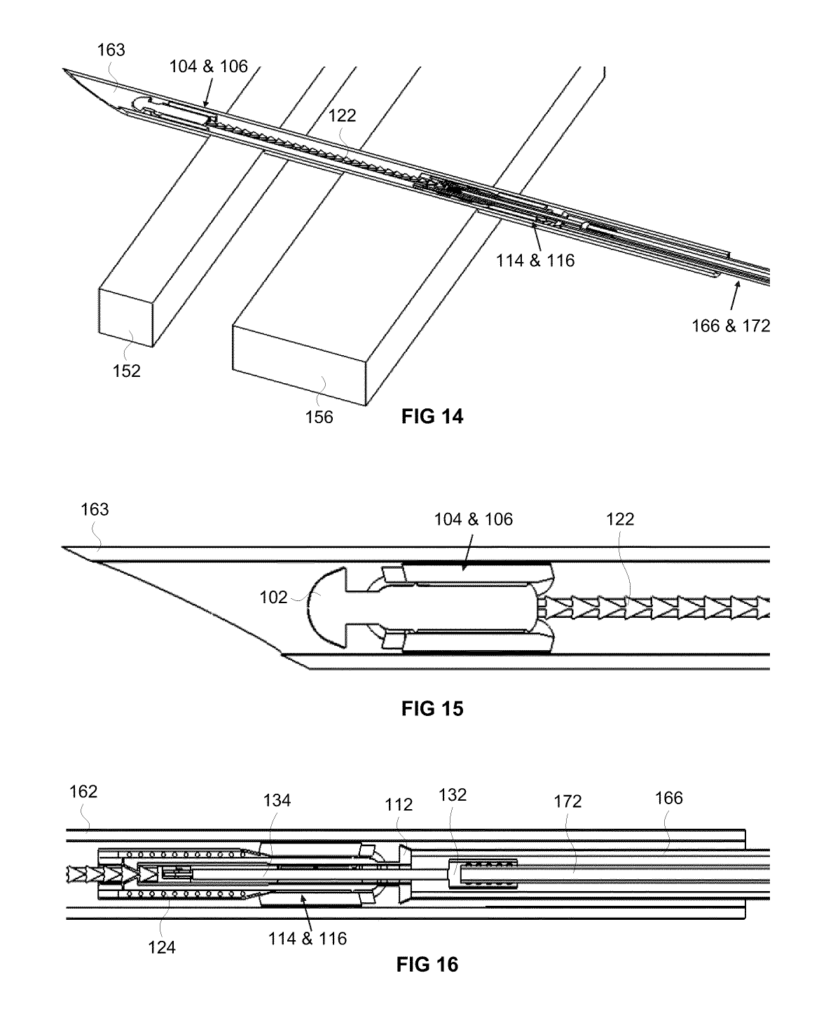

FIG. 14 provides a sectional view of the elements of FIG. 11, showing the device 100, along with the push tube 166 and pull wire 172, within the needle 162. The proximal wings 114 and 116 and distal wings 104 and 106 are shown in closed position. In FIG. 14 the tip 163 of needle 162 may also be seen along with toothed rail 122.

FIG. 15 provides a sectional view of the distal end of the device within the needle, whereas FIG. 16 is a sectional view of the proximal end of the device within the needle. The rail puller 134 is shown interfaced to the toothed rail 122 at the distal end; the proximal end of the puller 134 is continuous with the wire connector 132. The pull wire 172 is attached to the wire connector 132 by means known to the art such as adhesive, solder, or other bonding material; alternatively the wire may be welded (e.g., laser welded using a small spot size beam), brazed, or crimped to the connector, or the connector may include mechanical features which capture the end of the wire (e.g., if flared or bent). The connector 132 may be provided with features (not shown) which facilitate attachment to the wire, such as side apertures which allow access by a focused laser spot, the application of solder, or other bonding material to both connector and wire, etc. As may be seen, the distal end of the push tube 166 is able to come into contact with the proximal tip 112 of the device; this prevents excessive retrograde (i.e., proximal) motion of the device when it is being shortened during delivery.

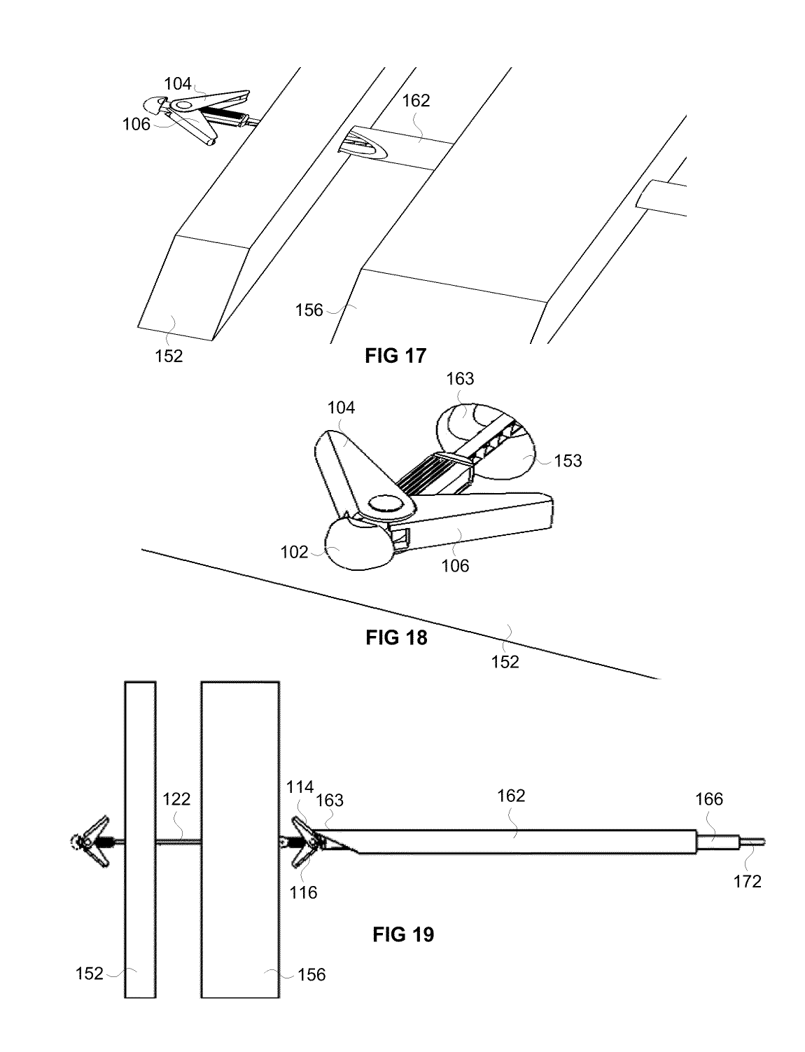

In FIGS. 17-18, the delivery process has continued with the needle partially withdrawn, while the push tube is held to prevent retrograde motion of the device or implement. Once the distal wings have cleared the needle tip, the distal wing springs 107-1 and 107-2 spread the wings to at least a partly-open position. It should be noted that the size of the perforation in the wall left behind by the needle will not necessarily be as large as that shown in FIG. 18 and in the other figures; the tissue may recoil such that the size diminishes once the needle is withdrawn.

In FIGS. 19-20, the delivery process has progressed further such that the needle 162 has been withdrawn enough for the proximal wings 114 and 116 to clear the needle tip 163 and springs 117-1 and 117-2 to cause wings 114 and 116 to partially open.

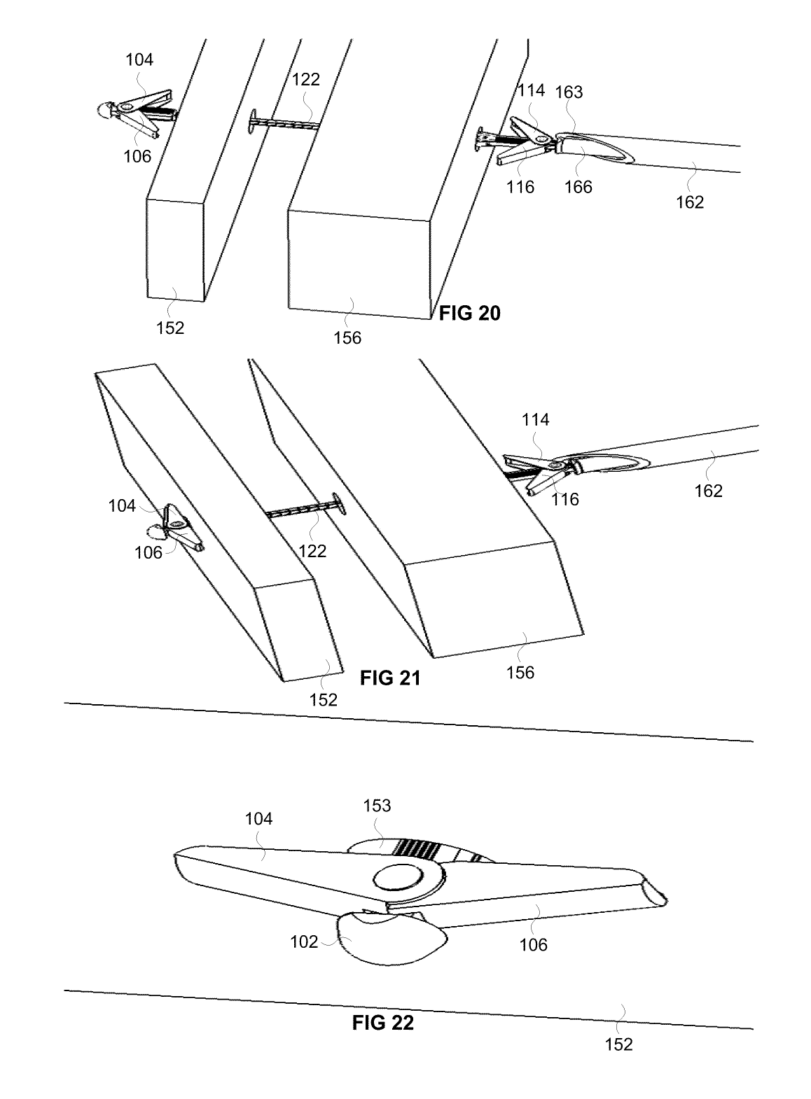

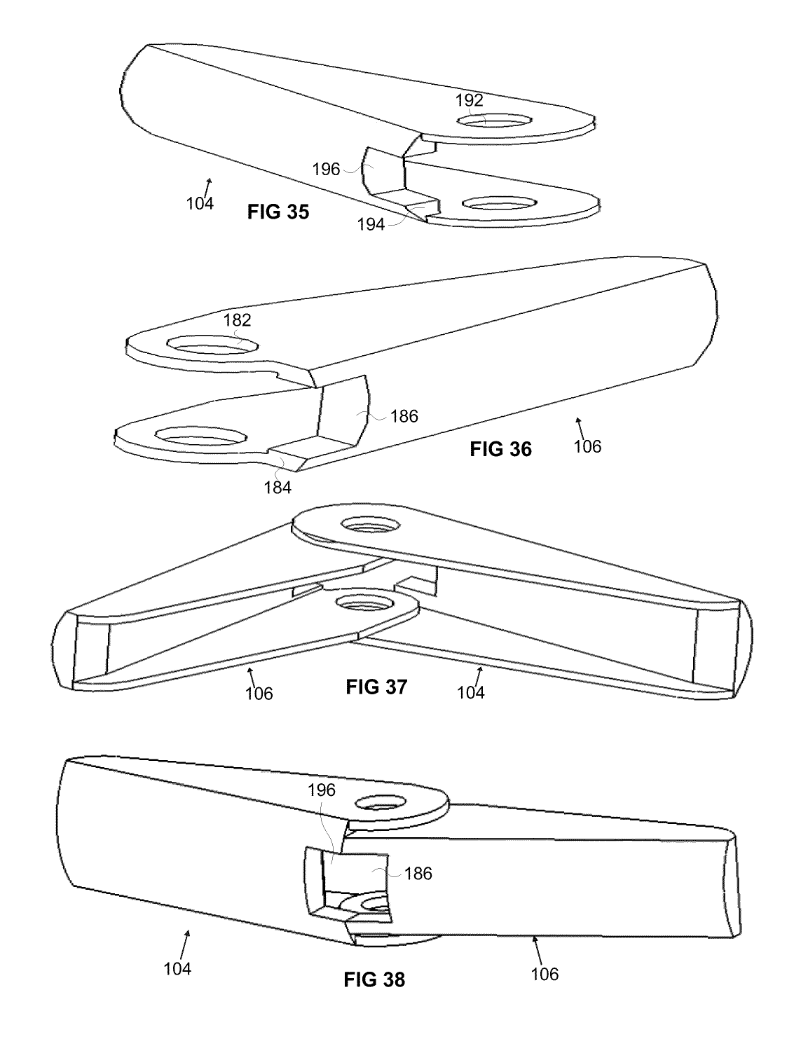

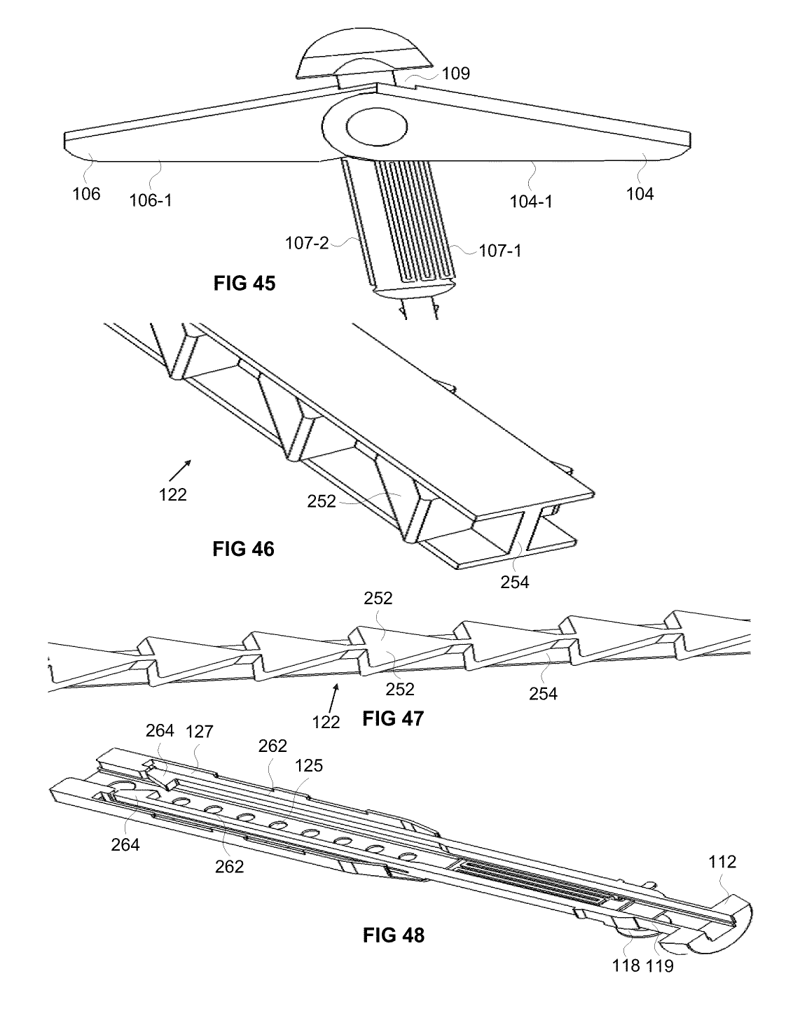

In FIGS. 21-22, the pull wire 172 has been pulled such that the distal end 102 of device 100 has been drawn toward the distal side of distal wall 152. Contact between the wings 104 and 106 and the wall 152 completes the process of opening the wings, such that the tissue contact surfaces 106-1 and 104-1 (e.g. see FIG. 45) of the wings are at least partly in contact with the wall 152. In this configuration, the tissue contact surfaces 106-1 and 104-1 of the wings 106 and 104 may be at a large angle (e.g., 180 degrees) with respect to one another. The mating surfaces 194 and 184 (e.g. see FIGS. 35 and 36) of the wide and narrow wings 104 and 106 may also be in contact in this configuration. The tips of the wings are preferentially curved such that contact with the wall 152 is not traumatic to the wall tissue. In some other embodiments, it may be desirable to have the tips embed themselves in the wall and thus tips with a greater biting configuration may be used. In the present embodiment, the tip configuration encourages the wing tips to slide over the wall surface and cause the wings to open fully. Since extended wings span a significant distance (e.g., approximately 4 mm in the design depicted in the figures) compared with the width of the perforation left in the tissue wall by the delivery needle, the wings cannot be pulled beyond the wall surface that they engage (other than by damaging the tissue and/or the device). Thus the expanded wings provide an anchoring function for the device on the surface of the tissue. When partially open, the distal wings may also be spread, if desired, by moving the device relative to the distal tip of the needle such that the needle tip pushes the wings open.

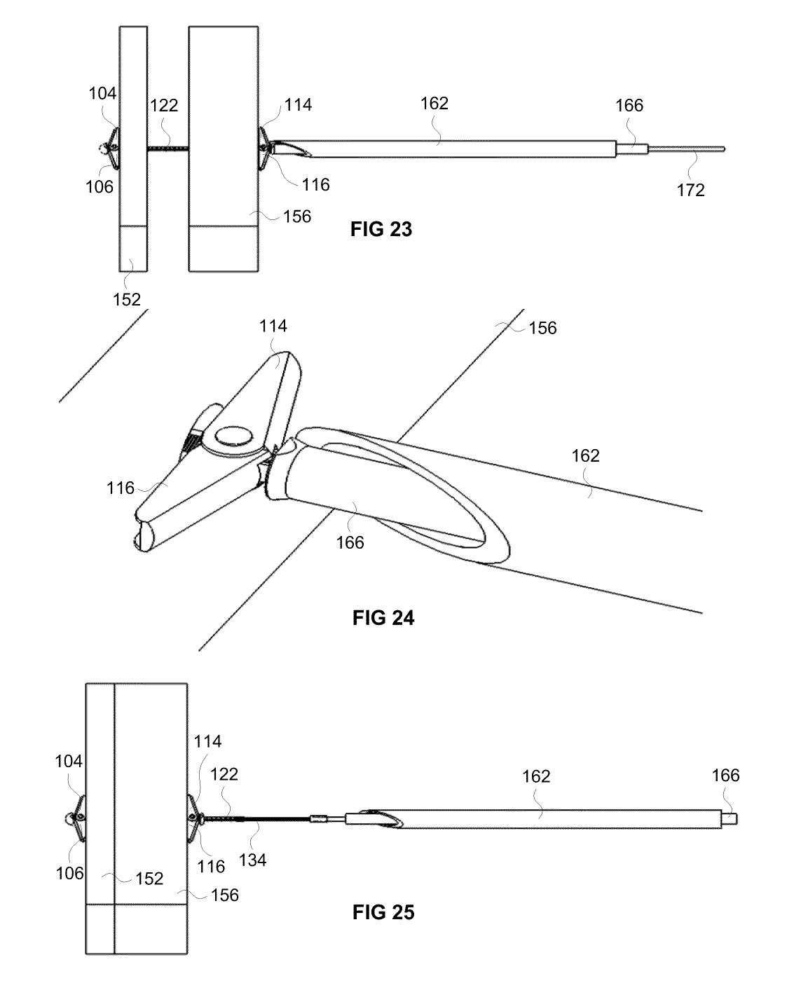

In FIGS. 23-24, the delivery process has progressed still further; the pull wire has been pulled further, and/or the push tube has been advanced, such that contact between the tips of the proximal wings and the wall has occurred and the wings have been completely opened, with their tissue contact surfaces at least partly in contact with the wall.

In FIG. 25, the pull wire has been pulled further such that the tissue walls are pulled together, eliminating or reducing the separation between them.

In alternative embodiments, the process set forth above for approximating tissue elements may be performed in different ways. For example, the proximal wings may be pushed toward the proximal wall by advancing the push tube before the distal wings have contacted. Rather than pull the pull wire, the pull wire may be held in place with respect to some reference (e.g., the patient) and the push tube may be pushed, forcing the proximal wings to engage the proximal wall and (at least once the gap between the walls has been closed) forcing the distal wall to engage the distal wings. Or, both the proximal and distal wings may contact the tissue and be spread open at approximately the same time. Or, the distance between the walls may be reduced by pulling on the wire before the proximal wings have fully engaged the proximal wall. Whatever approach is used, the result is that there is relative motion between the toothed rail and the catch housing causing the device to become shorter, the wings to extend, and the separation between the walls to be eliminated or reduced.

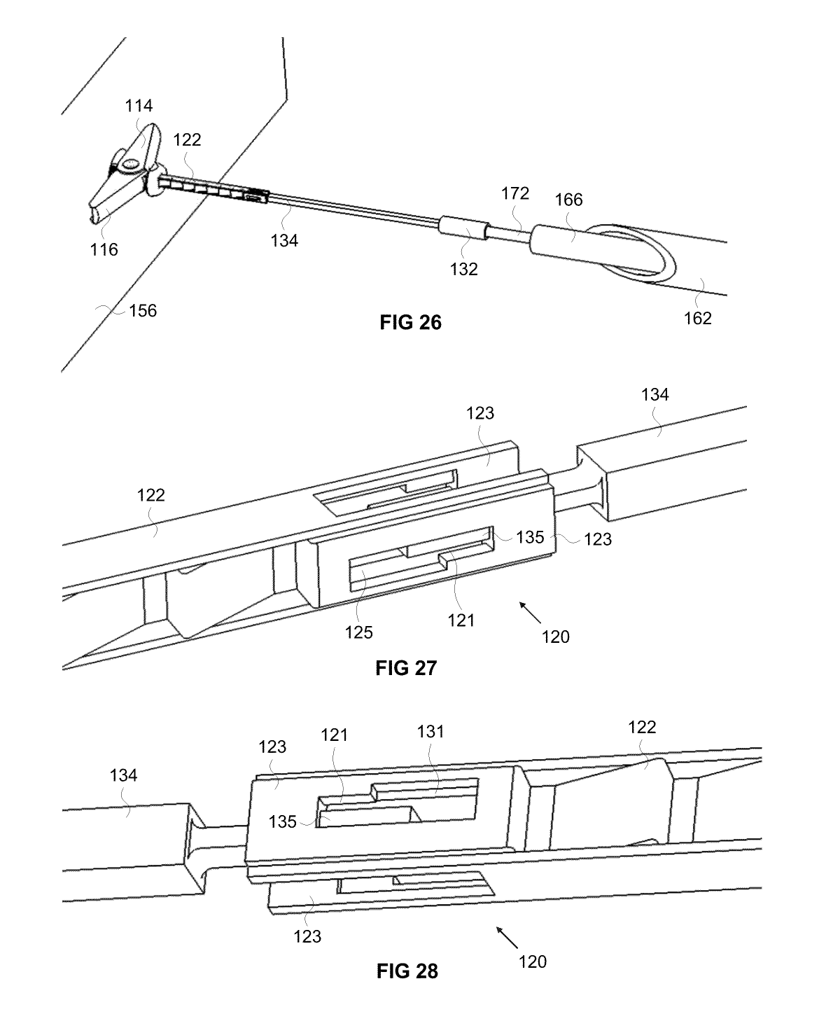

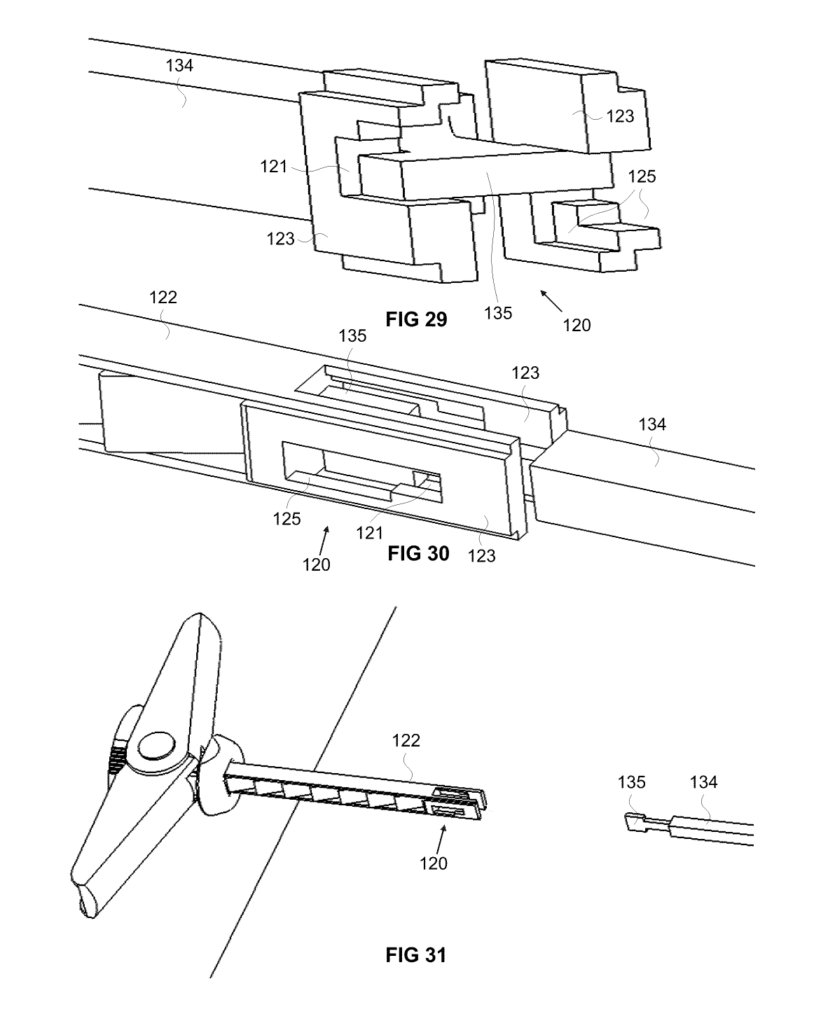

In FIGS. 25-26 the needle and push tube have been withdrawn further for purposes of illustrating the interface between the rail 122 and rail puller 134 and how one may be separated from the other after delivery of the device. With the design of the interface described here, no further withdrawal of the needle or push tube is actually required to effect this separation, though other designs may utilize withdrawal of one or both components.

The interface between the rail 122 and rail puller 134 is seen in detail in FIGS. 27-28. Two parallel prongs 123 are provided at the proximal end of the rail 122. The rail puller 134 is terminated at its distal end with a rectangular lug 135. Each prong 123 includes a lug slot 121 designed to accommodate the lug 135 when it is engaged, as well as lug clearances 131 (cutouts in the wall) which allow rotation of the lug by approximately 90 degrees from an engaged position (fully clockwise as seen from the rail puller) to a disengaged position (fully counterclockwise). The lug slots and clearances in one prong are rotationally symmetric with respect to those of the other prong, with the axis of rotation coincident with the longitudinal center axis of the toothed rail.

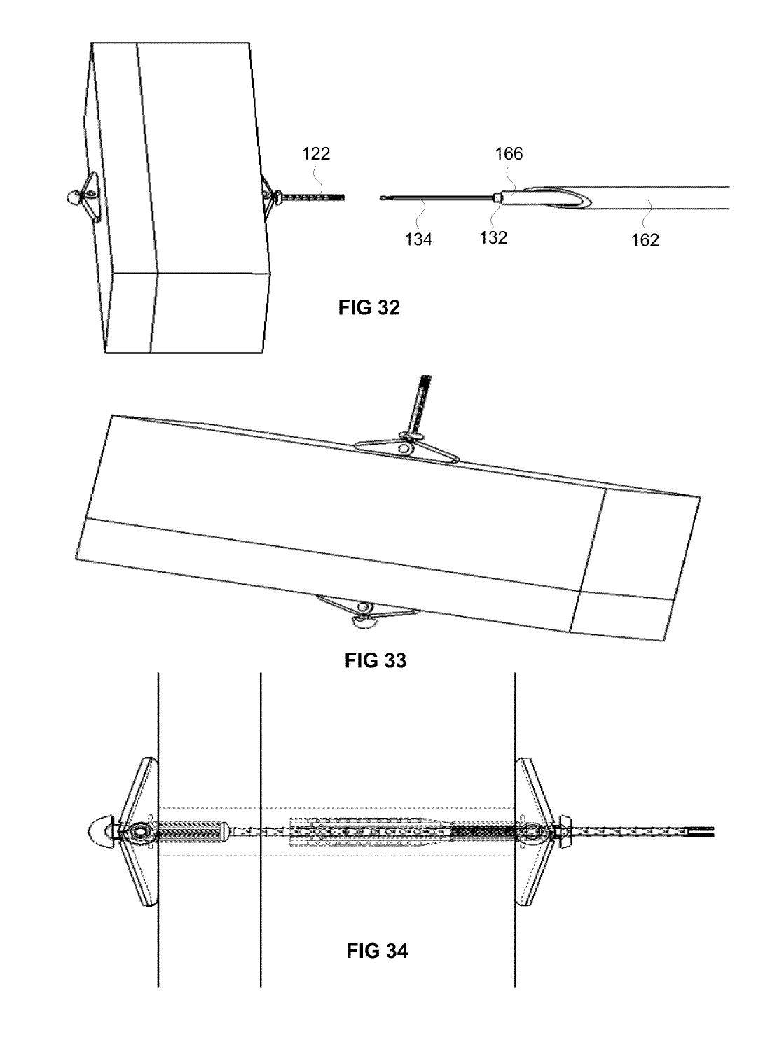

To couple the rail puller 134 to the rail 122 (i.e., to engage the lug), the puller is pushed sufficiently distally that the lug 135 is free to turn within the lug clearances 131, rotated 90 degrees clockwise (as seen from it) and pulled proximally a short distance so that the lug 135 enters the lug slot, within which it is unable to turn. To decouple the rail puller from the rail after the device is delivered, as shown in FIGS. 31-32, the puller is pushed distally a short distance, then rotated 90 degrees counterclockwise, then pulled out completely (at this time the lug is approximately parallel with the prongs). If the device is fabricated using EFAB technology and the rail puller is fabricated as an integral part of it, then it may be fabricated in the disengaged position (assuming the design shown) or in an engaged position (assuming a modified design). The shaft of the rail puller is small enough in cross-section to rotate within the proximal block and catch housing when the proximal end of the rail is still within these structures (i.e., if the device is only shortened by a small amount).

FIGS. 33-34 illustrate the device 100 and the tissue walls 152 and 154 after it has been delivered and decoupled from the delivery system. FIG. 34 provides a perspective view showing hidden lines.

In practice the toothed rail 122 may or may not extend a significant distance from the proximal tissue wall or a significant distance beyond the proximal tip. In some embodiments, the length of the rail may be dictated by a desire to have the rail and a catch head 264 (see FIG. 48) engaged during the entire deployment of the device. In other words, in such embodiments, the length of the rail would be selected so that insertion of the distal end through the tissue would be far enough to allow the wings to open while having the distal and proximal tissue walls located in their non-approximate positions while engagement exists In other embodiments, it may not be necessary for the toothed rail to engage the catch head of the proximal end of the device while the insertion occurs and even while spreading of the wings occurs or even during partial approximation occurs. In some of these embodiments, engagement of the rail with the catch head need only occur before approximation is completed. In such cases the rail may need not extend from the proximal end at all or only slightly (i.e. enough to ensure engagement given tolerances in tissue thickness and the like.

In practice, multiple devices may be delivered to a site (e.g., a PFO), and implanted in an appropriate pattern to approximate and retain a larger region of tissue than a single device could do on its own. Such devices may be delivered by extracting the delivery system and reloading a device into it after each delivery or by having a delivery system that can hold and sequentially deploy multiple devices.

FIGS. 35-36 provide perspective view of the wide and narrow wings, respectively. Holes for the pivots which allow wing rotation are provided. Each wing has a mating surface 194 (wide wing) and 184 (narrow wing) which mates with the mating surface of the other wing when two wings on the same pivots are fully opened. Each wing also has an aperture 196 (wide wing) and 186 (narrow wing) which allows the proximal or distal extension to pass through.

FIGS. 37-38 show the wide and narrow wings (either proximal or distal) assembled together as they are in the actual device, with openings aligned to share pivots. In FIG. 37, the wings are partially open, while in FIG. 38, they are fully open, with their mating surfaces in contact.

Each pair of wings is assembled onto pivots at either the proximal end (as can be seen in FIG. 39) or the distal end (as can be seen in FIG. 40) of the device. If formed according to some of the embodiments described herein, the wings may be fabricate with their pivot openings in place around pivot 204 or 214. All pivots 204 and 214 are provided with caps 108 and 118, respectively, to prevent the wings from escaping from the pivots. In other embodiments, however the cap may take on different shapes or be removed in its entirety. The proximal and distal tips 112 and 102 may be provided with flats 212 and 202 as shown to minimize the total fabricated height of the device (e.g., the number of layers), thus reducing cost when using a multilayer fabrication method. Both the proximal and distal blocks 111 and 101 support the pivots and are each provided with a pair of planar meandering extension springs 117-1 and 117-2 and 107-1 and 107-2, respectively. The spring (e.g. 117-1 or 107-1) on one side of the block is rotationally symmetric with respect to the spring (117-2 or 107-2) on the opposite side of the block, around a longitudinal axis passing through the center of the block.

FIG. 41 provides an even more expanded view of the distal wing pivots and spring elements.

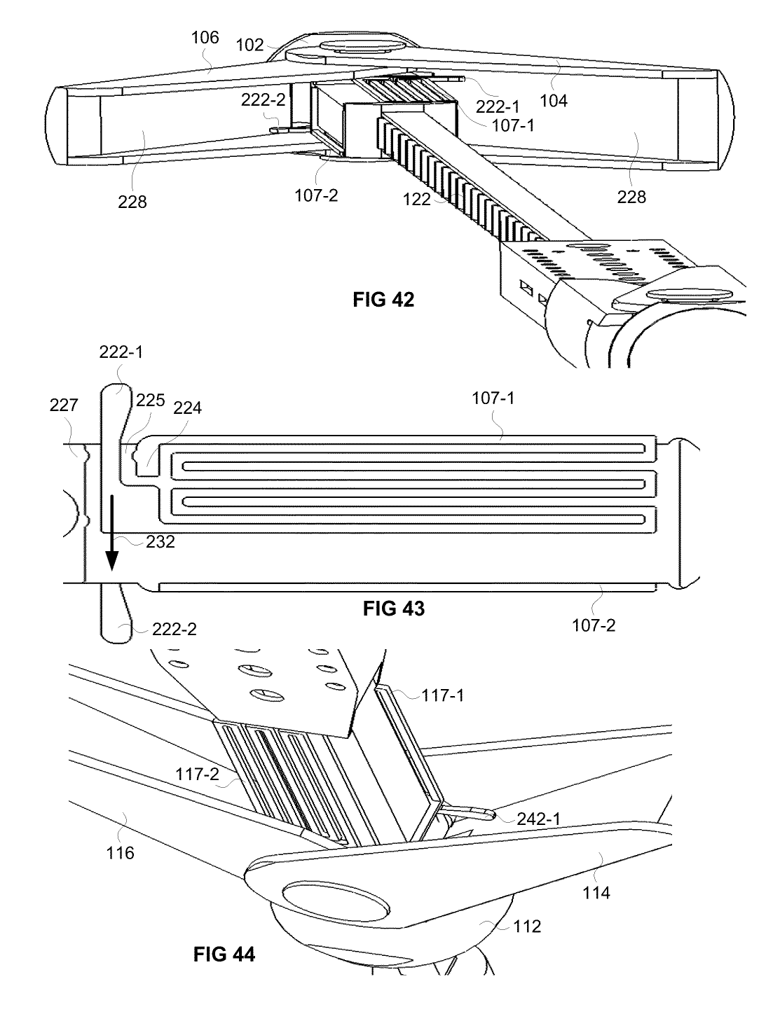

FIG. 42 provides another perspective view of the distal portion of the device such that the engagement between spring tips and wings can be seen.

FIG. 43 provides an even more expanded view of one of the distal elements.

FIG. 44 provides another perspective view of the proximal portion of the device such that the engagement between a spring tip and a wings can be seen.

As can be seen in FIGS. 40-44, each spring includes a spring tip 222-1. 222-2, or 242-1 (the fourth spring element is not visible) which is intended to engage the inner surfaces 228 of the distal wings or the inner surface (not labeled) of proximal wings. The spring tips are rounded to encourage sliding against the inner surfaces as the wings close and open. In the sectional view of FIG. 43, guides 227 may be seen to help guide the travel of the spring tip when the spring extends and relaxes. The ideal direction of travel 332 of the spring tip as the spring extends (due to the associated wing moving toward a closed position) is also shown; the actual travel of the tip may be somewhat different, and the orientation of the tip may change as it moves. To load the device into the delivery needle, the wings are moved to the closed position, causing movement of the spring tip and extension of the springs, thus pre-loading the springs. When the needle is later withdrawn as discussed above, the extended springs are able to relax, pushing the wings with their tips toward a partly open position or a fully open position (e.g. if the wing is ‘launched’ by the force of the relaxing spring). When the device is not inserted in the needle and if no other force acts to close the wings, the wings may be in a position such that their inner surfaces rest against the tips of the relaxed springs (FIG. 44). The base of the springs is fixed to the proximal and distal blocks as shown in the figures. In other embodiments, other spring designs may be used including designs that attach spring elements to the wings as opposed to the blocks.

As shown in FIG. 45 (a view normal to the pivot cap top surface), when the wings are fully open the apertures within them can be designed large enough such that the extended wings can rotate as a unit with respect to the longitudinal axis of the device, allowing the tissue contact surfaces to make contact with the tissue in cases they might not otherwise do so. Providing for rotation of the wings may be important since the device may not be delivered perfectly normal to the surface of the tissue walls, and indeed, the tissue walls may not be parallel to each other. In the design illustrated here, rotation of approximately +/−10 degrees is provided for, and larger angles are possible with modified designs.

FIG. 46 shows a sectional close-up of the toothed rail. It can be seen that the rail may have a cross-sectional shape 254 similar to an I-beam if stiffness against bending in both axes is desired (e.g., to prevent permanent, plastic distortion of the rail during handling, which might prevent the device from shortening during delivery). In other embodiments, flexibility in at least one axis may be desirable. Teeth 252 may be provided symmetrically about the centerline of the rail, partially recessed within the crossbars of the “I” as shown.

FIG. 47 provides a sectional, perspective view of the rail with one of the crossbars removed, providing a better view of the teeth 252. The teeth 252 may be designed at a pitch suitable to provide the minimum increment of adjustment in device length after shortening. In other embodiments, the teeth may not be symmetric but instead, for example, they may exist on only one side of the rail while the other side is smooth.

FIG. 48 provides a sectional perspective view of the proximal end of the device with wings removed, showing the proximal block and catch housing 127. Inside the catch housing are two catches designed to engage the teeth of the toothed rail and allow movement of the rail relative to the proximal block in the proximal direction only, in a ratcheting fashion. The catches comprise catch beams 262 terminated distally with catch heads 264 and proximally anchored at their bases to the proximal block.

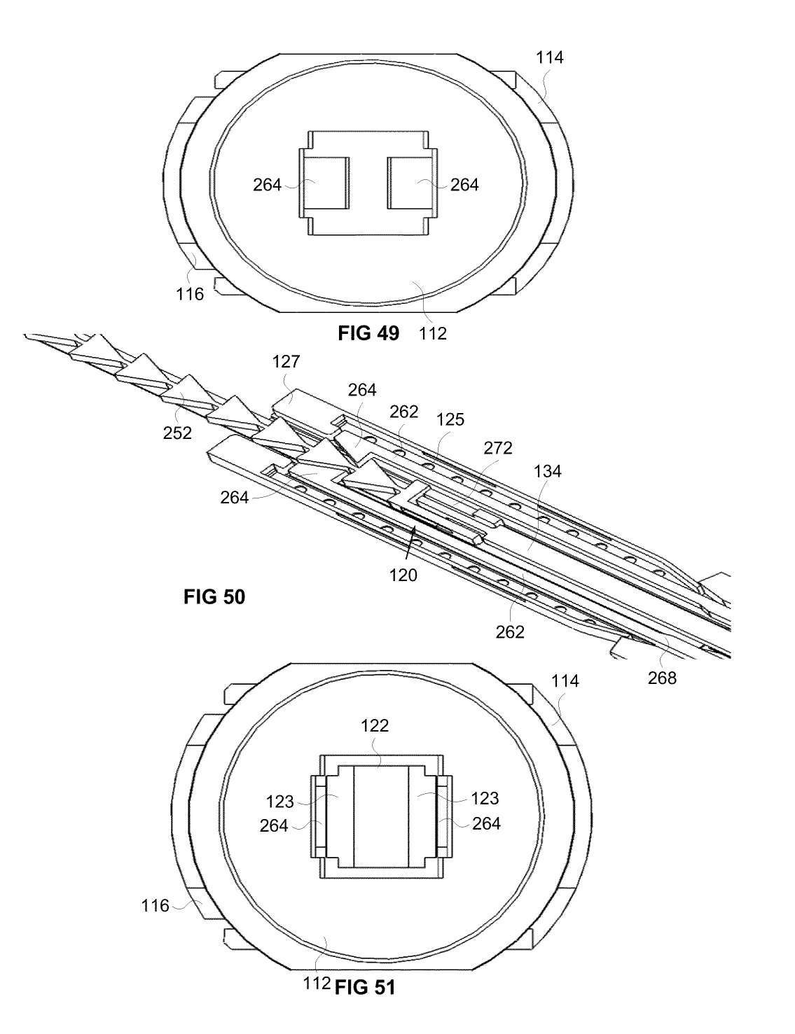

FIG. 49 provides an end-on view of the proximal end of the device (with wings in the closed position), showing the channel through which the toothed rail passes, as well as the heads of the catches which extend into the channel to engage the teeth.

FIG. 50 is similar to FIG. 48, but with the rail 122 and rail puller 134 added. As may be seen, the catch heads 264 are arranged so as to engage the teeth of the rail. When the rail is moved distally with respect to the proximal block (e.g., by pulling on the rail puller 134 with the pull wire 172), the catch beams deflect away from the device centerline along their entire length beginning just distal to their bases, to allow rail motion that shortens the device. However, when tissue pressure against the wings attempts to move the rail distally with respect to the proximal block, the nearest tooth is engaged by the catch heads and the rail is prevented from moving. The stiffness of the catch beams and the angle of the teeth and catch heads should preferably be designed such that an appropriate level of force is required to move the rail with respect to the proximal block and shorten the device. If this force is too high, device delivery may be compromised and the force required may become too large a fraction of the tensile strength of the device and/or delivery system. If the force is too low, however, then the device might inadvertently shorten during loading into the needle, if the pull wire snags when the push tube advances the toggle toward the delivery site, etc.

The rail may be monolithically-fabricated along with the other parts of the device using an electrochemical fabrication technique or similar method; in the position shown in the figures, the rail teeth have sufficient clearance with respect to the catch heads to allow for this.

FIG. 51 is similar to FIG. 49, but the rail has been added to the channel.

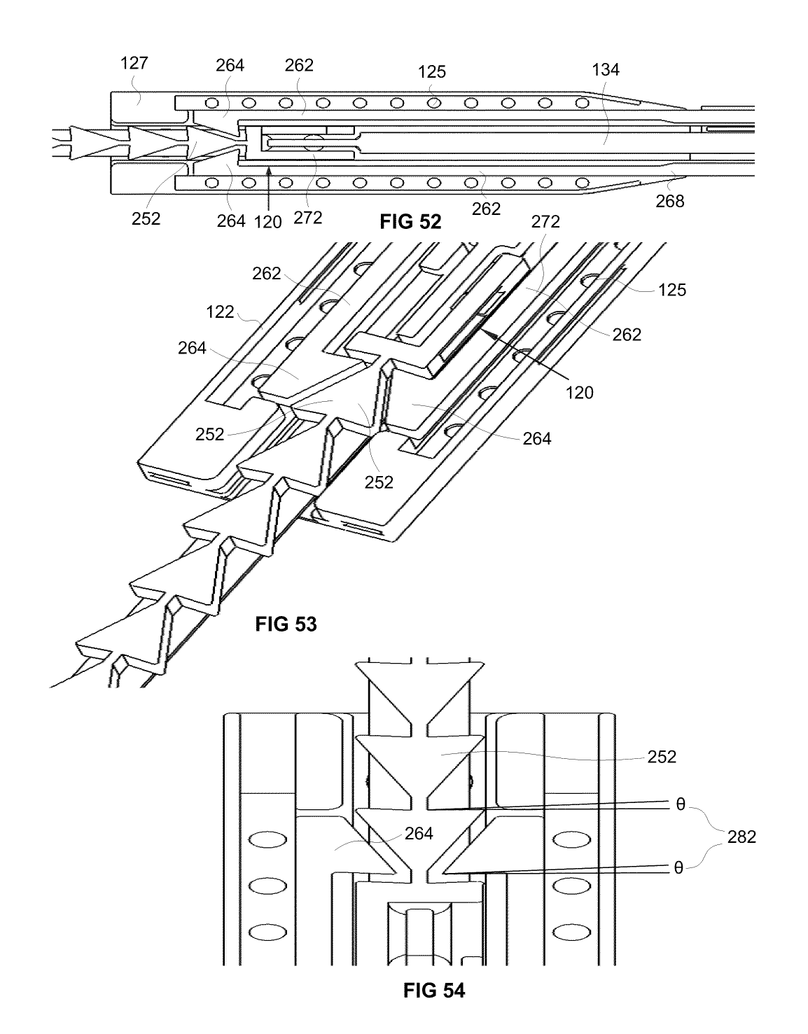

FIGS. 52-54 show other views of the toothed rail 122, catches, catch housing 127, and other elements of the device. The catch housing 127 serves in part to prevent possible impingement of tissue on the rail in the vicinity of the catch head 264, which may interfere with the catch heads adequately engaging the teeth. The housing also serves to keep tissue from impinging directly against the catch heads and rails, potentially impairing their motion.

FIG. 54 shows a sectional view of the rail teeth 252 and catch heads 264. The teeth and catch heads may be designed with a small re-entrant angle 282, labeled as θ (i.e. theta) with respect to the plane transverse to the rail; this angle may serve to generate a force on the catch heads that pushes them toward the device centerline when the device is subject to tensile loading. This force can help counteract any tendency for the catches to otherwise be deflected away from the centerline—potentially allowing the rail to move distally with respect to the proximal block—when the device is subject to large tensile forces.

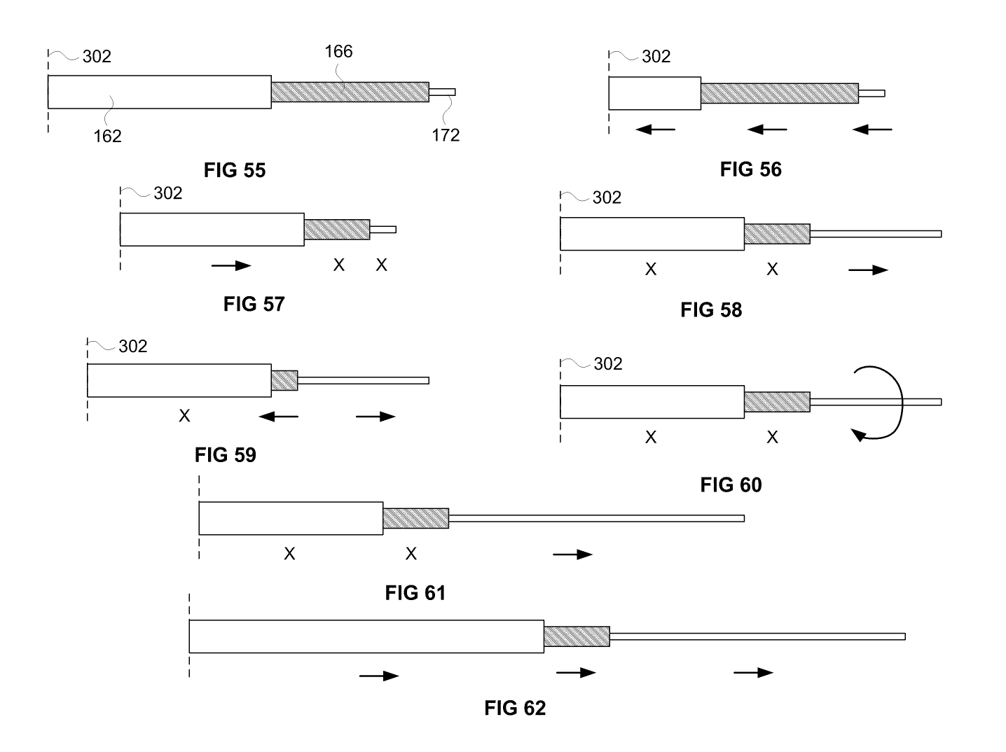

FIG. 55 shows the components of the delivery system, apparatus, or tool at their proximal ends, as well as a reference 302 (e.g., a port in the patient's body) with respect to which these components may be moved. This system includes a delivery needle 162, push tube 166, and a pull wire 172.

FIGS. 56-60 depict motions of these ends associated with the device delivery process. An arrow beneath a component indicates the direction in which the component has moved in order to arrive at the position shown in the figure, whereas an “X” beneath a component indicates that the component has not moved (in some cases the component has been actively maintained in the position shown).

In FIG. 56, the device and delivery system have been advanced toward the delivery site by advancing the needle, push tube, and pull wire, such that the needle penetrates the tissue walls as already described. The needle and push tube may be advanced by pushing on them on the tubes to which they may be attached. The wire need not necessarily be pushed, since the forward motion of the device caused by pushing on the push tube (and perhaps needle, due to friction) should ordinarily drag it along unless the force required to deflect the catch heads is too light, the wire snags, etc. In FIG. 57, the needle has been withdrawn to allow the wings to spread as described above. The needle may be fully withdrawn from the patient at this time if desired. In FIG. 58, the wire has been pulled to shorten the device; alternatively, in FIG. 59, the wire has been pulled, and the push tube has been pushed, so as to shorten the device, but with less retrograde (i.e., proximal) motion of the device and tissue. In FIG. 60, the wire has been twisted in preparation for releasing the rail puller from the toothed rail. In FIG. 61, the wire has been withdrawn, disconnecting the device from the delivery system. At this point the wire may be withdrawn fully from the body. In FIG. 62, the remaining components of the delivery system have been withdrawn. The step shown in FIG. 61 may be skipped, since the wire will be withdrawn anyway in the step shown in FIG. 62.

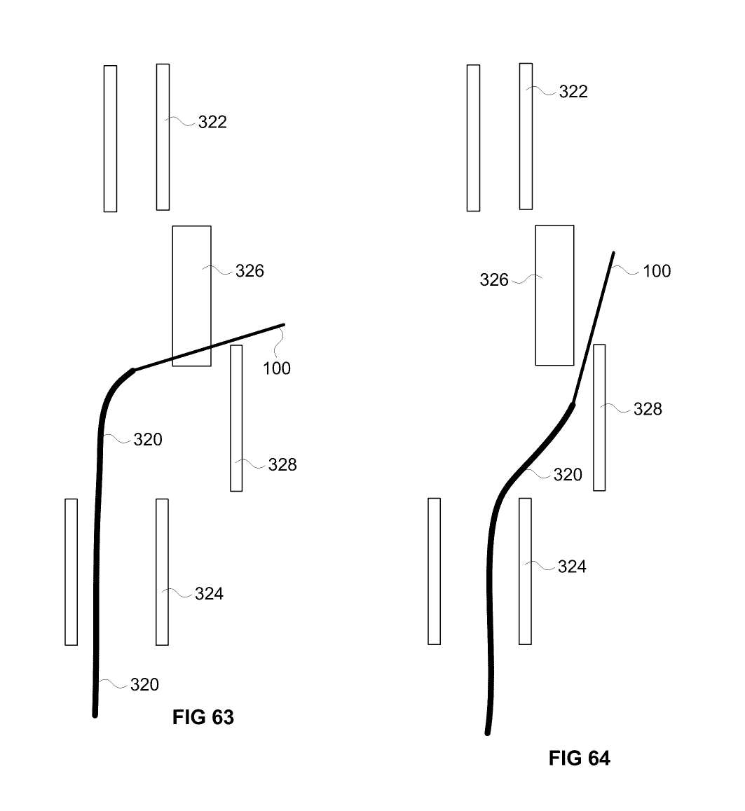

For PFO closure, a preferred approach to delivering the device would be percutaneous, e.g., guiding the delivery system 320 through a catheter into the heart. The PFO could be approached either through the superior vena cava (SVC) 322 or the inferior vena cava (IVC) 324, the latter being commonly used for PFO devices mentioned earlier. However, as shown in FIG. 63, approach through the IVC 324 may lead to penetration of the device 100 through the septum secundum 326 but not through the septum primum 328, especially when the overlap between septa is small or the separation between them large. Alternatively as shown in FIG. 64, an IVC approach may lead to the device sliding through the separation between septa instead of penetrating them both. By comparison as shown in FIG. 65, an approach via the SVC 322 may provide an improved angle to facilitate penetrating both septa as desired. A further benefit to approaching the PFO through the SVC is that the path length from the port is shorter. If the angle at which the device penetrates the tissue wall is large as shown in FIG. 65, the angle by which the wings can rotate about the longitudinal axis of the device may be inadequate to assure good apposition of the tissue contact surfaces with the wall if the spread wings lie in the plane of FIG. 65. However, since the distal and proximal wings lie in the same plane, the device can be rotated around its longitudinal axis (e.g., by twisting the pull wire, preferably clockwise to minimize the risk of disengaging the rail puller) until the spread wings are, for example, perpendicular to the plane of FIG. 65.

First Group of Embodiments—Further Alternatives:

Different embodiments are possible based on making various modifications to the design. For example, in the figures the proximal wide wings and distal wide wings are shown to be on the same side of the device; the proximal wide wing can be on one side of the device and the distal wide wing on the other side. It is not strictly necessary to have two wings at each end of the device; one wing may suffice to anchor the device, and may have some benefits. Alternatively, more than two wings may be advantageous, especially by allowing wings to be less than 180 degrees apart (with respect to the longitudinal device axis). The location of the catch heads and bases of the catch beams can be reversed in the sense that the heads are proximal and the bases are distal. One or more pivots whose rotation axis is parallel to the longitudinal axis of the device, or to some other axis, may be provided (e.g., between the toothed rail and the distal block) to allow rotation of the plane of one set of wings with respect to the other. Such rotation may be driven or be the result of the wings self-adjusting their orientation according to their local environment. The planar meandering springs shown in the figures may be replaced by other spring designs, including torsional springs of the sort that are commonly used in toggle bolts to spread the wings of these devices. The wings may also be spread to an open or partially-open position by mechanisms that employ the shortening of the device to actuate the wings, such as rack and pinion (see FIG. 74) and linkage mechanisms. If tissue recoil is sufficiently large such that the perforation size is considerably smaller than the distance between closed wing tips, or if a different wing shape is used, it is possible to eliminate the springs altogether, such that merely pulling the wings against the tissue wall serves to open them from a substantially closed position. Springs can also be eliminated if another method of opening the wings, such as inertial reaction of the wings to vibration, gravity, or other acceleration (perhaps in conjunction with a ratcheting mechanism that allows the wing to only open, but not close), or magnetism (applied through the patient's body from an outside source, or applied through the device) is employed.

Narrow and wide wings can be made to spread themselves into an open position through magnetic repulsion or magnetic attraction in lieu of a mechanical spring, depending on which side of the pivot the force is acting. For example, if the wings are magnetized so that both wings have their North pole facing one another with the force produced on the wing tip side of the pivot, then the wings will repel one another when in a closed position and when the device is released from the needle, the wings will spread open. Alternatively, magnetic attraction may be used to open and spread the wings. For example, the wing mating surface of the wide wing may be made a North pole and that of the narrow wing may be made a South pole, causing the two mating surfaces to be drawn together.

The distal tip and distal extension can be eliminated if desired, and with them, the apertures in the distal wings that accommodate them; the latter can increase the strength of the distal wings. Many other designs for the toothed rail are possible, including those in which the teeth are on an inside surface of a hollow rail (instead of on the outside surface as depicted here) with the catch heads appropriately relocated. Features may be provided on the proximal tip of the device which engage corresponding features at the distal end of the push tube, such that the device can be rotated (e.g., to select the orientation of the wings with respect to the tissue) by rotating the push tube, in lieu of rotating the pull wire as already described. Since the ability of the narrow pull wire to transmit torque is limited; this approach may be quite advantageous. In lieu of a ratcheting mechanism to keep the device in a shortened configuration, other mechanisms may be used, such as a simple threaded rod of the type found in toggle bolts. While it might not be practical to fabricate a sufficiently-smooth helical thread monolithically using a multilayer electrochemical fabrication process, a conventionally-manufactured threaded rod can be assembled together with parts made using EFAB technology to produce a complete device. The use of a threaded rod also provides for continuous adjustability in device length, as opposed to the discrete steps of a toothed rail. A nut which threads onto the rod may also be conventionally manufactured or potentially manufactured via the EFAB technology. The catch housing may be eliminated if the risk of interference with device delivery is not significant. The minimum separation between the tissue contact surfaces of the proximal and distal wings is determined in large part by the length of the catch housing and thus the catch beams. If desired and if the force required to shorten the device is not thereby made too great, the length of the catch beams may be significantly reduced from that shown in the drawings, so as to decrease this minimum separation. If desired for redundancy, to help stabilize the toothed rail within the device, etc., multiple catches may be provided, engaging the rail at different locations. The device can be designed such that the catches are located at the distal end, with the rail moving distally to shorten the device. The device may be built using a multilayer electrochemical fabrication technology in the configuration shown in FIG. 5; however, this takes up a significant amount of space on a wafer. More compact configurations are possible. For example, if a mechanism is provided for releasing the catches, the device can be built with the toothed rail in a more proximal position, then stretched after fabrication to the configuration shown. Perhaps more significantly, the wings can be built in a more closed, or even fully-closed position, if the amount by which they are required to be opened by the springs is less, if the springs ‘launch’ them to a more open position when relaxed, or if the springs can be preloaded (e.g., by using a batch or wafer-scale fixture or process) after fabrication without relying on moving the wings to a closed position post-fabrication to pre-load them springs. If desired for improved visualization during delivery, modifications can be made to the device. For visualization using angiography or other X-ray modalities, the device may incorporate surface or sub-surface (buried) radio-opaque material such as gold in select locations (e.g., the distal and proximal tips, the wing tips) or more globally. For visualization using ultrasound imaging (e.g., intracardiac ultrasound, transesophageal ultrasound, or transthoracic ultrasound), it may be desirable to provide a surface texture (e.g., small depressions) on the surfaces of the device to form an acoustic diffuser that reduces specular reflections and thus blurring of images, as described in the research of Professor Pierre DuPont at Boston University.

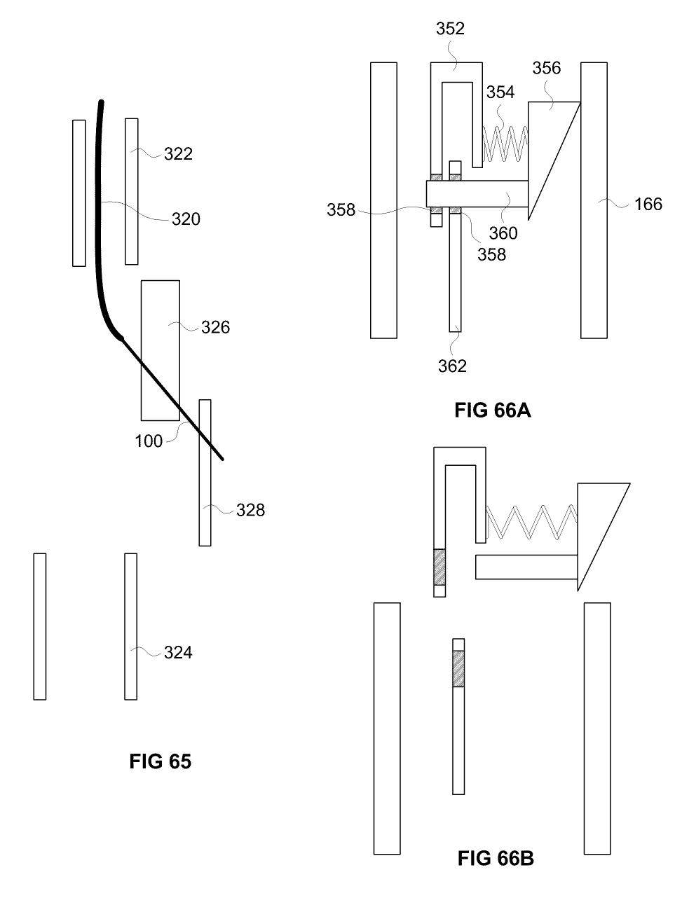

Alternative mechanisms for connecting the rail puller to the rail than those described herein are possible. For example, a mechanism that relies on withdrawal of the push tube 166 and/or needle is possible, as is shown schematically in FIGS. 66A-66B As shown, the rail puller 362 and rail 352 can be attached through rings which are held together by a pin 360 that is attached to a ramp 356 or other shape. A ramp with the narrow end either distal or proximally-oriented allows easy loading of the mechanism into the tube 164. The ramp is displaced outwards by a compression spring 354. In FIG. 66A, the pin 360 engages the rings 358 because the mechanism is inside push tube 164 (and/or needle) and the ramp 356 is pushed inwards, compressing spring 354. In FIG. 66B the push tube 164 (and/or needle) has been withdrawn (e.g., near the end of the delivery procedure) and the ramp 356 has snapped out to relax the spring 354; the pin 360 has now withdrawn from the rings 358 allowing them to separate as shown. The device can be designed in a wide variety of sizes; for example, the span of the wings, the length of the rail, and the length of the catch housing can all be different than in the design shown in the figures. The ramp 356 in some alternatives may have a double slope (i.e. a slope that faces in both the proximal and distal direction along the longitudinal axis of the push tube. Devices that are smaller may be made for more delicate procedures, while large, more robust devices with higher tensile strengths may be made for procedures requiring them.

While the device described herein has been described for procedures which involve approximation and retention of two walls of tissue, clearly the device can approximate and retain multiple tissues if sufficiently long and if all of the tissues are penetrated by the delivery needle. Conversely, the device is useful even for a single walls of tissue; once installed either the distal or proximal end (possibly equipped with specialized features) can be used to secure a patch over a hole (e.g., in hernia repair or atrial or ventricular septal defect repair), or as a binding post or anchor onto which devices and conventional sutures can be attached, etc. Thus references to a tissue wall do not preclude the existence of several walls, and references to walls do not preclude there being only a single wall.

In some cases it may be desirable to install the device in a wall of tissue that is thick enough that the device may need to be impractically long if the device relied on the distal wings spreading beyond the distal surface of the tissue wall. Also in some cases, it may be undesirable to have any portion of the device protrude beyond the most distal surface. In all these cases, other embodiments of the device are possible. For example, the distal and/or proximal wings may be shaped such that when expanded they become anchored within—versus beyond—the wall of the tissue. Such wings may be provided with sharp features and may be expanded either by one or more strong springs or by some other mechanism, in order to adequately penetrate the tissue wall. In one embodiment, forceful opening of the wings may be accomplished against the pressure of the surrounding tissue by a rack and pinion or other mechanism actuated by pulling on the pull wire, or else decoupled from the elements that shorten the device overall, and activated by a separate mechanism, possibly with a separate pull wire. Alternatively, the distal and/or proximal wings may be replaced by a different anchoring mechanism that relies on expansion within tissue, local modification of tissue (e.g., radio frequency-induced contraction of tissue around the device, thermal welding of tissue around the device), etc. In still other alternatives the anchoring mechanism may include one or more fixed barbs which allow motion of the anchor in a distal direction but restrain it in a proximal direction. In still other embodiments, the proximal end of the device, as opposed to the distal end, may be embedded in tissue as opposed to having it extend from the proximal end of the proximal tissue wall. In still other embodiments, both distal and proximal ends may be embedded in tissue.

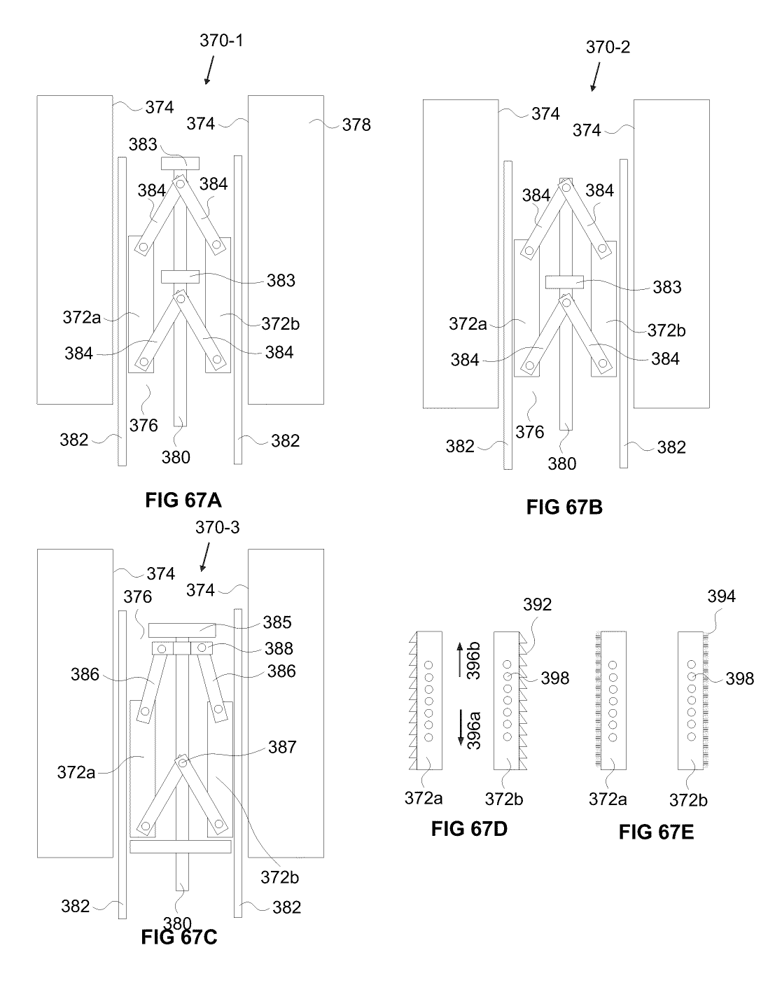

Examples of some of these alternatives are provided in FIGS. 67A-67C. These figures provide side views illustrating three alternative tissue anchor mechanisms 370-1, 370-2, and 370-3 respectively that use anchoring shoes 372a and 372b that push up against interior sides 374 of an opening 376 in tissue 378. Shoes 372a and 372b may be forced to separate by pulling tensioning element 380 while holding a push tube or other immolation device in a fixed position after a needle 382 or other delivery device is withdrawn. As tensioning element 380 is pulled wings 384 and 386 rotate outward forcing shoes 372a-372b into deeper contact with sides 374 of tissue 378. In some embodiments, after withdrawal of the delivery needle, wings 384 and 386 may spring open slightly to cause initial engagement between shoes 372a and 372b and tissue 378. Shoes may have smooth surfaces as shown in FIGS. 66A-66C or they may have roughened surfaces 392 or even spiked surfaces 394 that tend to lock the shoes to the tissue. The surface may be roughened to cause bi-directional locking or they may be roughened to in a non-symmetric manner to allow unidirectional locking (such as that shown in FIG. 66D where shoe slippage relative to tissue in direction 396B may be more likely than in direction 396A). Shoes 372A and 372B may also contain one or more holes 398 or surface perturbations that enhance likelihood of tissue in growth to enhance locking. As seen in FIGS. 67A-67C, wings 384 may be attached to tensioning element 380 via pivot pins 387. Wings 386 may also be attached to tension elements via slides 388. Stops 383 and 385 may be used to limit the allowable amount of rotation that the wings can undergo. In some embodiments, the wings may be allowed to rotate (i.e. the stops configured to allow rotation) to a perpendicular orientation relative to the longitudinal axis of the device while in other embodiments less rotation or even greater rotation may be allowed. In some embodiments, rotation somewhat (e.g. 5-20 degrees) beyond the perpendicular may be advantageous to cause locking of the shoes in an opened state. As can be seen in FIG. 67C some embodiments may have distal and proximal wings of different lengths. Such difference may allow the shape of the outer surfaces of the open shoes to take on a parallel orientation relative to the insertion direction of the device into the tissue, greater spacing along the insertion direction into the tissue, or smaller spacing along the insertion direction into the tissue.

In some cases it may be desirable for the device to be non-permanently installed within the body. In one embodiment the device may be fabricated from a material (e.g., particular polymers, or a suitable magnesium alloy) that can be resorbed by the body. Polymers (whether resorbable or not) may be molded (e.g., by injection molding) to form either the entire device monolithically (possibly requiring a sacrificial mold to release the molded part), or the device can be fabricated monolithically using a layered manufacturing/solid freeform fabrication process that builds structures from resorbable polymers, or components of the device can be molded discretely or in subassemblies, which are then assembled. In another embodiment only certain portions of the device (e.g., the wings) are made from resorbable material, thus allowing removal of the remainder of the device once these portions have resorbed. In an alternative embodiment, the device may be entirely fabricated from a permanent material, but removed from the body by a mechanism (built-in to the device and/or externally applied) which allows the toothed rail to be released from the catches in order to lengthen the device, and moves the wings (distal, proximal, or both) to a sufficiently-closed position that withdrawal of the entire device from the tissue is possible. In one embodiment, the toothed rail may be disengaged from the catches by displacing the former with respect to the latter in a direction perpendicular to the longitudinal axis of the rail, such that the catches ‘miss’ the teeth.

It is desirable when delivering the device to know how the needle must be advanced through the tissue to ensure that the distal wings, once released, will be able to freely expand. In one embodiment a mechanism is provided to assist with this aspect of delivery. For example, the delivery needle may include a slot in its side through which an probe-like element (e.g., ramp-shaped to allow it to be pulled back through the tissue when the needle is withdrawn) located at the appropriate distance from the needle tip protrudes when a spring attached to it relaxes and there is space around the needle available. When the needle has sufficiently advanced such that the element clears the distal tissue wall, the element protrudes and through mechanical (e.g., releasing a wire that the physician keeps under slight tension) or electronic/electromechanical means, signals the physician (or automated apparatus used for device delivery) to stop advancing the needle. In one embodiment, rather than signal, the element can release an interlock that allows the needle to be withdrawn (from around the device); thus the physician can advance the needle to a position based on his best knowledge, and be assured that when the needle is withdrawn the device will not be exposed unless the distal wings have sufficient room to open distally.

In one embodiment of the device, an interlock is provided such that the device cannot be shortened unless the wings have been adequately extended, since delivering a device under these conditions may result in it extruding through the perforation. When the physician pulls the pull wire to shorten the device, the abnormal resistance offered to motion then serves as an indicator that the device is not properly deployed.

In one embodiment of the device, an interlock is provided which prevents inadvertent shortening until the device is installed within the delivery needle, thus avoiding a possible situation in which the device is not as long as expected and this is only discovered during the delivery process.

In one embodiment of the device, the wings can open in other directions than that shown in the figures (i.e., the distal wings opening distally and the proximal ends opening proximally). For example, the distal wings may open proximally, so long as a means (e.g. a mechanical stop) is provided to prevent the wings over-traveling and ending up at an angle that does not provide a sufficiently-large overlap area with the tissue wall. In other embodiment of the device, the wings may open without significant rotation, for example, by moving linearly, perpendicular to the longitudinal axis of the device.

If desired, the rail puller, once disconnected, can be reconnected to the rail in order to tighten the device after it has been delivered. For example, if multiple devices are delivered to the same region of tissue, it may be advantageous (e.g., to reduce stress on the device or the tissue, the latter of which may cause the device to pull out) to initially leave all of them loose, and then tighten them gradually, a little at a time in alternation. In one embodiment, the interface between rail and rail puller is specially designed to facilitate re-attachment. Alternatively, another instrument (e.g., forceps or a custom-designed instrument) may be used to pull on the rail to tighten the device. The proximal end of the rail can be specially designed to facilitate grasping with such an instrument. Atrial septal defects and ventricular septal defects in the heart that are too large to close without the use of a patch due to the high stress on the tissue caused by the large displacement required, might be closed without a patch using devices that allow gradual tightening.

Automated, semi-automated, or manually-operated motorized apparatus can be provided, for example, to execute the motions shown in FIGS. 56-57, FIG. 58 or 59, and FIGS. 60-62. In one embodiment, a handheld system consists of a handheld motorized unit coupled to a delivery system (fairly short for open procedures, or long for minimally-invasive procedures). In the case of an automated or semi-automated system, the physician can then approximate and retain tissue by merely poking the delivery needle through the tissue and pressing a button that initiates the sequence of motions.

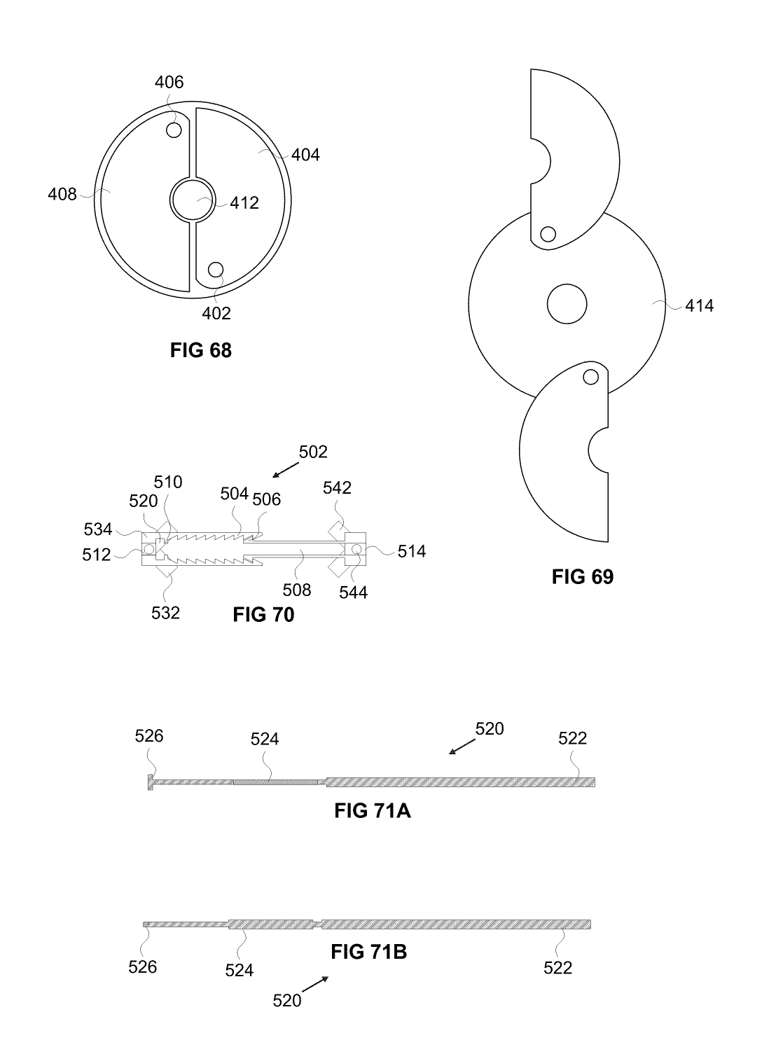

An alternative wing configuration is illustrated with the aid of FIGS. 68 and 69. Instead of toggles swinging open along axes which are perpendicular to the axis of the insertion shaft (i.e. perpendicular to the longitudinal axis of the instrument), the device of FIGS. 68 and 69 includes wings that pivot open along axes that are substantially parallel to the axis of the shaft (i.e. parallel to the longitudinal axis of the instrument). During introduction to the tissue wall, the device is preferably inserted without a rotation along its axis so that the wings stay in their retracted position. After insertion the device is rotated (e.g. counterclockwise in the illustrated embodiment) so that the wings spread out so as to define a larger area, with the wings overlapping a region of the tissue wall such that the distal end of the device cannot be extracted from the tissue in the direction opposite to the direction of insertion. The wings are retained in an open position while seating of the wings onto the tissue surface occurs. In some alternative embodiments more than two wings may exist. In other embodiments, the end of each initial wing element may have another pivot axis from which one or more secondary wings may extend. The extension of the wing elements may be limited by stops or other elements (not shown). In still other embodiments, the wings may be perforated to allow tissue growth to extend through the wings to help form a permanent attachment. In some other embodiments, the wings may be designed to ratchet open so that once opened they will not readily close or at least not close without activation of a secondary mechanism. In still other embodiments, instead of relying on rotational acceleration to swing the arms open, gearing may exist between the pivot access of the wings and the central shaft such that rotation of the central axis causes the outward (or possibly) inward pivoting of the wings (not shown). In still other embodiments, the wings may be formed in an open position and then compressed to a closed position against spring elements that are formed along with the retention element and loaded into a delivery tube, catheter, or needle. The wings may be closed prior to seating them against tissue, for example, by rotating the device counterclockwise and stopping the rotation so that the inertia of the wings swings them closed. Upon removal from the delivery tube the wings may spread out under the influence of the compressed spring elements.

Wings of the type shown in FIGS. 68 and 69 may be used at either end of a device (the distal end or the proximal end). Alternatively, one end of the device may use this type of wing, while the other end uses another type of wing (e.g., the type shown in FIG. 6). Both ends of the device may be brought together in one of the manners discussed herein above or in some alternative manner.

In some alternative embodiments, instead of the wings moving from a retracted position to an expanded (or deployed position) via rotating around pivots as described above, wings may be of a shape and material that allow them to be compressed into a configuration that enables them to be passed through the tissue wall(s) while inside a needle or other tube. Once this is done, withdrawal of the needle may allow the wings to simply spring, snap, or ‘pop’ into final shape. In some cases, a superelastic material may be used to provide the required functionality while in other cases, spring structures may be formed along with the device and then comprised when loaded into a needle.

In other alternative embodiments, the catch heads and rail puller may take on other forms. An example of this is illustrated in the plan views of FIGS. 70-71B. In these figures the various elements are not shown to scale. FIG. 70 provides a plan view of a single device 502 in which the teeth 504 that engage the catch heads 506 are on the inside surface of the distal portion of the device 502, and the catches on the catch head face outward to engage them. A channel 508 large enough to accommodate the rail puller 520 shown in FIGS. 71A and 71B runs down the longitudinal axis of the device, giving rise to a proximal aperture 514 and a distal aperture 512. In FIGS. 71A and 71B, the rail puller 520 is shown from the top and from the side respectively. The puller has a proximal widening 522 that may extend both side-to-side and top-to bottom, as shown, as well as a distal widening 524 that only extends only top-to-bottom. The puller 520 also has a lug 526 (e.g., at its distal end) which only extends side-to-side (i.e., at 90 degrees to the distal widening). Other than the lug 526, portions of the puller 520 can pass entirely through the channel in the device; the lug 526 can only pass through the channel when the puller is rotated such that the lug clears the lug shelf 510 which forms the proximal end of the rail puller interface 518. As in some of the prior embodiments, the device includes distal wings 532, proximal wings 542, distal wing pivots 534, proximal wing pivots 544.

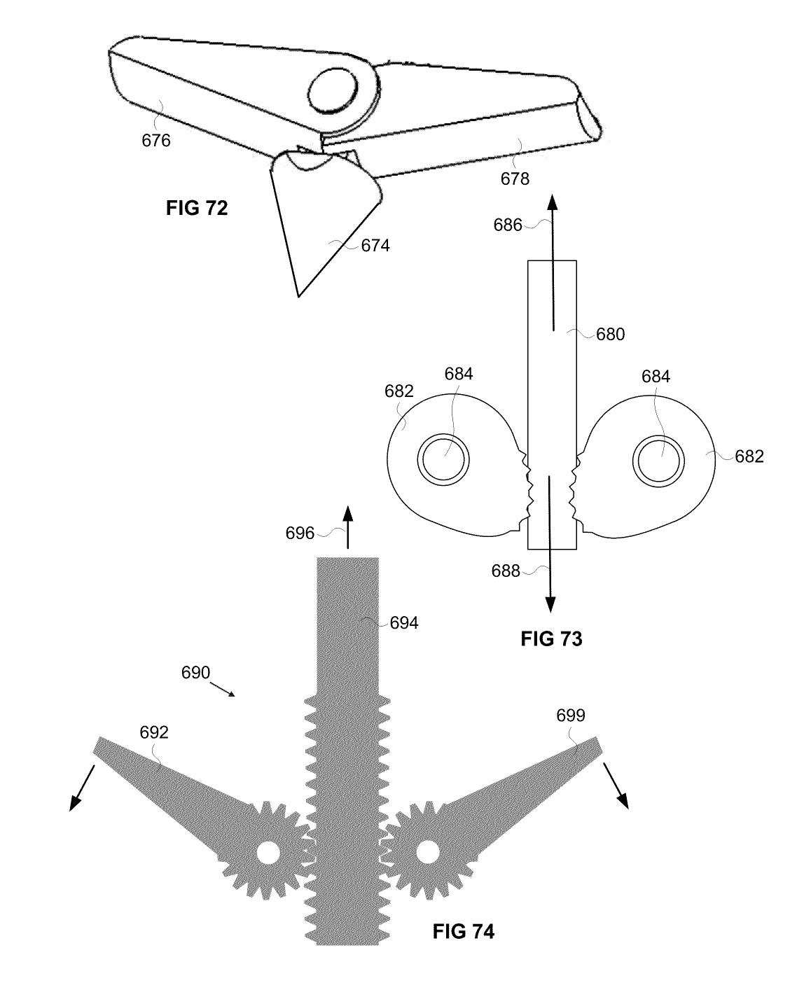

In some alternative embodiments, in lieu of delivering an approximation device through a needle which perforates the tissue walls and introduces the device, the distal end of the distal tip 674 of a device, for example having distal wings 676 and 678, may be made sharp (e.g., like a trocar), as shown in FIG. 72. In such embodiments, the device itself may be able to penetrate the walls without a needle when appropriate force is applied. In such embodiments, the tip is preferably equal to or greater in width at its proximal end, than the distal end of the distal folded wings, so that the latter are unlikely to catch on the proximal surface of the tissue walls during delivery. If no needle is provided to keep the wings closed, the distal wings may be open or partially open initially, but forced to close at least partially as the device is inserted through the wall. Once clearing the distal surface of the tissue, they would then spring at least partially open as already described.

In some alternative embodiments, a sharp distal tip may present a risk of tissue damage, etc., as such some such embodiments may include a mechanism that effectively blunts the tip after it penetrates the walls. It is preferred, though not necessary, that the mechanism for blunting the tip be associated with the opening of the wings. For example, the tip may be formed by extensions from the wings, such that rotation of the opening wings serves to move the extensions to a position where they no longer form a sharp tip. In another embodiment, the tip itself may be blunt, but the distal end is surrounded by a relatively short sharp tube or needle which retracts away from the distal end of the distal tip by the time device delivery has been completed; this tube may remain a part of the delivered device, unlike the delivery needle described earlier. In still other alternative embodiments, the distal wings may not only pivot open but be capable of sliding along the longitudinal axis of the device toward and over or partially over the tip during tissue approximation, thus allowing an interior portion of the wings to cover the sharp tip after the wings have fully opened.

In some embodiments, instead of using a needle to deliver the device or making the distal tip sharp so it can penetrate tissue, one can create a hole in the tissue wall using a separate instrument (e.g., a trocar or needle), then install the device through the hole. In this case, the device may be held within a tube (which may be blunt) or another mechanism may be provided if it is desired to keep the wings in a closed position.

In some embodiments instead of using a toothed rail to connect the distal and proximal wings, along with catches to prevent motion in the direction that increases the longitudinal dimension of the device, one or more miniature rotating cleats of the sort used to hold in place the ropes on sailboats can be provided. A pair of such cleats is illustrated in FIG. 73. The cleats 682 may rotate around pivots 684 to allow a length of material 680 that is preferably textured or has soft surface (e.g., a metal shaft or suture material) such that relative motion with respect to the cleat is allowed in the proximal direction but restrained in the distal direction 686 and permitted in the proximal direction 688, as shown.

In some embodiments, the delivery needle may comprises one or more joints, either single-axis or multiple-axis. This may allow the angle and/or position of the needle to be changed to facilitate access of the device to the desired tissue region, or to provide a preferred angle for the needle to enter the tissue.

In some embodiments, a tension-limiting clutch may provided to allow the device to gradually elongate (e.g., if the tissue grows). Such a clutch may allow some motion to occur once the tension applied to the device reaches a threshold. The clutch may be based on frictional effects, or the like, or may simply comprise a properly-sized material which undergoes plastic deformation at a particular stress (preferably well below its ultimate tensile strength).

In some embodiments, the wings of the device may preferably be of a different shape, or extended to a different angle with respect to one another than discussed previously, such that the tissue contact surfaces are adapted to engage tissue or devices of different geometries. For example, the wings may be extended to a larger angle than 180°, or to an angle smaller than 180°. In particular, if the angle is less than 180 degrees (i.e. the wings form a “V” shape) the device may be useful for securing tissue or devices with circular or elliptical cross sections; examples of such tissue include blood vessels and the ureters. Examples of devices that may be secured include annuloplasty rings that are normally sutured to the interior of the heart to alter the shape of a valve, such as the mitral valve. In some embodiments, the shape and/or degree of extension of the proximal and distal wings may be different. For example, in the case of securing an annuloplasty ring, the distal wings of the device may open to approximately 180° to optimally anchor behind a wall of tissue, whereas the proximal wings which hold the ring to the tissue wall may open to a smaller angle (e.g., 90°), forming a “V” that captures the ring and prevents it from sliding.

In some embodiments, the wings may be extended actively, by means such as gears or linkages. This can be particularly useful if the wings might otherwise have some difficulty extending. One example is anchoring the device within a relatively solid mass of tissue, versus against a wall of tissue (by extending the wings against the wall as has been previously described). The distinction is that of forming a blind hole in the tissue for anchoring, versus a through-hole. Anchoring at least one end (typically the distal end) of the device in solid tissue may be advantageous in some applications (e.g., to avoid a very long device when the distance to the nearest wall is significant), or even necessary (e.g., to avoid a portion of the device protruding beyond the tissue).

FIG. 74 shows a wing design using a rack-and-pinion mechanism 690 to extend the wings 692 (shown at least partially extended), causing them to dig into the tissue, as a central shaft 694 is pulled along direction 696. In such a design, the more tension that is applied to the shaft, the more the wings extend and dig into the tissue (as long as the wings are prevented from overextending beyond the position where they are roughly parallel). In still other embodiments, barbed wings designed to anchor the device within a mass of tissue may be extended by self-expanding means, such as springs, e.g. those made of superelastic materials such as nickel-titanium. In some embodiments, the type of wing used at the proximal and distal ends of the device may be different; for example, wings of the type shown in FIG. 74 may be used at the distal end and those of the type shown in FIG. 5 may be used at the proximal end.

In some embodiments, the device may be provided with a single wing in lieu of two or more as described. This wing may be asymmetrically located with respect to the main body of the device, such that it extends substantially to one side of the device when extended. Alternatively, the wing may be designed to rotate about a more central point such that the wing extends somewhat symmetrically on opposite sides of the device. As with some wings already described, springs may be provided to at least partially extend the wings, and contact between the wing and the tissue may assist in extending the wings.

As has already been discussed with regard to FIG. 73, in some embodiments, the toothed rail may be replaced by another structure with sufficient tensile strength. For example, a standard suture material may be used.

In some embodiments, methods other than rotation of the rail puller, as has already been described, may be used to detach the rail puller or pull wire from the device after delivery of the latter. Mechanisms which require an alternative motion of the pull wire (e.g., advancing it without the need to rotate it) might be provided. Alternatively, materials with variable mechanical strength may be used as means of attachment. For example, the wire or puller may be joined to the device with a dissolvable material, including materials that may be electrolytically dissolved such as solder (as with Guglielmi detachable coils used in treating brain aneurysms), thermoplastic materials such as solder and polymers, and other materials.

In some alternative embodiments, in lieu of delivering an approximation device through a needle which perforates the tissue walls and introduces the device, the distal end of the distal tip 674 of a device, for example having distal wings 676 and 678, may be made sharp (e.g., like a trocar), as shown in FIG. 72. In such embodiments, the device itself may be able to penetrate the walls without a needle when appropriate force is applied. In such embodiments, the tip is preferably equal to or greater in width at its proximal end, than the distal end of the distal folded wings, so that the latter are unlikely to catch on the proximal surface of the tissue walls during delivery. If no needle is provided to keep the wings closed, the distal wings may be open or partially open initially, but forced to close at least partially as the device is inserted through the wall. Once clearing the distal surface of the tissue, they would then spring at least partially open as already described.

In some alternative embodiments, a sharp distal tip may present a risk of tissue damage, etc., as such some such embodiments may include a mechanism that effectively blunts the tip after it penetrates the walls. It is preferred, though not necessary, that the mechanism for blunting the tip be associated with the opening of the wings. For example, the tip may be formed by extensions from the wings, such that rotation of the opening wings serves to move the extensions to a position where they no longer form a sharp tip. In another embodiment, the tip itself may be blunt, but the distal end is surrounded by a relatively short sharp tube or needle which retracts away from the distal end of the distal tip by the time device delivery has been completed; this tube may remain a part of the delivered device, unlike the delivery needle described earlier. In still other alternative embodiments, the distal wings may not only pivot open but be capable of sliding along the longitudinal axis of the device toward and over or partially over the tip during tissue approximation, thus allowing an interior portion of the wings to cover the sharp tip after the wings have fully opened.

In some embodiments, instead of using a needle to deliver the device or making the distal tip sharp so it can penetrate tissue, one can create a hole in the tissue wall using a separate instrument (e.g., a trocar or needle), then install the device through the hole. In this case, the device may be held within a tube (which may be blunt) or another mechanism may be provided if it is desired to keep the wings in a closed position.

In some embodiments instead of using a toothed rail to connect the distal and proximal wings, along with catches to prevent motion in the direction that increases the longitudinal dimension of the device, one or more miniature rotating cleats of the sort used to hold in place the ropes on sailboats can be provided. A pair of such cleats is illustrated in FIG. 73. The cleats 682 may rotate around pivots 684 to allow a length of material 680 that is preferably textured or has soft surface (e.g., a metal shaft or suture material) such that relative motion with respect to the cleat is allowed in the proximal direction but restrained in the distal direction 686 and permitted in the proximal direction 688, as shown.

In some embodiments, the delivery needle may comprises one or more joints, either single-axis or multiple-axis. This may allow the angle and/or position of the needle to be changed to facilitate access of the device to the desired tissue region, or to provide a preferred angle for the needle to enter the tissue.

In some embodiments, a tension-limiting clutch may provided to allow the device to gradually elongate (e.g., if the tissue grows). Such a clutch may allow some motion to occur once the tension applied to the device reaches a threshold. The clutch may be based on frictional effects, or the like, or may simply comprise a properly-sized material which undergoes plastic deformation at a particular stress (preferably well below its ultimate tensile strength).

In some embodiments, the wings of the device may preferably be of a different shape, or extended to a different angle with respect to one another than discussed previously, such that the tissue contact surfaces are adapted to engage tissue or devices of different geometries. For example, the wings may be extended to a larger angle than 180°, or to an angle smaller than 180°. In particular, if the angle is less than 180 degrees (i.e. the wings form a “V” shape) the device may be useful for securing tissue or devices with circular or elliptical cross sections; examples of such tissue include blood vessels and the ureters. Examples of devices that may be secured include annuloplasty rings that are normally sutured to the interior of the heart to alter the shape of a valve, such as the mitral valve. In some embodiments, the shape and/or degree of extension of the proximal and distal wings may be different. For example, in the case of securing an annuloplasty ring, the distal wings of the device may open to approximately 180° to optimally anchor behind a wall of tissue, whereas the proximal wings which hold the ring to the tissue wall may open to a smaller angle (e.g., 90°), forming a “V” that captures the ring and prevents it from sliding.

In some embodiments, the wings may be extended actively, by means such as gears or linkages. This can be particularly useful if the wings might otherwise have some difficulty extending. One example is anchoring the device within a relatively solid mass of tissue, versus against a wall of tissue (by extending the wings against the wall as has been previously described). The distinction is that of forming a blind hole in the tissue for anchoring, versus a through-hole. Anchoring at least one end (typically the distal end) of the device in solid tissue may be advantageous in some applications (e.g., to avoid a very long device when the distance to the nearest wall is significant), or even necessary (e.g., to avoid a portion of the device protruding beyond the tissue).

FIG. 74 shows a wing design using a rack-and-pinion mechanism 690 to extend the wings 692(shown at least partially extended), causing them to dig into the tissue, as a central shaft 694 is pulled along direction 696. In such a design, the more tension that is applied to the shaft, the more the wings extend and dig into the tissue (as long as the wings are prevented from overextending beyond the position where they are roughly parallel). In still other embodiments, barbed wings designed to anchor the device within a mass of tissue may be extended by self-expanding means, such as springs, e.g. those made of superelastic materials such as nickel-titanium. In some embodiments, the type of wing used at the proximal and distal ends of the device may be different; for example, wings of the type shown in FIG. 74 may be used at the distal end and those of the type shown in FIG. 5 may be used at the proximal end.

In some embodiments, the device may be provided with a single wing in lieu of two or more as described. This wing may be asymmetrically located with respect to the main body of the device, such that it extends substantially to one side of the device when extended. Alternatively, the wing may be designed to rotate about a more central point such that the wing extends somewhat symmetrically on opposite sides of the device. As with some wings already described, springs may be provided to at least partially extend the wings, and contact between the wing and the tissue may assist in extending the wings.

As has already been discussed with regard to FIG. 73, in some embodiments, the toothed rail may be replaced by another structure with sufficient tensile strength. For example, a standard suture material may be used.

In some embodiments, methods other than rotation of the rail puller, as has already been described, may be used to detach the rail puller or pull wire from the device after delivery of the latter. Mechanisms which require an alternative motion of the pull wire (e.g., advancing it without the need to rotate it) might be provided. Alternatively, materials with variable mechanical strength may be used as means of attachment. For example, the wire or puller may be joined to the device with a dissolvable material, including materials that may be electrolytically dissolved such as solder (as with Guglielmi detachable coils used in treating brain aneurysms), thermoplastic materials such as solder and polymers, and other materials.

First Group of Embodiments—Delivery of Multiple Devices:

In some circumstances, it may be desirable to deliver multiple devices simultaneously or in rapid succession to multiple locations in the patient's body. In some embodiments intended for such delivery, the system includes a group of delivery systems of a type that can deliver one device at a time. In some embodiments, these systems may be loosely coupled together, to allow each device to be delivered somewhat independent of the position of others within a region of the body. In other embodiments, the systems are more rigidly coupled such that devices are delivered in a particular spatial relationship without the need to individually steer each delivery system to its target location. In these embodiments, the delivery systems may share elements (e.g., push tubes, pull wires, or needles), or have elements that are ganged together, so as to move together.

Multiple devices may be placed in a single delivery system, one at a time, for successive delivery, without the need to withdraw the delivery system from the patient each time, by virtue of the fact that devices may be loaded into the delivery system either from its distal end, or in this case, its proximal end. Reloading of the delivery system can be accomplished by pulling out the push tube, loading a device, replacing the push tube, and using it to push the device distally (e.g. toward the distal end of the guiding catheter). In some embodiments that avoid having to remove the push tube to load a device, the devices have continuous channels from end to end, and the push tube is small enough that it can pass through these channels. Pushing of devices may be accomplished, for example, using a spring-loaded catch on the distal end of the push tube (or on the proximal end of the device) which engages a device when the latter is correctly positioned at the distal end of the push tube. This catch allows distally-directed motion of the device with respect to the push tube, but not proximally-directed motion once the device has reached the distal end of the tube. Multiple devices can be loaded into the push tube and pushed down to the distal end (where the push tube engages them). This loading may occur, for example, via another pushing device (such as a wire), by inertial forces (e.g., a whipping motion), by gravitational forces, by magnetically dragging the device using a magnet outside the delivery system walls, or the like.

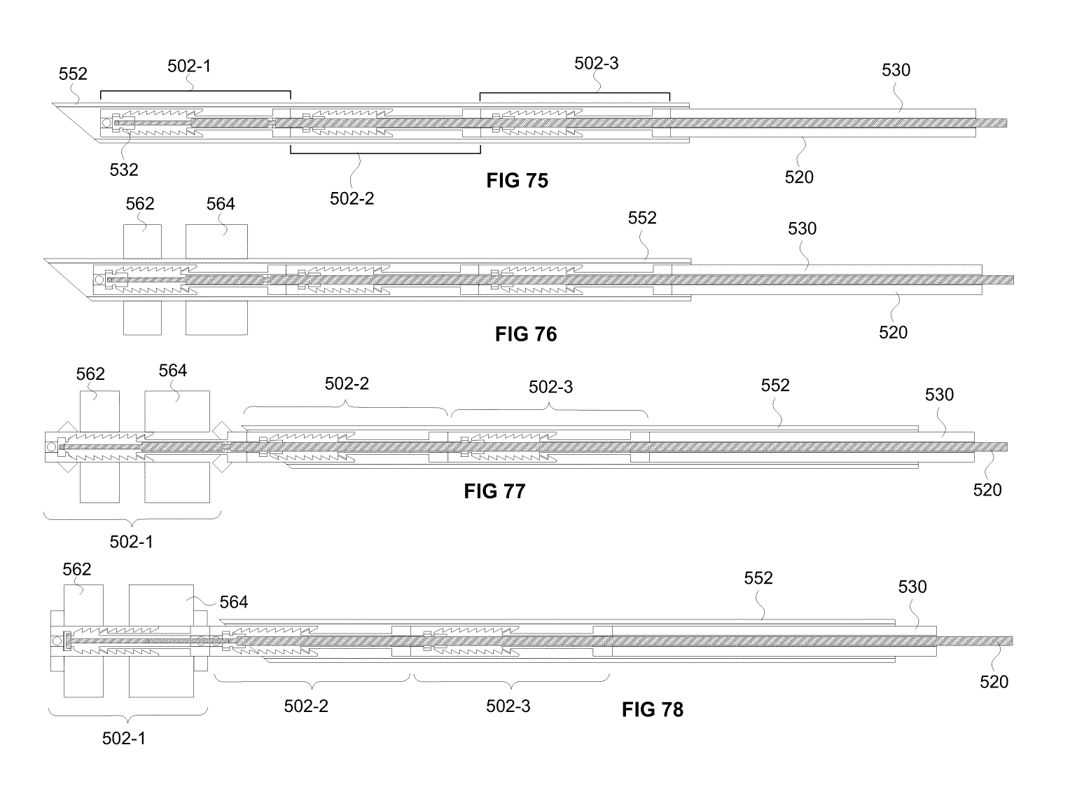

In some embodiments, multiple devices may be placed in a single needle, or associated catheter, simultaneously in an end-to-end (i.e., in tandem) fashion, and delivered one after another, in some cases very quickly. An example of this is illustrated in the plan views of FIGS. 75-81. In these figures the various elements are not shown to scale. In some variations of these multiple device dispensing instruments, the configuration of the teeth, catch heads, and rail puller may follow the alternatives of FIGS. 70-71B. As in some of the prior embodiments, the device includes distal wings 532, proximal wings 542, distal wing pivots 534, proximal wing pivots 544.

In FIG. 75, three devices 502-1, 502-2, and 502-3 are shown installed in a needle 552. In some embodiments, many more than three devices may be load into the needle. Along with the needles and devices, a rail puller 520 is shown along with push tube 530. The rail puller 520 is long enough to reach the most distal device, and the push tube 530 bears against the proximal end of the most proximal device 502-3. In some embodiments, the more proximal portions of the rail puller may be replaced with a wire or cable that is able to transmit tension and torque to its distal portions.

In FIG. 76, the needle of FIG. 75 is shown has having pierced a proximal tissue wall 564 and distal tissue wall 562 that are to be approximated.

FIG. 77, depicts the state of the device delivery process after the needle has been partially withdrawn. This withdrawal has occurred while holding the push tube in a fixed position so that the wings of the first device 502-1 are fully exposed on both the proximal and distal sides of the proximal tissue wall 564 and distal tissue wall 562, respectively. At this point in the process, the wings have partially opened.

Unlike previous figures, here the tissue of the proximal and distal walls is shown to have recoiled, leaving a smaller hole once the needle was removed. By virtue of the distal widening of the rail puller the inward deflection of the catch heads has been prevented and thus device 502-1 was prevented from shortening while the needle was being withdrawn. Such shortening might otherwise occur, if the frictional forces acting between the device and the needle are able to drag the distal end of the device proximally as the needle is retracted.

In FIG. 78, the state of the delivery and approximation process is shown after the rail puller 520 has been pulled while the push tube 530 has been pushed, causing the device to shorten and the wings to open fully and the distal wings 532 to engage the distal wall 562 and the proximal wings 542 to engage the proximal wall 564. By virtue of the proximal widening of the puller inward deflection of the catch heads of device 502-2, device 502-2 is not able to shorten, thus allowing the pushing force of the push tube to be transmitted to device 502-1 as desired, without risk of itself prematurely shortening device 502-2, device 502-3, and any other devices in the stack.

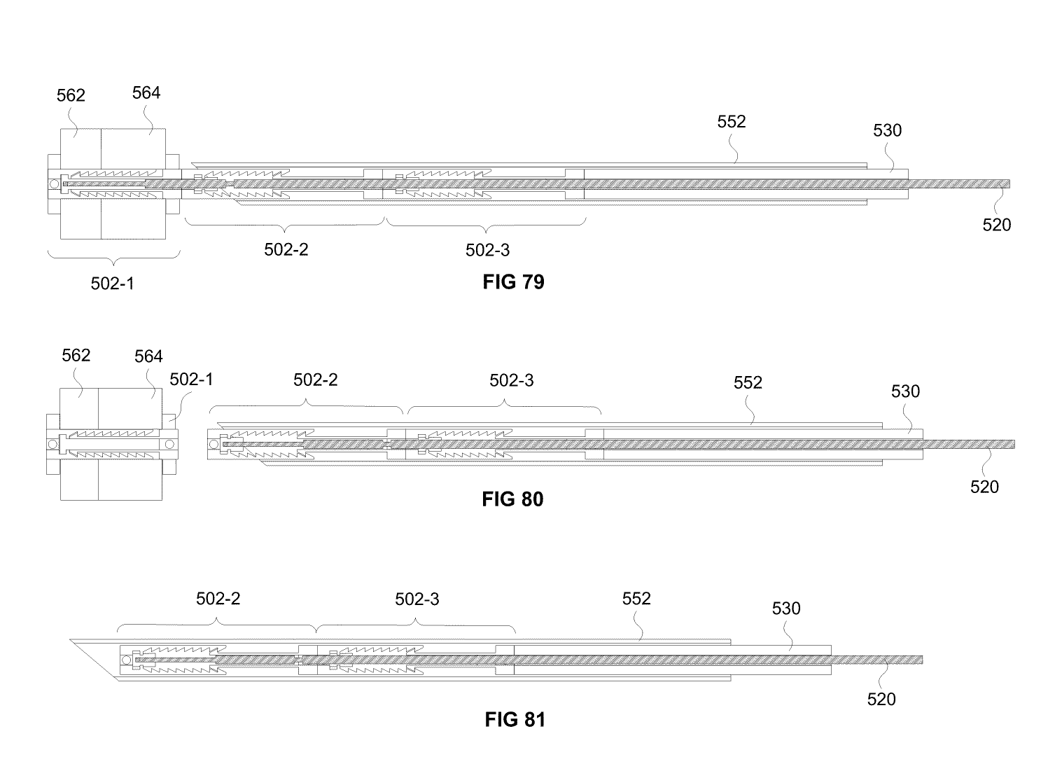

In FIG. 79, the state of the delivery and approximation process is shown after the rail puller has again been pulled while the push tube has been pushed. This additional pulling and pushing brings the distal tissue wall 562 and proximal tissue walls together. Again device 502-2, and the other devices in the stack cannot shorten due to the proximal widening of the puller.

In FIG. 80, the state of the delivery and approximation process is shown after (1) the puller has been rotated approximately 90 degrees such that the lug 526 clears the lug shelf 510 so that it may be disengaged from the rail puller interface on device 502-1 and (2) the rail puller has been pulled entirely out of device 502-1 and device 502-1 is decoupled from device 502-2. As shown in FIG. 80, device 502-1 has been fully delivered.

In FIG. 81, the state of the process is shown after the needle has been advanced to extend past the distal tip of device 502-2 and the rail puller 520 has been made to engage the rail puller interface 518 of device 502-2. As shown in FIG. 84, device 502-2 is now situated similarly to device 502-1 in FIG. 78 and thus the system is ready for delivering device 502-2.

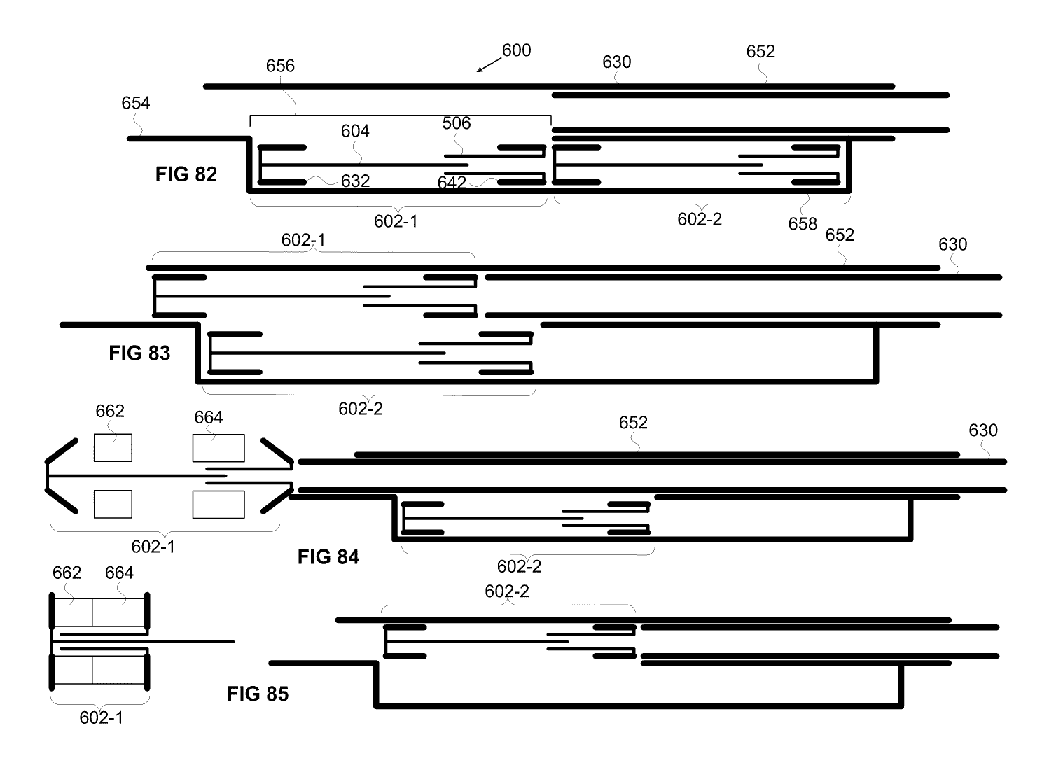

Another approach to delivering multiple devices 602 involves a delivery system 600 of the type shown in the schematic, not-to-scale, cross sectional drawings of FIGS. 82-85. The delivery system 600 uses a modified needle 652 having a tip 654, a side port 656 interfacing with a ‘magazine’ 658 of similar inner diameter which is attached to it and which runs parallel to it. Within the magazine are multiple devices arranged in tandem (end-to-end). The devices 602-1 and 602-2 (others may exist but are not shown) have rails 604 with outward pointing teeth, much like those illustrated in the example of FIG. 5; however, alternative designs (e.g. such as that shown in FIG. 70) may be used. A push tube 530 is also provided. In practice, the portion of the needle distal to the magazine is preferably longer than that shown in the FIGS. 82-85 to enable the needle to penetrate the proximal and distal tissue walls that are to be approximated without interference from the magazine. In some alternative embodiments, the magazine may have sloped distal and proximal ends.

In FIG. 82, two devices are shown in the delivery system, but many more can be provided in practice. Device 602-1 is in the ‘ready’ position, i.e. located adjacent to side port 656, from which it can be transferred to the needle. Device 602-2 is held in reserve. In practice, at the time of loading the needle into a delivery catheter or other delivery system, a first device 602 may already be located in the chamber of the needle thus eliminating the need to withdraw an initial device from the magazine.

In FIG. 83, a mechanism (e.g., comprising a spring, a second push tube, magnet, air or fluid pressure, vacuum, or the like) not shown in the drawing has moved device 602-1 into the main chamber of the needle 652, while another mechanism (or part of the same mechanism) not shown in the drawing has moved device 602-2 into the ready position.

In FIG. 84, the state of the delivery and approximation process is shown after (1) the needle has passed through the proximal tissue wall 664 and the distal tissue wall 662 and (2) the push tube 630 has held device 602-1 in place (i.e. with its distal end beyond the distal end of the distal tissue wall 662 and its proximal end on the proximal side of the proximal tissue wall 664) while the needle was withdrawn. At this point in the process, the device 602-1 is has been delivered to the tissue that is to be approximated has been partially opened but the approximation of the tissue has not yet occurred.

In FIG. 85, the state of the process is shown after device 602-1 has been completely delivered and the tissue approximated and retained. The delivery system is also shown has having been withdrawn and the push tube withdrawn within the needle beyond the side port and device 602-2 has entered the needle from the magazine. At this point in the process, system is ready to deliver device 602-2.

In some alternative embodiments (not shown) of the system shown in FIGS. 82-85, the devices may be arranged in the magazine side-by-side, instead of end-to-end. Such an arrangement may allow the same mechanism that loads successive devices into the needle to advance the successive devices in the magazine to the ready position.

Second Group of Embodiments—Edge-to-Edge Approximation with Winged Structures Located on Entry Side Approximated Tissue:

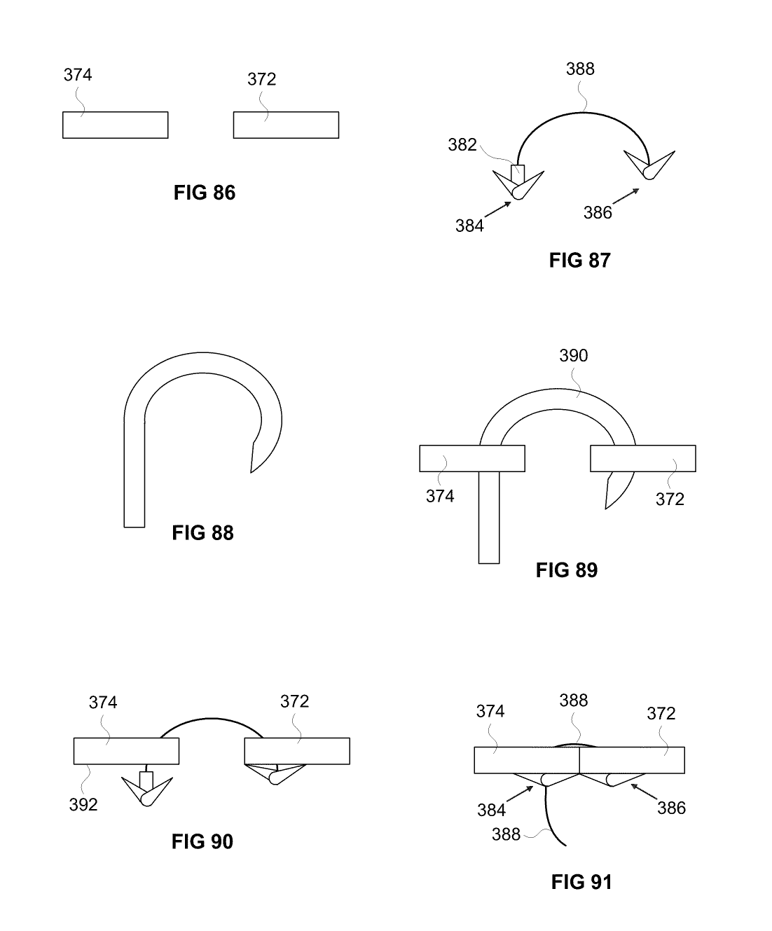

In many cases there is a need to approximate and retain tissue walls 374 (proximal) and 372 (distal) that are side by side as shown in FIG. 86, instead of back to back (i.e., overlapping) as has been discussed herein above. An example of such a case is in the percutaneous repair of valve leaflets which would otherwise need to be sutured in an open procedure. In some cases overlapping of the leaflets may be possible for purposes of repair. An embodiment of the invention for side to side closure is illustrated with the aid of FIGS. 87-91. FIG. 87 illustrates and instrument having a flexible toothed rail 388 along with (e.g. made from a series of articulated links (such as a chain), or is made of a material (e.g., polymer) that is thin enough and/or of low enough modulus to be readily bent at least along one axis), a catch housing 382 located near the proximal end of the instrument along with proximal wings 384 and distal wings 388. For example, the toothed rail shown in FIG. 46 may be made flexible along an axis parallel to the crossbars by deleting the crossbars at both ends of the “I” beam. The device may be delivered through a curved hollow needle 390 as shown in FIG. 88. The delivery procedure shown in the sequence of FIGS. 89-91 (FIG. 89 insertion of the needle that contains the instrument, FIG. 90 deployment of the instrument and withdrawal of the needle, and FIG. 91 bringing the distal and proximal ends of the instrument together to approximate the tissue. This process results in the wings making contact with the same side of the each element of tissue, after which pulling on the rail draws the elements of tissue together. The protruding section of toothed rail may be removed. If made from links, the links may be disconnected from the remainder of the chain. If the rail is made of a continuous material, the protruding part may be cut or snapped off by bending (to facilitate this, scoring indentations may be provided at intervals to concentrate the stress).

In one embodiment of the device, the rail 388 (or other structure connecting the proximal and distal ends of the device) is made more compliant in tension than previously described. This allows for more relative motion of the tissue walls than does a rigid rail, while still serving the purposes of approximation and/or retention. Compliant rails may have other benefits, such as providing a more controlled and/or constant compressive force against the tissue than might a rigid rail, especially if the tissue between the proximal and distal wings increases (e.g., due to growth in pediatric patients) or decreases in thickness over time. Since the teeth of the rail are separated by a finite distance, a device that incorporates a toothed rail is not continuously adjustable in length between proximal and distal wings. In this case, compliance in the rail allows it to stretch to ‘in-between’ lengths otherwise unavailable. In lieu of or in addition to the rail being compliant, the wings or their mounting to the proximal and distal ends of the device may be compliant, to provide similar benefits. Compliant rails and/or other components may be fabricated from a material (preferably biocompatible) that is compliant (e.g., an elastomer) and assembled with other less compliant parts to form the final device. Alternatively, spring-like structures can be designed into a device made from relatively high-modulus material (e.g., metal) which provide the desired compliance. For example, the device can be designed such that a structure resembling an extension spring connects the distal end of the toothed rail to the distal block, instead of a direct connection as shown in the figures.

The device may be used to constrain the motion or location of tissue, or exert a force on tissue that is therapeutically beneficial. For example, a minimally-invasive procedure to treat heart failure may be achieved by using the device to create passive constraint of the left ventricle, in an analogous way to the CorCap cardiac support device of Acorn Cardiovascular (St. Paul, Minn.). In this application, one or more (typically more) relatively long devices are installed in the left ventricle such that the wings rest on the outside wall of the heart. The device spans from one surface of the ventricle to another (e.g., from posterior to anterior surface) and traverses the ventricle from within. Instead of the device being shortened enough to approximate these surfaces, it is shortened only enough to fully open the wings (if required) and to set the maximum size of the ventricle or the force that it is desired to exert upon it. In one embodiment of this application, several long devices are installed in the heart in minimally invasive fashion by piercing the heart with long but narrow-gauge needles, in different locations and/or orientations. In one embodiment of a device intended for treating heart failure, chains, cables, mesh, or other devices are attached to the proximal and/or distal ends of the device and lie on the exterior surface of the heart, to serve an additional constraining role on the heart.

Third Group of Embodiments—Edge-to-Edge Approximation with Winged Structures Located Opposite the Entry Side of the Approximated Tissue:

A device suitable for edge-to-edge approximation of tissue (i.e., tissue whose edges are separated laterally, and for which there is no substantial overlap), according to a third group of embodiments, exists and may be provided with expanding wings. Such a device may, however, have some significant differences as compared to the device of the first group of embodiments previously described. The goal is to replace sutures and staples in a variety of procedures with a device that can be delivered with one or more of the following benefits: (1) In a minimally-invasive manner, through a small (e.g., 15-17 gauge) cannula; (2) In seconds, allowing rapid intervention; (3) With greatly-reduced skill and training requirements; and/or (4) From only one side of the tissue (i.e., without use of an anvil as is generally the case with staplers.

Devices of this group of embodiments address one or more of several major challenges in suturing for minimally-invasive surgery:

(1) Skill and complexity. The device approximates and retains tissue much like a suture, but is deployed using only a few simple motions (in confined spaces, if necessary). A straight cannula is passed through the tissue walls and withdrawn, and no complex motions or knot tying is needed.

(2) Speed. Approximating tissue using suturing can easily take several minutes of stitch placement and knot tying. By comparison, a set of devices which accomplish the same purpose can be deployed in just tens of seconds; such faster procedures help reduce blood loss and morbidity.

(3) Complications. Unraveling of knots, fraying and breakage of suture due to handling, and other complications in suturing are eliminated by the device. The amount of tissue handling is less, thus tissue trauma can be reduced. If the device is made from (typically, biocompatible) metal, the stress relaxation causing loosening of polymer sutures is eliminated, and the device can be more thoroughly sterilized, reducing the risk of infection. Finally, the risk of suture pull-out under tension can be reduced, since multiple devices can be delivered to a surgical site, and then tightened a little at a time in alternation.

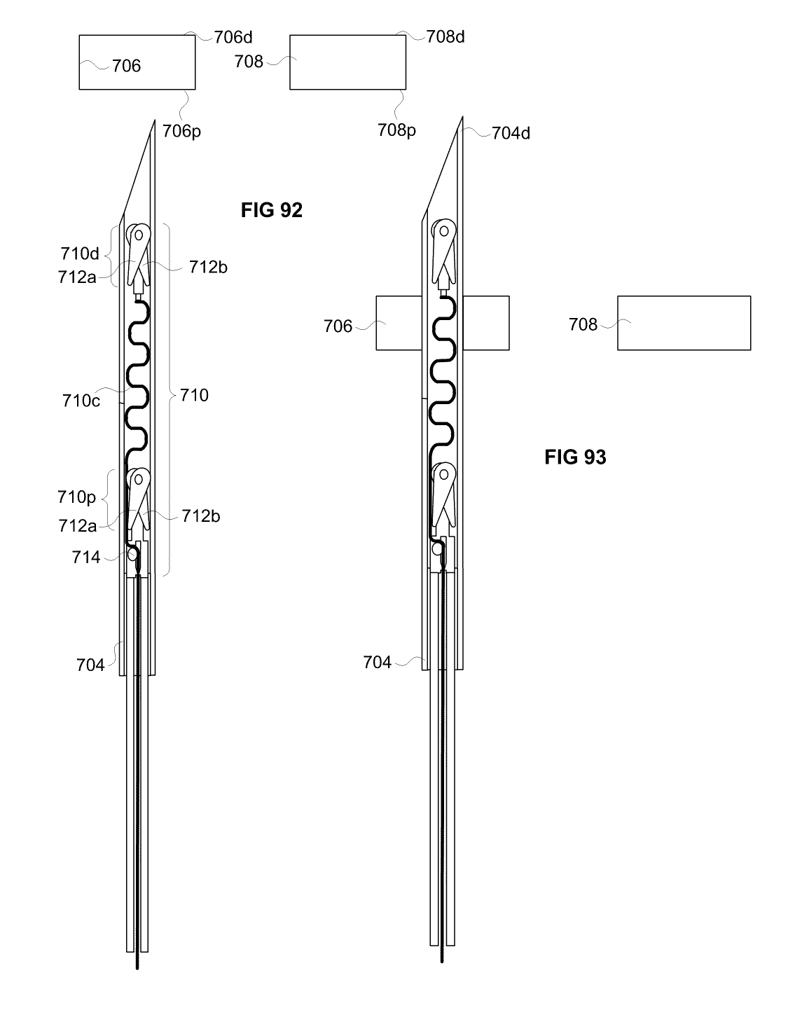

Devices of this group of embodiments can approximate tissue in a rapid manner, needing only simple motions. An example device and method is shown in FIGS. 92-100. These example devices function somewhat as ‘micro-harpoons’. The device and method depicted in these figures Is intended to be exemplary in nature and other embodiments may use other structures and functional relationships.

In FIG. 92, the two walls of tissue 706, having proximal and distal sides 706p and 706d, respectively, and 708, having proximal and distal sides 708p and 708d, respectively, to be approximated are seen. Also shown is a cross-sectional view of delivery needle 704 (e.g., 14-17 gauge, i.e. approximately 1.4 mm-2.1 mm in diameter), inside of which are components of the approximation device 710. Device 710 has two (proximal and distal), paired components 710d and 710p each with expanding (e.g., spring-loaded) wings 712a and 712b to provide anchoring in tissue. In this device, however, the pairs are oriented within the needle with the wing 712a and 712b located in the same orientation (i.e. oriented for distal opening), and are joined by a flexible chain 710c or other flexible element, at least some of whose links may be provided with teeth. The chain 710c (or other flexible element) passes over a pulley 714 attached to the proximal pair of wings (such that the force that approximates the tissue (i.e., tension applied to the chain) may be redirected along the needle axis (i.e., toward the surgeon). In this embodiment the needle incorporates a side slot 716 having bottom edge 718 which allows the chain 710C to pass through the slot when needed during delivery (e.g. the slot faces the location of the component 710d after delivery and the chain extends through the slot from component 710p to component 710d before delivery of component 710p, thereby allowing the needle to be inserted into tissue 708 without forcing the chain between the tissue and the needle. The needle 704 can be made of a variety of materials, such as standard stainless steel, or if desired, can be co-fabricated with the device. As with the devices of the first group of embodiments, the distance between proximal and distal wings can be shortened by pulling (i.e. the chain in this group of embodiments), and the distance will remain shortened by virtue of a ratcheting mechanism or catch 724 involving teeth and catches (other approaches can be used, such as the cleat-type mechanism of FIG. 73). FIG. 92 also shows push tube 722 and

In FIG. 93, the left-hand tissue wall 706 has been pierced by distal end 704d of needle 704.

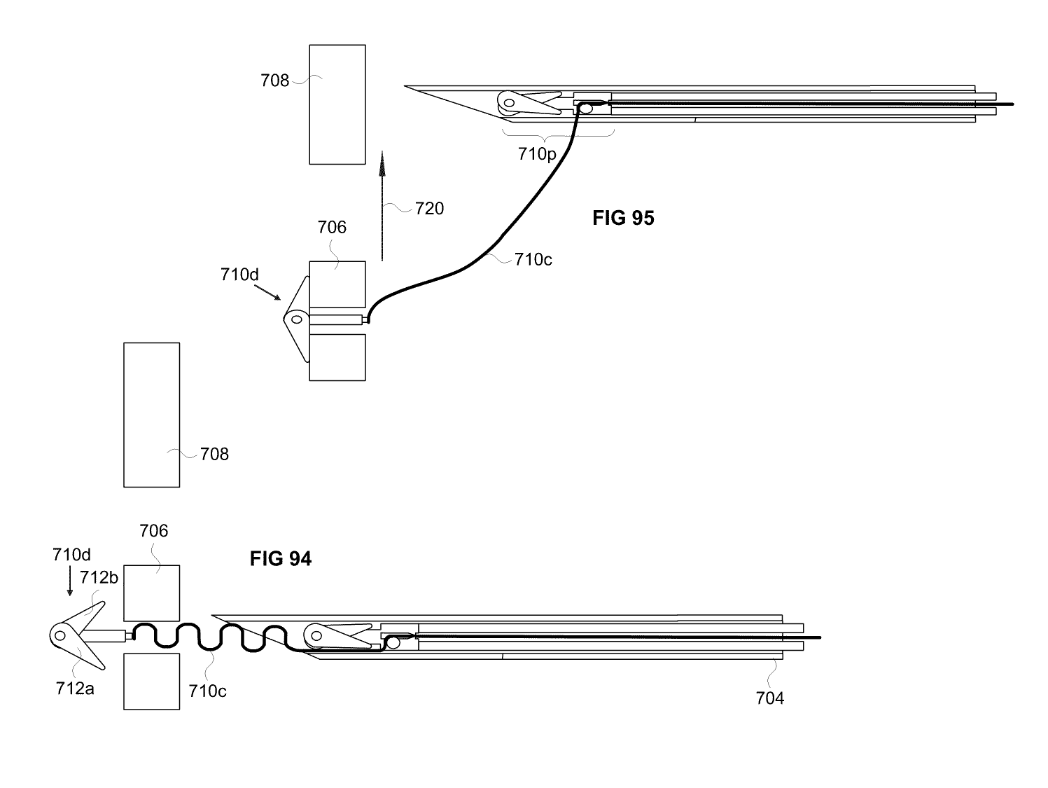

In FIG. 94, the needle 704 has been withdrawn while holding the push tube 718 in place, dragging along a chain 710c and also leaving behind distal end 710d, of component 710, with wings 712a and 712b which have expanded part way (i.e. rotated open) but have not yet been pulled back to engage the distal side 706d of wall 706 to anchor end (i.e. the distal end) of chain 710c. In some alternative embodiments, the needle is not necessarily moved in a direction shown by arrow 720 which is parallel to the surface of proximal side 706p of wall 706 or the proximal side 708p of wall 708. In these other embodiments, particularly if the needle is passing through a small port in a delivery catheter or other opening, it may instead by tilted in order to access the left-hand wall 706 and/or right-hand wall 708, using the port as a fulcrum, as is common in minimally invasive surgery. In general, devices of this embodiment may be delivered using only three degrees of freedom. Three degrees of freedom are sufficient to deliver the device; when a fulcrum is present, the degrees of freedom would be two rotations about the fulcrum and one translation axis (parallel to the needle axis). While folded inside the needle 704, chain 710c is quite rigid in compression along the needle axis (in practice the folds may be packed more tightly together than is shown), and thus serves as an extension of the push tube 718, preventing the distal wings from moving proximally while the needle is withdrawn. In other embodiments other elements may be used to provide the rigidity or force necessary to move the distal set of wings outward from the needle or to hold the set in place as the needle is extracted.

In FIG. 95, as the needle has moved to the right wall, the distal pair of wings has extended fully, and the chain has unfurled, passing through the slot 716.

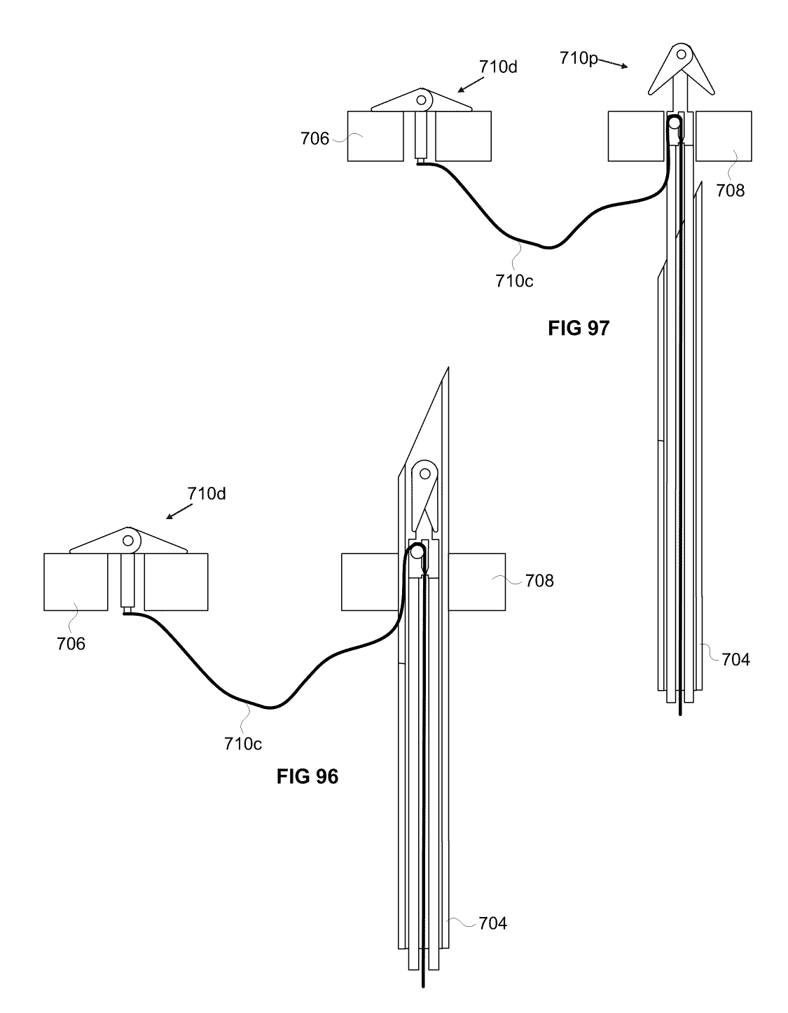

In FIG. 96 the right-hand wall 708 has been pierced by needle 704.

In FIG. 97, the needle 704 has been withdrawn, while the push tube 718 is held, leaving behind the proximal end 710p of device 710 in this embodiment) with wings 712a and 712b being partially opened but not yet seated against the distal end 708d of wall 708.

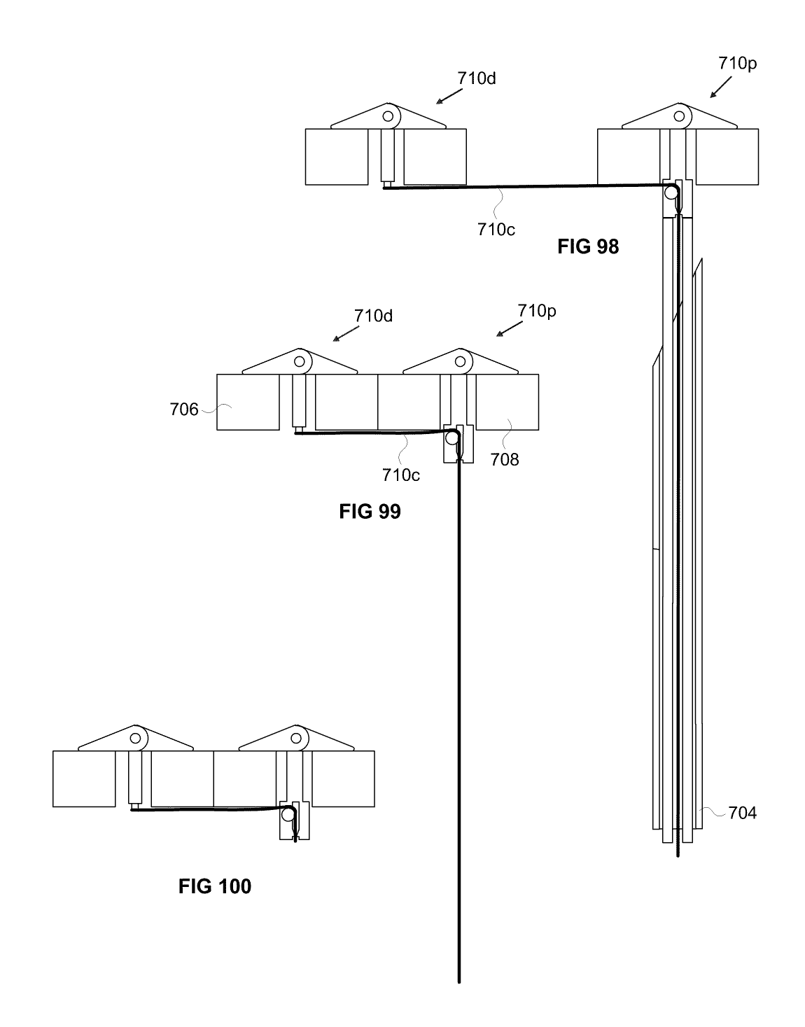

In FIG. 98, the chain has been pulled, tensioning it and forcing the proximal wings 712a and 712b of element 710p against the left-hand wall, fully extending them.

In FIG. 99, the needle 704 and push tube 718 have been withdrawn (if desired) and the end of the chain 710c has been pulled to appose the two tissue walls 706 and 708.

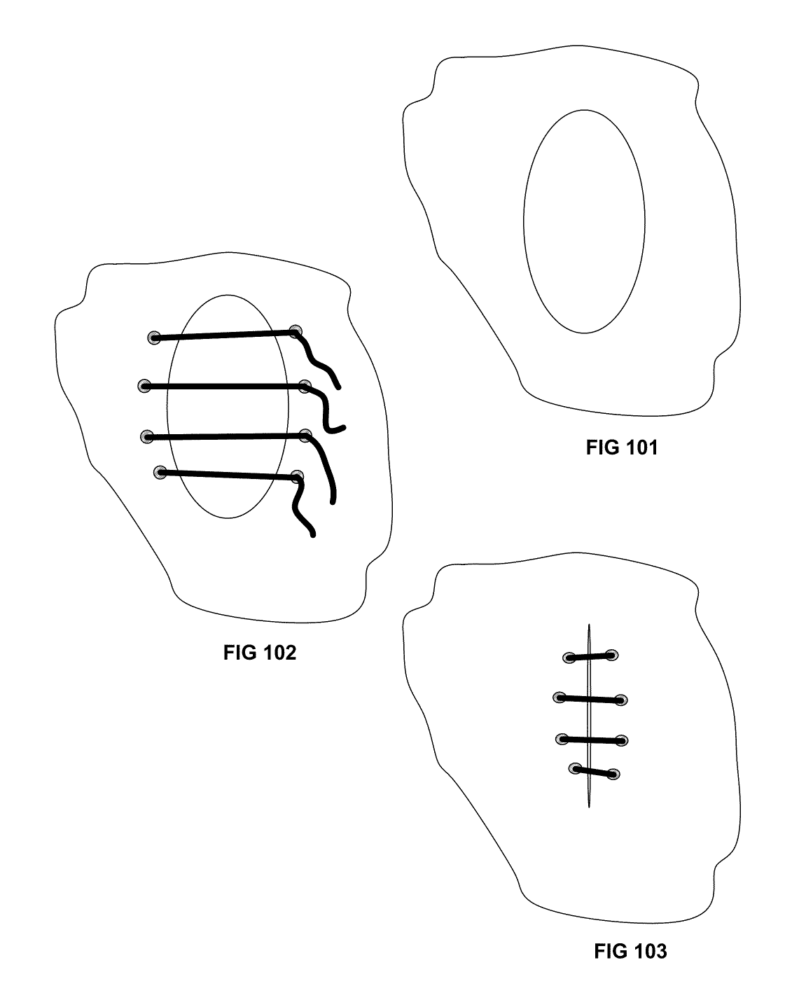

Finally, in FIG. 100, the excess portion of the chain has been removed. This can be done simply by cutting the excess chain using a tool such as wirecutters or scissors. Or, since it is composed of separate links (for maximum flexibility), the protruding links can be designed for removal. In some embodiments, this can be accomplished by allowing links to separate from one another when a link is rotated with respect to another adjacent link, yet preventing such rotation for all links other than the link at the location where it is desired to break the chain. The ideal location to break the chain is where the chain exits the right-hand side of the device, and this is where the chain has been broken in FIG. 100. As an example of a mechanism that allows rotation only when a link is in the proper position, a catch may be provided on each link to prevent rotation of an adjacent link with respect to the link; this catch can then be disabled so as to allow rotation (and thus breaking the chain), by contact with a structure that protrudes from the proximal end of right-hand side of the device. In alternative embodiments, the push tube may include an element at its distal end the engages the first chain link below the catch 724 (or some other chain link below that) to activate or enable a release mechanism that may allow disassembly of two adjacent links. Alternatively, the link held by the catch may be held in a fixed position while the push similarly engages a chain link that is at least one link separated from the link held by the catch, and upon rotation of the push tube, the intermediate link can be preferentially broken.

In lieu of immediately removing the excess chain as in FIG. 100, it may be preferred to first deliver multiple devices in an area, and then tighten each gradually in alternation much as one tightens shoelaces or a wheel on a car. Such gradual tightening can minimize the risk of pull-out, and allow larger apertures, and/or apertures in higher-modulus tissue, to be closed. An example of such a process is illustrated in FIGS. 101-103.

The exemplary devices shown in FIGS. 86-100 are intended for approximating tissue walls that are not excessively thick such that the wings can expand on the distal sides 706d and 708d of the walls. For thicker tissue, of if preferred, alternative anchors which do not need to fully penetrate the tissue can be used, such as those shown in FIGS. 67A-67E and FIG. 74. For endoscopic/laparoscopic surgery, the needle can be introduced through a port. Alternatively, for endoluminal/endovascular surgery, it can be attached to or guided by a long catheter, and for open surgery, it can be attached to a suitable short control handle.

As with the device of the first group of embodiments, the device may include springs or other elements in the chain or elsewhere to provide extra compliance in the device, as well as tension-limiting clutches to allow elongation.

Multiple devices may be delivered through a single needle, either by pre-loading these into the needle, or loading them during the surgical procedure from the proximal end of the needle. A magazine can also be provided, along the lines of the device of the first group of embodiments.

In some embodiments, multiple devices may be deployed simultaneously (e.g., to close an aperture that is too long to be closed by a single device) by ganging together the needles so that they penetrate the tissue in several locations at the same time, with each needle delivering a device.

As noted above, devices described herein may be used in multiples to approximate tissue, and optionally, a gradual tightening approach may be employed to reduce the pull-out stress on the tissue and/or allow a larger aperture to be closed. For example, atrial and ventricular septal defects of the heart are currently closed by sutures alone (in an open procedure) unless the aperture is too large and a sutured patch becomes necessary to span the aperture. FIG. 101 shows such an aperture in tissue. Using multiples of the device of the second group of embodiments (for example) as shown in FIG. 102, the aperture may be spanned initially and then (FIG. 103) closed by pulling on the chains. If this is performed in small increments moving from one chain to the next and repeating in the same sequence or reverse sequence, a larger atrial or ventricular septal defect, or other opening, could be closed without a patch.

Fourth Group of Embodiments—Edge-to-Edge Approximation Devices using Multipart Clips:

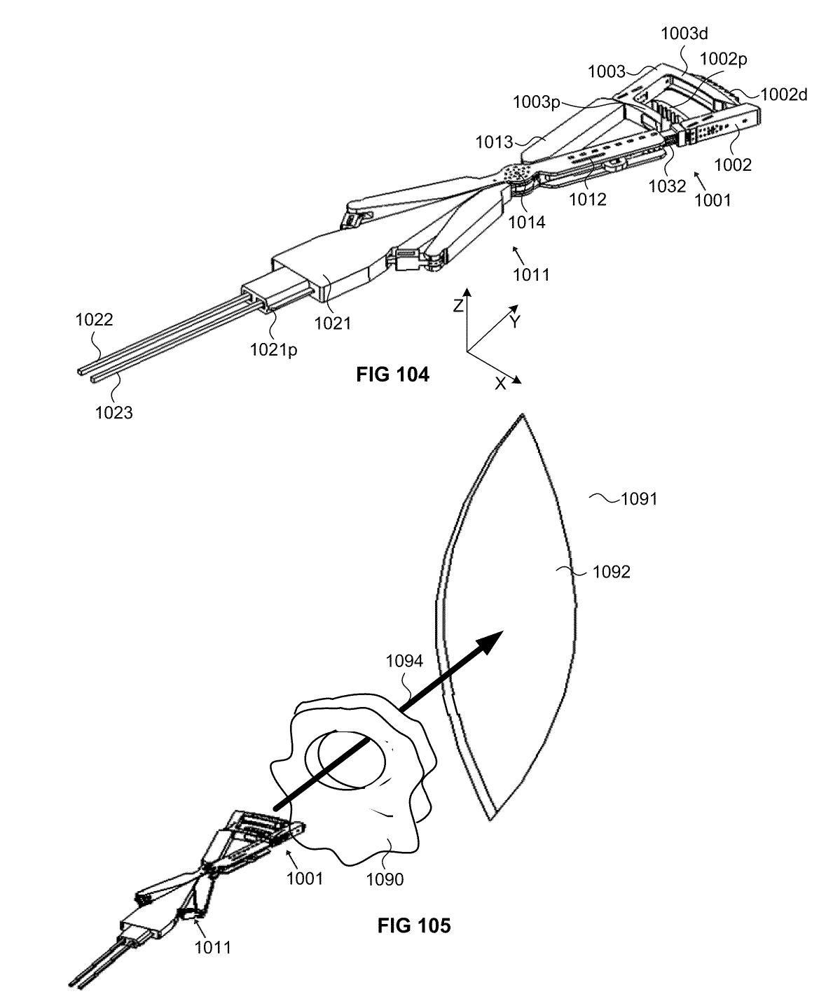

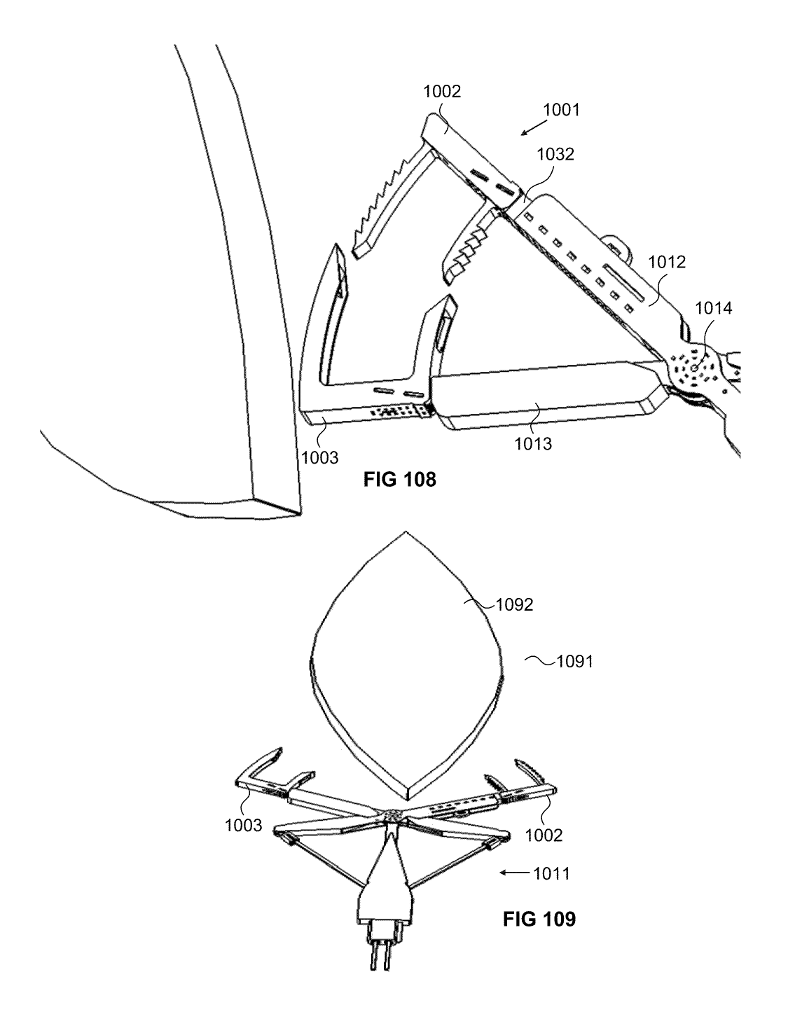

FIG. 108 illustrates an exemplary device and deliverable clip of an alternative group of embodiments that may be used for edge-to-edge tissue approximation which approximating tissue of a another group of embodiments which is suitable for edge-to-edge approximation of tissue is shown in FIG. 104. Again, the goal is to replace sutures and staples in a variety of procedures with a device that can be delivered while one or more of the following benefits is achieved:
In a minimally-invasive manner, through a small incision (e.g., 4-5 mm).
In seconds or less (once positioned), allowing rapid intervention, and the approximation of rapidly-moving structures such as heart valves.
With greatly-reduced skill and training requirements.
From only one side of the tissue.

As shown in FIG. 104, the device consists of both a clip and a clip applier. The same applier may be used to deliver multiple clips in succession, by reloading. The clips and applier may be fabricated together using the multilayer metal fabrication process previously described, or another process, or they may be fabricated separately, and be of different materials (e.g., the clips may be made of a biocompatible material such as nickel-titanium, whereas the applier may be made from a material such as nickel-cobalt). The clips are ‘split’, comprising two portions (i.e. two “halves”) which interlock with one another. Each half-clip typically includes two prongs, one distal and one proximal but additional prongs may be provided in some alternative embodiments. In use, the distal prongs of each half-clip penetrate the tissue walls to be approximated, at some distance from the edges, much like needles used for suturing. The applier holds the half-clips, causing them to pierce the tissue, and then brings them together in alignment to form an interlocking unit while approximating the tissue, analogous to an interrupted stitch.

In FIG. 104, the two arms of the applier may be seen; these articulate about a pivot, somewhat like scissors or pliers. At the proximal end of the applier are wires which are guided through the shaft of the applier and are connected to the proximal end of the arms to actuate them. In the embodiment shown, the wires may be independently manipulated to independently control the motion of the right and left applier arms. This may provide additional maneuverability and improve access to certain target apertures. In other embodiments, the wires may be joined together (e.g., proximal to the shaft) or replaced by a single wire connected to rigid, pivoting linkages which interface with the arms (as with laparoscopic forceps); in this case pulling one wire actuates both arms symmetrically. The proximal end of the shaft may be inserted into a length of tubing (e.g., a catheter) to lengthen it, with the wires passing through the tube lumen. In some embodiments, the wires may supply a pushing force (via their stiffness) to cause the arms to open as well as a pulling force to cause the distal portion of the arms to close. In some alternative embodiments, the arms may be biased in closed or partially closed position when in the applier and may be forced open by a compressed spring on the distal side of the pivot and/or a tensioned spring on the proximal side of the pivot so that only a pulling force or tensional force is required (i.e. to close the arms).

In FIG. 105, the clip applier and clips are seen in relation to two regions of tissue. The more distal tissue has an aperture to be closed (the ‘target aperture’) by approximating the edges. An example of such tissue is an organ such as the stomach after making an incision (as may be done in the course of endoluminal/natural orifice transluminal endoscopic surgery) which needs to be closed at least partially to facilitate healing; such closure may be carried out endoscopically using a device of this third group of embodiments. Another example might be the leaflets of the mitral valve of the heart, which if joined in an edge-to-edge manner can reduce regurgitation; such surgery may be performed percutaneously using the device described herein.

In many cases, though not all, the target aperture is within the body, and the device must first enter the body through an access port, which is typically made as small as possible. FIG. 105 shows such a port in other tissue; just a small portion of the tissue is shown for clarity. The port is shown as circular (as may be the case, for example, if a trocar or introducer is used). However, since the cross-section of the device perpendicular to its longitudinal (proximal-distal) axis is much shorter along one axis than another, the port may be of the form of a linear incision parallel to the longer axis of the device. An introducer or trocar with an oblong or rectangular shape might also be used. Once inserted, the device can be rotated around its longitudinal axis if necessary. As shown in FIGS. 104-105, the clip applier is in the configuration that provides the smallest cross-section: with the arms at a small angle with respect to the shaft, and with the right half-clip extended distally on a slide, such that its prongs do not intersect those of the left half-clip. Without the slide, the device could not be as narrow (e.g., allowing the port do be as small as possible) since overlap of the prongs would need to be avoided to prevent them from interlocking prematurely. If making the device as narrow as possible is not required, or a means of preventing interlocking of the prongs (e.g., holding the catch heads away from the teeth of the right half-clip prong), means for controllably releasing interlocked prongs is provided, then the slide may be eliminated. In the device illustrated, the height of the device in the configuration shown is 0.75 mm and the width approximately 5 mm. The arrow in FIG. 105 shows the delivery path of the clip from outside the body, through the port, and to the target aperture. If the device is attached to a suitable device (e.g., a catheter), this path may in fact be non-linear and even tortuous.

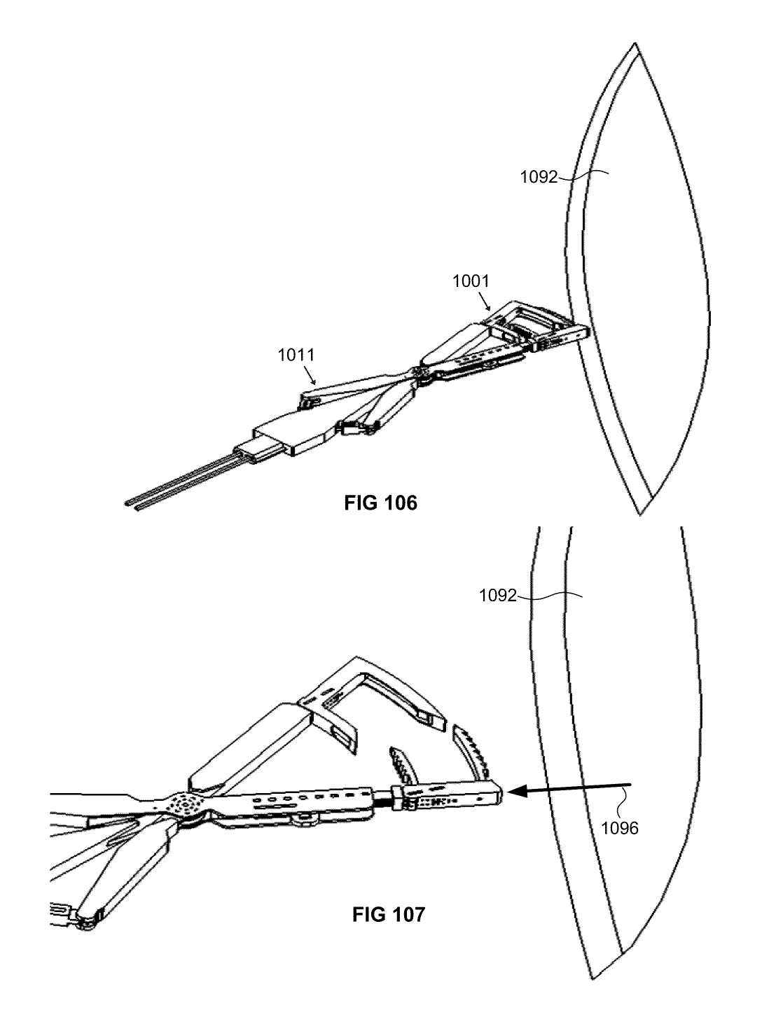

FIGS. 106-121 illustrate the steps involved in delivering the clips; for clarity, the port and the tissue surrounding it are no longer shown. In FIG. 106, the clip applier and clip has approached the target aperture. In practice, the clip applier and clip may be rotated about the insertion line to obtain a proper positioning relative to the aperture. In FIG. 107, the arms have been rotated outwards (i.e. the opens opened) enough to allow the right half-clip to be moved proximally on the slide, after which the two half-clips will interlock if brought together. The arms may be moved by pushing on the wires (which are designed to be stiff enough not to buckle under these conditions), dragging the tips of the distal prongs against tissue, etc. In some embodiments, as noted above, a spring may be provided to spread the arms, e.g. when tension is released from the wires. In FIG. 108, the slide has moved the right or top half-clip proximally where it locks into position, ‘arming’ the device so it is ready for use. Proximal movement of the slide can be accomplished by simply pushing the distal end of the right half-clip against tissue. In some embodiments, wires, chains, cables, or the like attached to the slide (or the right half-clip) that are either routed through a channel in the applier arm or external, can be used to pull the slide proximally. In some embodiments, the left vs. the right half-clip is held on a slide, and in some embodiments the half-clip is initially more proximal than it must be to interlock with the other half-clip, and must be pulled or pushed distally, instead of proximally in the embodiment shown. Such a motion might be provided by initially hooking the prong(s) of the half-clip on the tissue and pulling away the clip applier; however, the relative force required to move the slide must be less than that required to separate the half-clip from its holder, to prevent this from occurring prematurely.

In FIG. 109, the arms have been rotated to an angle sufficient to span the target aperture with some excess. In practice, it is desirable to penetrate the tissue with the prongs of the clip halves at points reasonably distant from the edges of the aperture. This desire must be, however, balanced with the limited width of the clip once delivered, and thus its limited capacity to accept tissue, as well as with additional force that may be required to deliver the clip if the points of penetration are too far from the edges. In the design illustrated, when the arms are opened to their maximum angle, the tips of the distal prongs on the half-clips are about 12.5 mm apart, allowing the device to be used to close apertures up to about 10 mm in width at the widest point. In some implementations, it may be possible to close wider apertures with this design by using initially applied clips to close narrower regions which pull wider regions close enough together to fall within the working region of this design. In still other embodiments, appliers with longer distal arm portions may be used to allow wider opening and thus enabling use with wider gaps. In still other embodiments, shorter arm sections may be better suited to approximate smaller apertures because the penetration angle of the distal prongs can be steeper). In multiple clip embodiments, once the tissue edges are approximated by the initial clip or clips, the distance between the most widely-separated edges will normally decrease (by a zipper-like action), allowing clips to be inserted there.

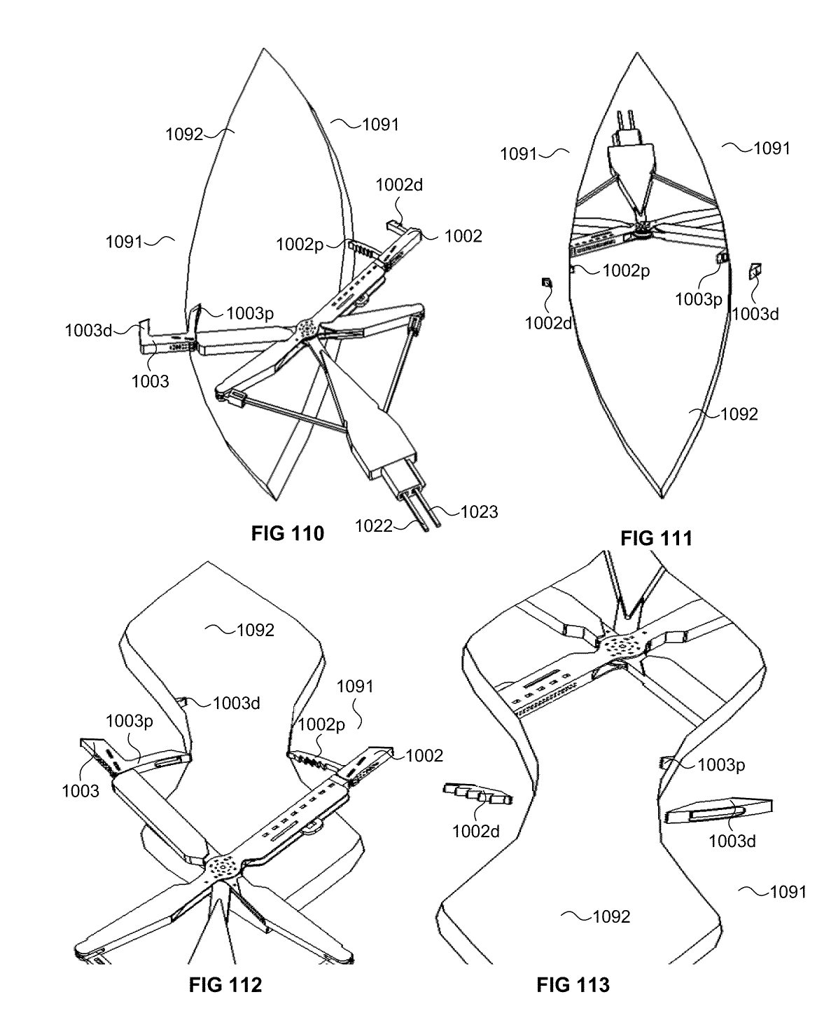

In FIG. 110, the clip applier and clip has been advanced toward the target aperture and the tips of the distal prongs have penetrated through the tissue, as can be seen in the view from the distal side of the tissue of FIG. 111. As shown, the tissue is thin enough that the tips can fully penetrate it and exit through the distal walls. However, thicker tissue can also be approximated using the device, in which case the prong tips may instead exit through the walls forming the edges of the target aperture. Clips with different distances between proximal and distal prongs can be designed, as may be useful to accommodate different tissue thicknesses. In FIG. 111, the wires have been pulled, rotating the arms to a smaller angle and bringing together the ‘skewered’ tissue edges partway. As shown, the tissue is reasonably elastic such that edge movement is somewhat a local effect, with the edges moving primarily in the region of the prongs. On the other hand, the edges of more fibrous tissue distant from the prongs may move a larger distance than is shown. FIGS. 113-114 are views from the distal side of the tissue at this stage of delivery.

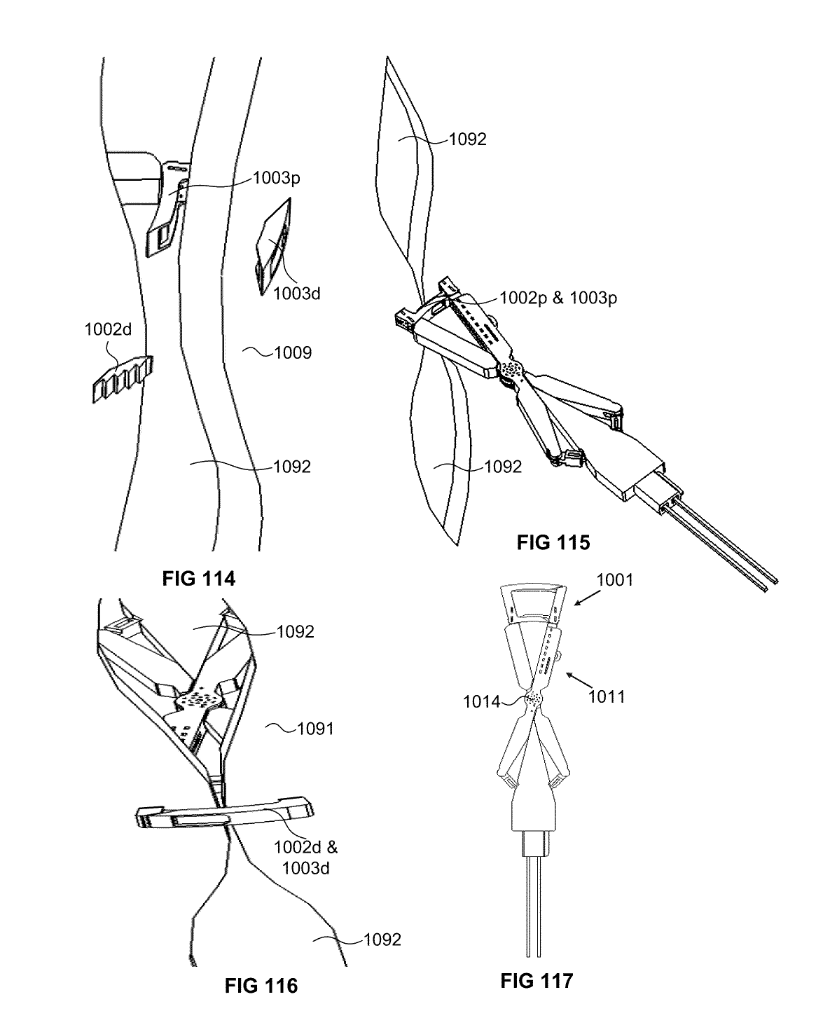

In FIG. 115, the wires have been pulled further, bringing the arms to their smallest angle with respect to the shaft; this delivers a clip of minimum width. The edges of the tissue have been brought together still further, and in the illustrations, now are nearly in contact. FIG. 116 is a view from the distal side of the tissue of the clip. As the arms move inwards, the two half-clips ratchet together in a unidirectional fashion. The teeth of the right half-clip prongs become engaged by the catch heads of the left half-clip prongs, interlocking the two half-clips together to form a single unit—a clip that is a continuous, closed loop. Since there are multiple teeth on the prongs, the width of the clip as-delivered can be varied by adjusting the final position of the arms. Of course, the minimum and maximum widths available to the clip are a function of its design (e.g., the length of the prongs). The width can thus be adjusted to accommodate different amounts of tissue captured by the clip. Also, the width can be adjusted so that the edges of the tissue do not fully come into contact, or at the other extreme, are squeezed together. FIG. 117 is a top view of the clip applier and clip with the clip at minimum width; the tissue is not shown for clarity. If desired, the clip may be delivered such that it is wider than its minimum width (e.g., at maximum width). It may then be reduced in width by use of conventional instruments such as forceps or tweezers, or by re-engaging the clip applier. Such ‘post-tightening’ of the clip would allow, for example, gradual tightening of a number of clips in ‘shoelace’ fashion as described above for the device of the second group of embodiments.

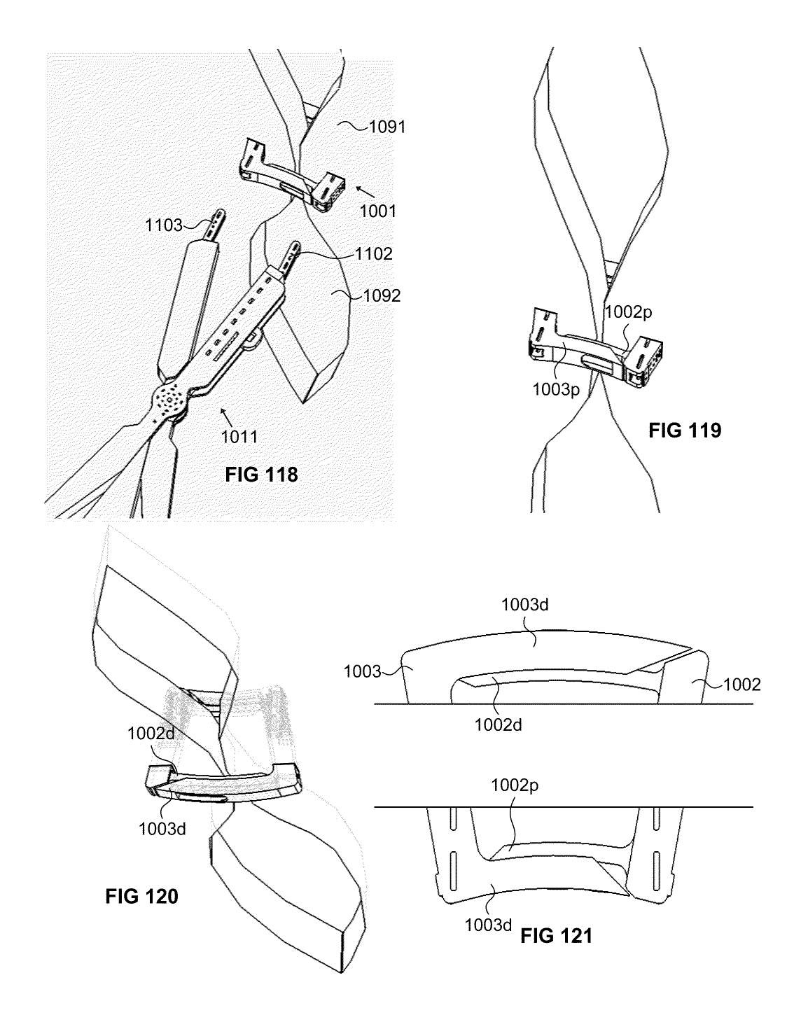

In FIG. 118, the clip applier has been pulled in the proximal direction (i.e. along the line of entry toward directly or indirectly toward the entry point), causing it to separate from the clip. Visible here are the left and right half-clip holders, which fit into channels in the half-clips. The clip is now fully delivered and the clip applier can be removed through the port. FIGS. 119-121 are views of the clip as delivered in the tissue. FIG. 119 is a view from the proximal side of the tissue; FIG. 120 is a phantom view from the distal side of the tissue, and FIG. 121 is a top view (assuming the plane of the tissue extends along a vertical plane. Additional clips may be delivered to the target aperture if required to approximate the tissue edges.

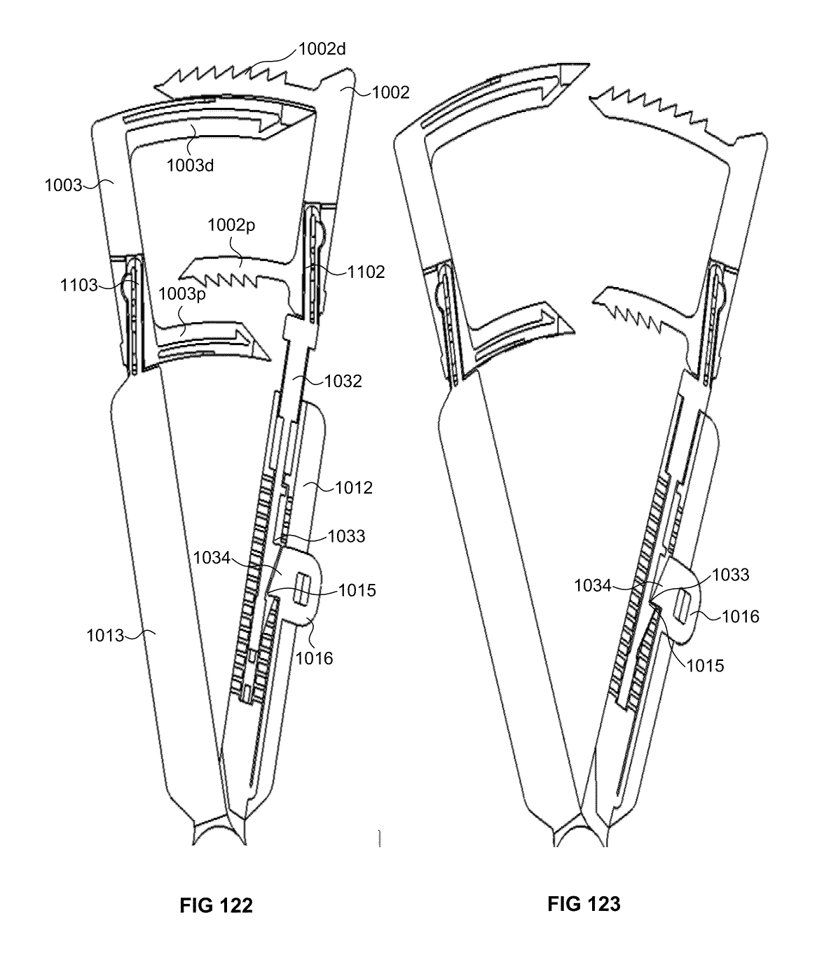

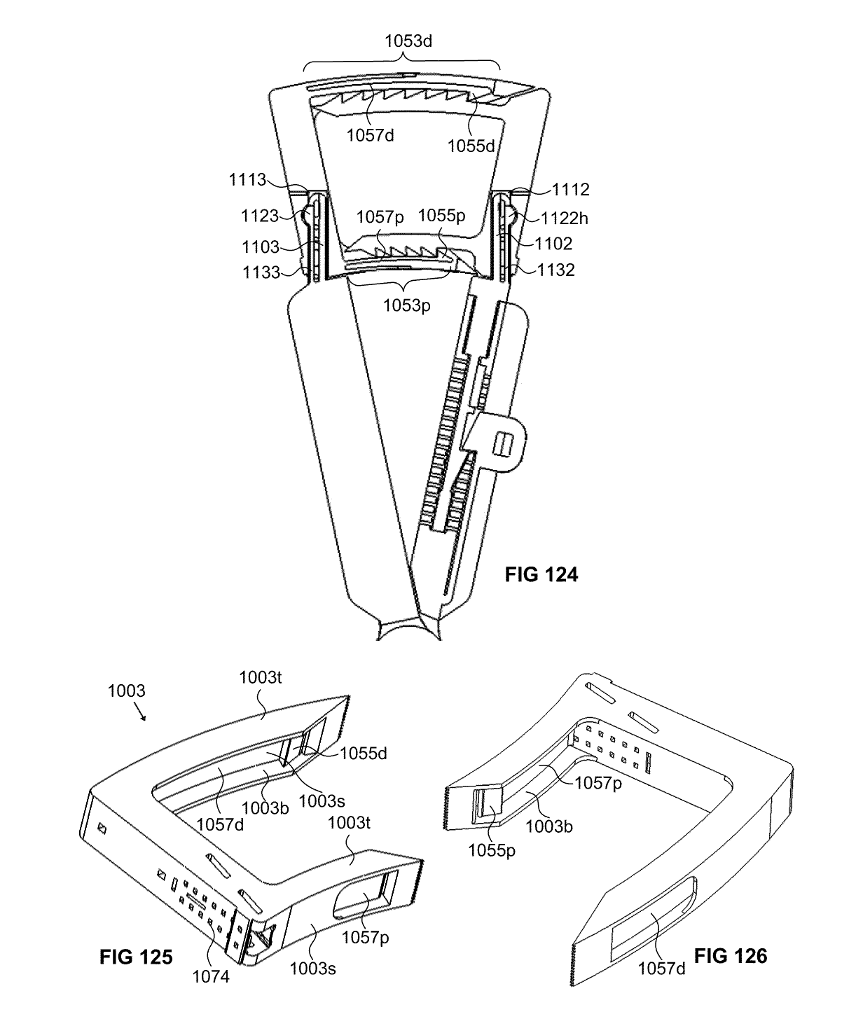

FIG. 122 is a top view of the half-clips and the two distal arm sections of the clip applier. The arms and slide are in the same position as in FIGS. 104-106; the arms are at a small angle with respect to the shaft, and the right half-clip is extended distally on the slide. FIG. 123 is a similar view in which the arms and slide are in the same position as in FIGS. 107-108; here the arms have been rotated outwards partway, and the right half-clip has been moved proximally on the slide. FIG. 124 is a similar view in which the arms and slide are in the same position as in FIGS. 115-117. Here the half-clips have interlocked together and the channels for the half-clip holders are roughly parallel to each other and to the clip holders. The clip applier may therefore be easily pulled away from the delivered clip (which is retained on the applier primarily by the clip retention catch heads). If the half-clips need to be removed from the applier when the arms are at other angles, this can be accommodated by making the channels in the half-clips wider to allow some rotation of the half-clips on the holders. Alternatively, pivots or flexures may be provided (e.g., for the holders) to allow some rotation of the holders. If the intent is to deliver the clip at a greater width than its minimum width (e.g., because of a desire to tighten it later using another tool), then the angle of the channels and holders can be modified so that the holders are substantially parallel (so the clip can be easily slid off the holders) at the angular separation of the arms corresponding to the desired clip width.

It will be noted in FIG. 124 that since the teeth of the right half-clip face away from each other (and the U-shaped prongs of the left half-clip are open toward each other), then the two half-clips, once engaged, are unable to slide in their own plane relative to one another such that they become separated (i.e., with the catch heads no longer engaging the teeth). In an alternative embodiment, the teeth of the right half-clip may face each other and the open sides of the left half-clip may face away from each other. In some embodiments, the teeth of both prongs may face in the same direction (e.g., distally), and another means is provided to prevent the two half-clips from inadvertently disengaging.

As may be noticed in FIG. 124, the teeth of the right applier arm have a slightly undercut angle (e.g., 5 degrees) which matches the mating surface of the catch head. This angle helps keep the catch head from becoming disengaged from the teeth when lateral tension is applied to the clip, effectively increasing the maximum rating of the clip.

The left half-clip is shown in FIGS. 125-128. As shown in FIGS. 125-127, the half-clip comprises a body and two prongs, one proximal and one distal. The distal prong normally penetrates tissue, while the proximal prong may not penetrate tissue, though it may in some circumstances. Since the arms rotate about a pivot when bringing the half-clips together, the prongs are not straight, but are arced; both prongs have a common center. Each prong is substantially U-shaped in cross section, with three sides: a bottom, a top, and either a distal (for the distal prong) or a proximal (for the proximal prong) side. Within each prong is a catch comprising a catch head and a catch beam. Additional, redundant catch heads can be provided to increase reliability of the clip if required. The catch head engages the teeth of the right half-clip, and the compliant catch beam bends to allow the catch head to ride over the teeth as the two half-clips are brought together. The top and bottom sides minimize contact with the catches when the prongs penetrate tissue, since the catches are recessed beyond the edges of the top and bottom sides. Moreover, the top and bottom sides serve as stops to prevent the right half-clip from moving vertically (i.e., parallel to the clip applier pivot axis) which may cause it to disengage from the left half-clip.

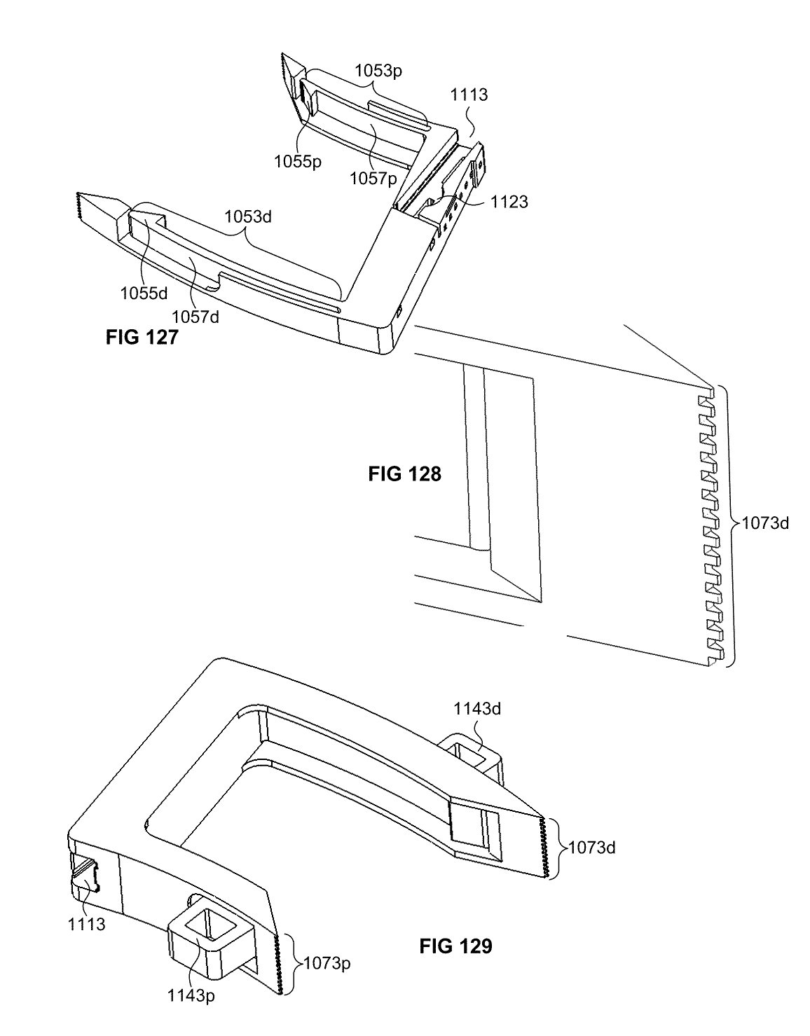

Another key feature of the half-clip is a channel in the body that accommodates the left half-clip holder (a similar channel is provided in the right half-clip for its holder). Also visible are release holes which are provided only when the left half-clip is fabricated using the multi-layer fabrication process previously described and when the channel already occupied by the left half-clip holder, such that the left half-clip is pre-assembled onto the clip holder. If the left half-clip is fabricated separately by whatever method, and then placed on the left half-clip holder, these holes may be eliminated. FIG. 127 is a sectional view of the left half-clip, and FIG. 128 is a detail view of the tip of one of the prongs, showing the crenellated edge of some embodiments, which may increase the tissue penetration capability of the prong. Windows in the proximal and distal sides facilitate release of a sacrificial material that may be used during formation of the device and in the case of the distal prong, allows the half-clips to be fabricated in the configuration similar to that in FIG. 122 (though with the arms more widely spread) without the prongs being too close to one another. In some embodiments, the windows also give access to lugs, which may be provided (FIG. 129) on each catch arm to allow the two half-clips to be separated after they have interlocked. The lug may include a hole as shown to facilitate pulling on it, e.g., using a hook.

When the distal lug is pulled distally and the proximal lug is pulled proximally, the catch heads no longer engage the teeth, the right half-clip can be separated from the left half-clip, and the clip thus removed from the tissue.

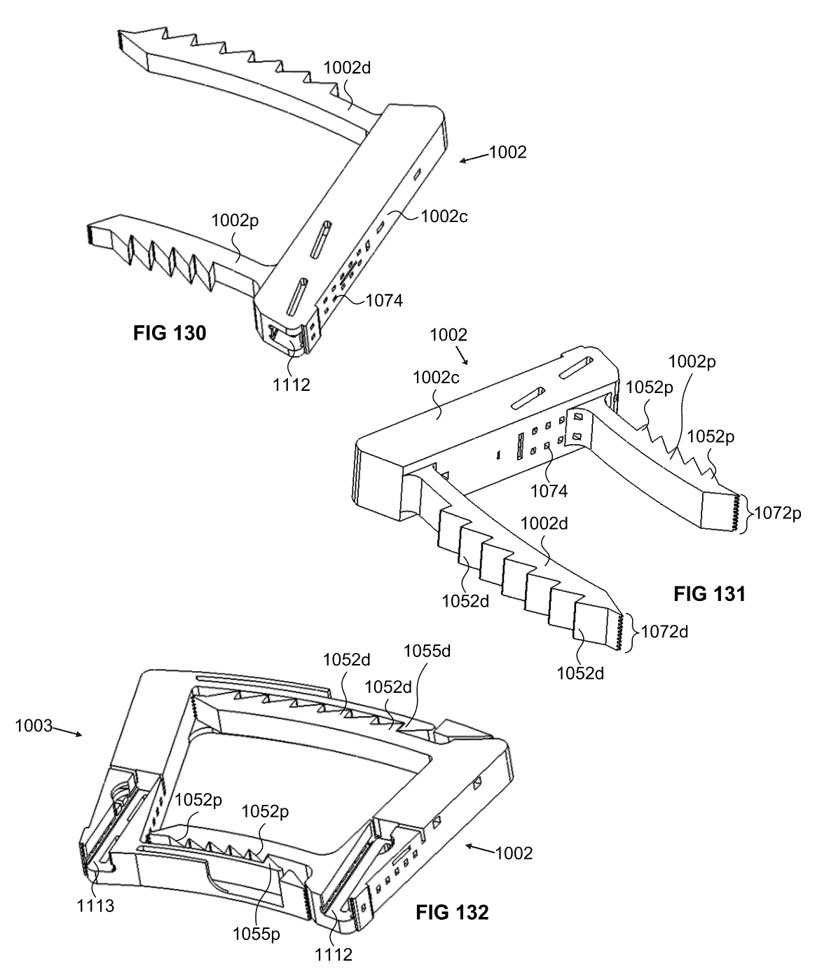

The right half-clip is shown in FIGS. 130-131. It too comprises a body with proximal and distal prongs; however, the shapes of the prongs are different from those of the left half-clip, and complement them. Each prong comprises at least one tooth (more than one allows the width of the clip to be adjusted) designed to engage the catch head on the left half-clip. The prongs are arced like those of the left half-clip, and share the same center of curvature. To allow the teeth of both proximal and distal prongs to ratchet into the left half-clip one at a time, the pitch of the teeth of the distal prong must be spaced at a greater linear pitch (but the same angular pitch) than that of the proximal prong teeth.

The prongs are of a height (measured vertically) that allows them to fit between the top and bottom sides of the left half-clip prongs. Similar to the left half-clip, a channel is provided in the body to accommodate the right half-clip holder and release holes are provided if needed. Like the left half-clip, the edges may be crenellated in some embodiments.

FIGS. 132-133 are sectional views of the assembled clip, with both half-clips fully engaged (i.e., the clip width is minimum). As may be seen the proximal prong catch beam is far enough from the proximal side of the left half-clip (and similarly, the distal prong catch beam is far enough from the distal side of the left half-clip) that the beams can deflect when pushed by the teeth of the right half-clip.

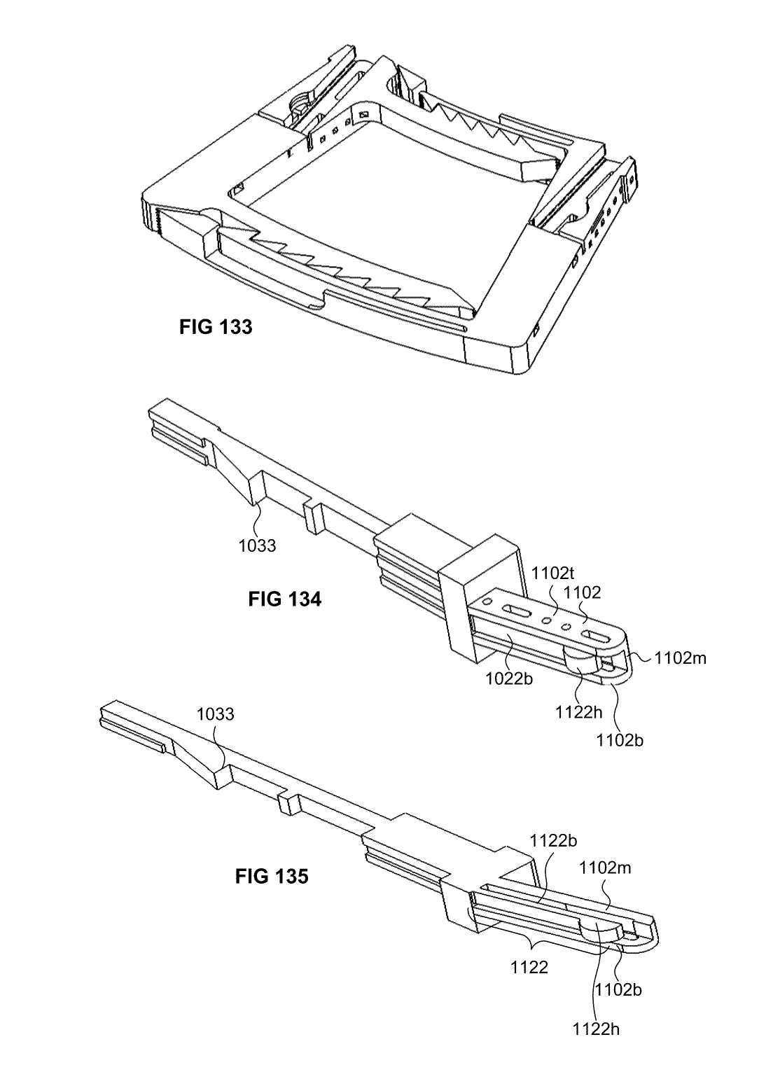

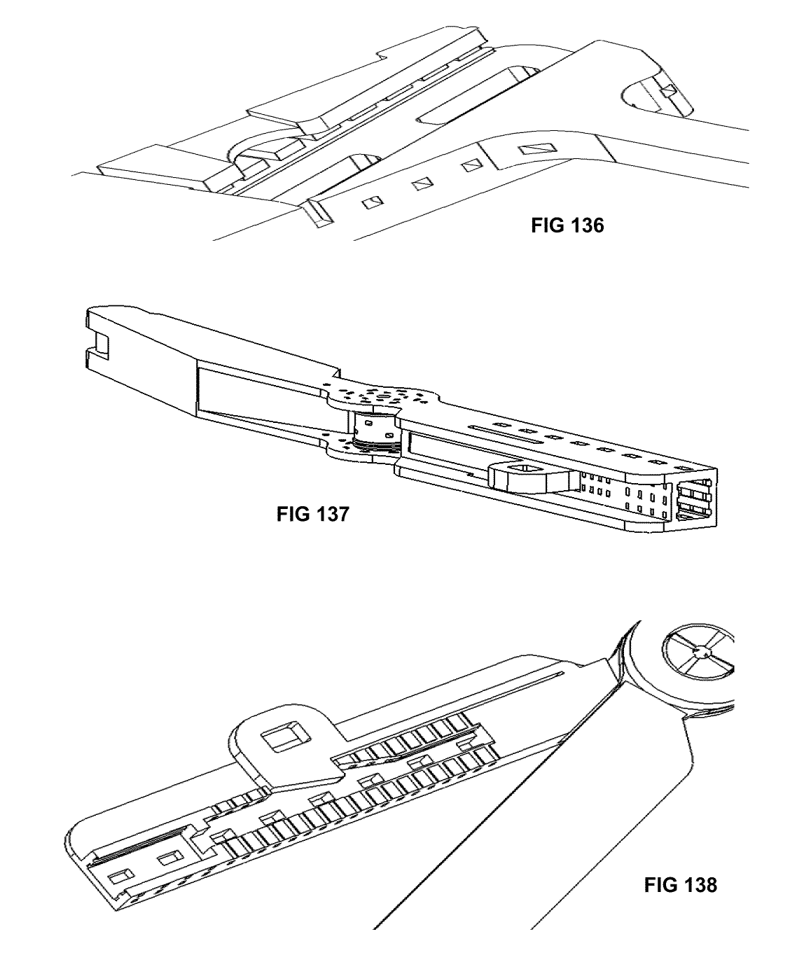

FIGS. 134-135 (the latter, a sectional view) depict the slide in isolation from the rest of the clip applier. At the distal end of the slide is the right half-clip holder. The holder is substantially U-shaped in cross section, with three sides: a bottom, a top, and a medial side. Within the holder is a catch comprising a catch head and a catch beam. The catch head is preferably rounded (vs. sawtooth-shaped) so it functions bidirectionally, allowing the right half-clip to be released from the holder and replacement right half-clips to be placed on the holder. FIG. 136 depicts the right half-clip in cross-section, showing the recess within the channel for the catch head.

Proximal to the holder is a base which rests against the distal end of the right applier arm when the slide is moved to its fully distal location. Proximal to the base is the distal block; along with the proximal block shown in the figure, this comprises the ‘bearing’ portion of the slide: both blocks slide in channels within the distal arm section of the right applier arm. Some embodiments of the slide may co-fabricate it using the multilayer process previously described, along with the rest of the clip applier in a pre-assembled configuration. In these cases, to reduce the horizontal (i.e., within-layer) clearances (e.g., 20-30 microns) between slide and applier arm channel (necessitated by the minimum feature size of the fabrication process), projections may be added both to the slide (FIG. 134) and also to the channel (FIG. 137) of the right applier arm in which the slide translates. Since the projections on the slide are not on the same layers as those on the arm, the minimum feature size design rule is not violated. Projections may also been used on the sidewalls of the holder and the channel within the right half-clip for the same purpose of reducing clearance if the right half-clip is co-fabricated along with the holder in a pre-assembled fashion.

In some embodiments, the slide also includes a slide stop which, by coming into contact with a slide stop rest on the inside of the right applier arm, prevents the slide from falling out. In the figures, the stop is shown proximal to the distal block. Proximal to the stop in the figures in some embodiments is a tooth which can be caught by the slide catch head of the right applier arm (FIG. 142); this retains the slide when it is pushed (or pulled) distally as in FIG. 107.

The right applier arm is shown in FIGS. 137-138 (the latter a sectional view). The arm comprises a proximal arm section (on the left side of the pivot) and a distal arm section (on the right side of the pivot). Within the distal section is a channel that accommodates the slide of FIG. 134. At the distal end, projections may be seen that articulate with the distal block of the slide as already described. Partway down the distal arm section is the slide catch head, which captures the slide tooth when the slide is moved fully proximally. The head is attached to a beam and is provided (if reloading/reuse of the clip applier is intended) with a lug that allows the catch head to be pulled laterally to release the rail so it can move to its distal position. Near the center of the applier arm is a pivot pin which articulates with both the left applier arm and a pivot shaft. Proximal to the pin is the proximal arm section, to the proximal end of which is connected the wire that moves the arm. The right applier arm (like the left applier arm) is made as thick as possible in the horizontal plane to minimize bending when approximating stiff tissue. The arms are also designed to be reasonably high/thick (e.g., 0.75 mm or greater) to avoid twisting and other distortions.

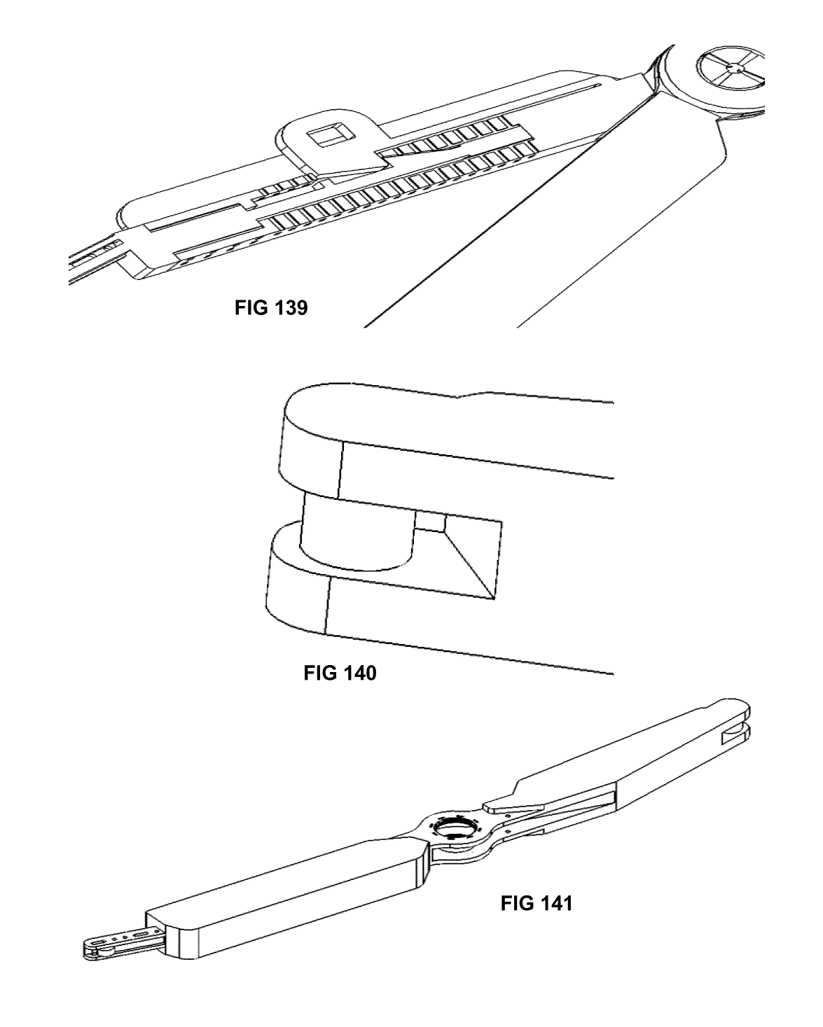

FIG. 139 is similar to FIG. 138, but with the slide in place at its most proximal position. It can be seen how the catch head engages the slide tooth to capture it proximally. It may also be seen how the slide stop rest can come into contact with the slide stop, preventing the slide from falling out of the right applier arm. At the proximal end of the right applier arm is a groove within which is a pin that articulates with the wire connector; this can best be seen in FIG. 140.

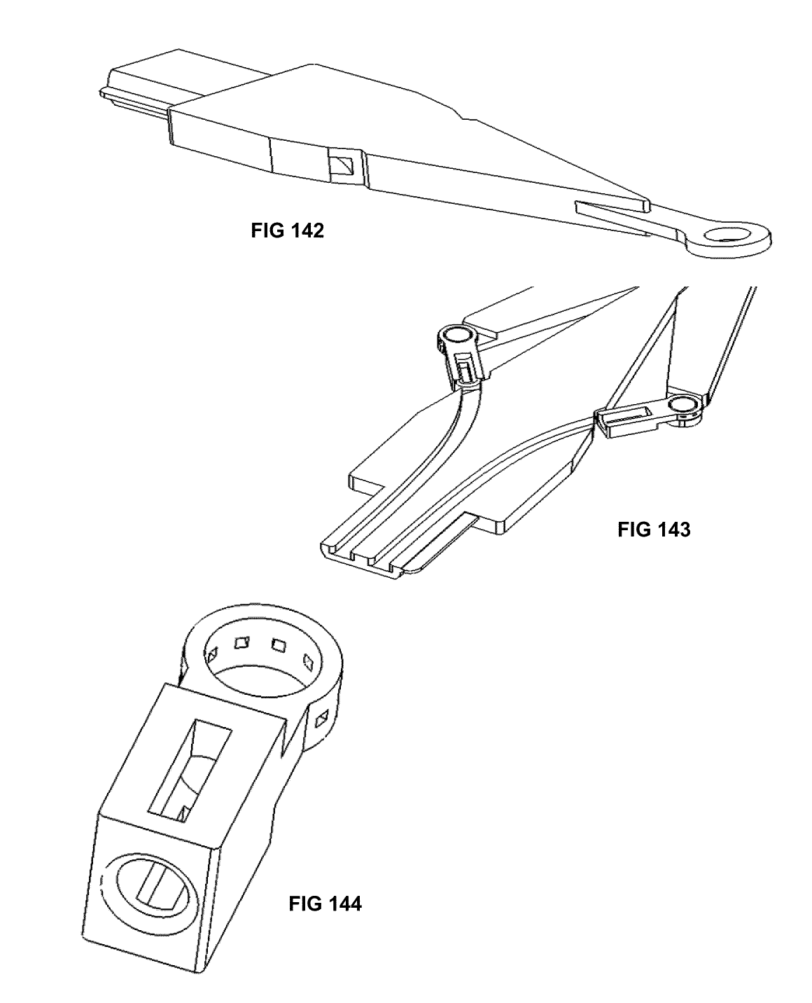

The left applier arm is shown in FIG. 141. Its design is similar to and in some ways a mirror image of the right applier arm, but with a few differences. Rather than having a channel to accept a slide (no such slide is needed for the left half-clip), the left half-clip holder (the mirror image of the right half-clip holder) is integral to the arm. Secondly, rather than a pivot pin near the center, there is a pivot hole that articulates with the pin in the right applier arm. The proximal end of the left applier arm includes a groove and pin for the wire connector; this is the mirror image of the right arm.

FIG. 142 shows the shaft in isolation. The shaft has a support for the applier arms at its distal end. Approximately in the middle of the shaft are the distal apertures for the wires that move the applier arms. At the proximal end of the shaft is the tongue, which may in part have a cross section that forms a portion of a circle (as shown) which can be inserted into the end of a length of tubing (e.g., a catheter). To prevent rotation of the shaft with respect to the tubing, a key is provided on a portion of the circular cross-section to fit into a slot in the tubing.

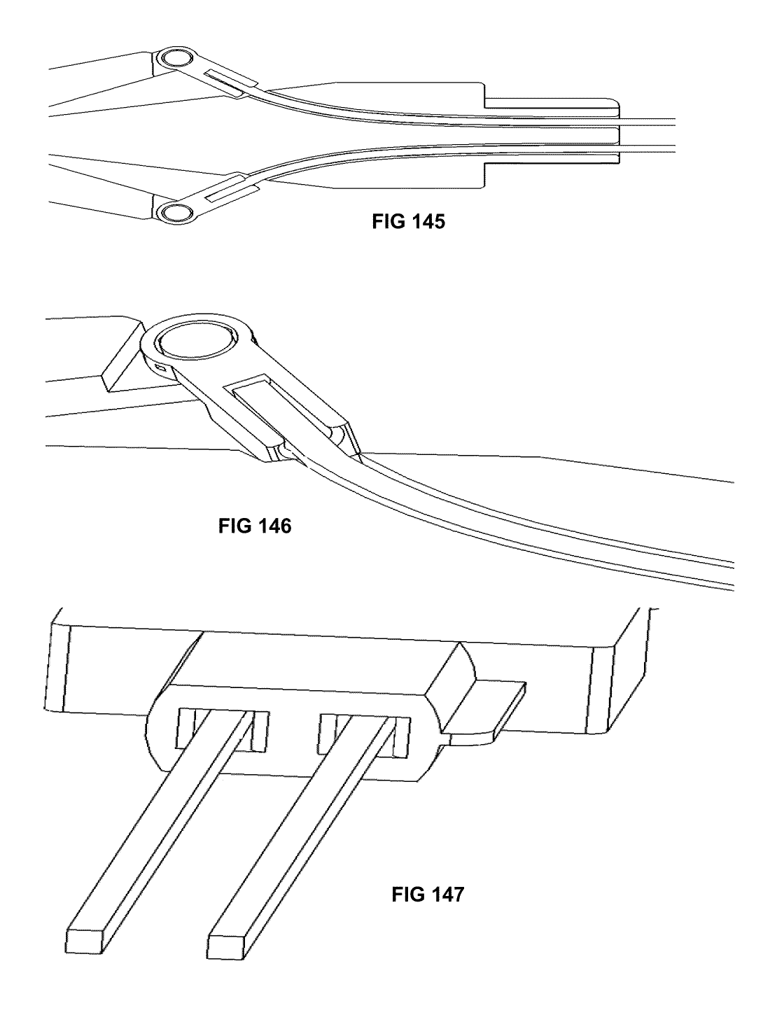

FIG. 143 is a sectional view of the proximal end of the shaft along with portions of the right and left applier arms and wire connectors; the wires are not shown for clarity. Gradually-curving wire channels are provided to guide the wires between the proximal and distal apertures. In this way the direction of action of the wires is changed from parallel to the longitudinal axis of the device to angles that are more appropriate to actuate the arms. In some embodiments small pulleys or bearing-like structures may be co-fabricated within the guides to reduce friction as the wires move. The force available to approximate the edges of the aperture is a function of several parameters, including the tension applied to the wires and the ratio of the length of the proximal arm section to that of the distal arm section. Thus, for example, the proximal arm section length can be a design parameter that is adjusted to achieve the required force.

A wire connector is shown in FIG. 144. The connector serves to couple the wire to the proximal end of the applier arm while allowing the arm to change its angle with respect to the wire as the arm rotates. The proximal end of the connector has an aperture for the wire, while the distal end has a hole (and release holes to allow pre-assembled co-fabrication of the connector and applier arm) that articulates with the pin in the groove at the proximal end of the applier arm. Slots near the proximal end of the connector allow access to the sides of the wire for purposes of attaching it to the connector through the application of an adhesive, solder, laser welding, etc. In other embodiments, the distal ends of the wires may be notch and retention clips located in the wire connector to engage the notches in the wires once the wires are inserted. In other embodiments, crimping or other deformation may be used to couple the wires and the connectors. FIGS. 145-146 show sectional views of the proximal end of the shaft along with portions of the right and left applier arms and wire connectors; the wires are illustrated, and their path through the shaft and termination within the connectors can be clearly seen. The wires are shown as rectangular in cross-section; however, wires with circular cross section would more commonly be used.

The proximal end of the shaft is shown in FIG. 147. The wires exit though the proximal apertures located within the tongue, such that the wires can pass through the lumen of the tubing.

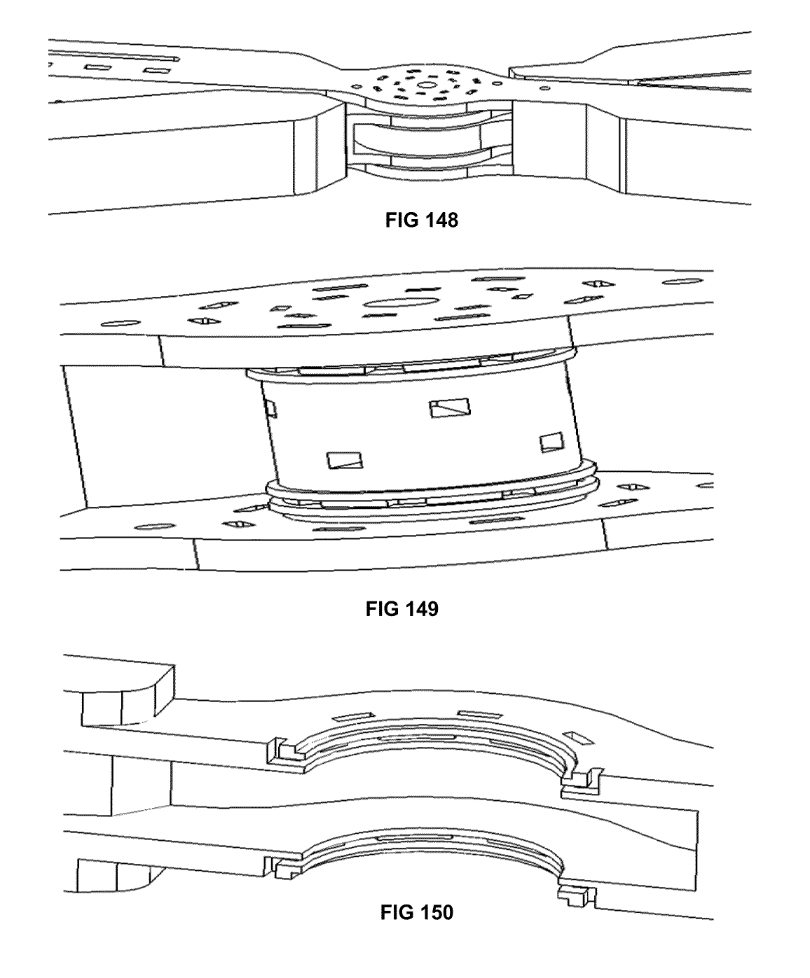

FIG. 148 shows the region of the device near the pivot. The pivot may be designed in a variety of ways; in the design shown in the figures, it is a type of box joint in which the arm support and left applier arm are perforated and rotate about a pin that is integral with the right applier arm. A box joint of this kind may readily be fabricated in a pre-assembled fashion using the multi-layer process previously described. However, in some embodiments, pivot designs that allow the two arms to be separately fabricated and then assembled may be used. To reduce clearances, the articulating surfaces of the right and left applier arms are studded with projections analogous to those of the slide and right applier arm channel described above. This stabilizes the pivot joint and avoids excessive misalignment of the half-clips when they are brought together. In the design shown, no projections are provided for the arm support, but these may be added in some embodiments.

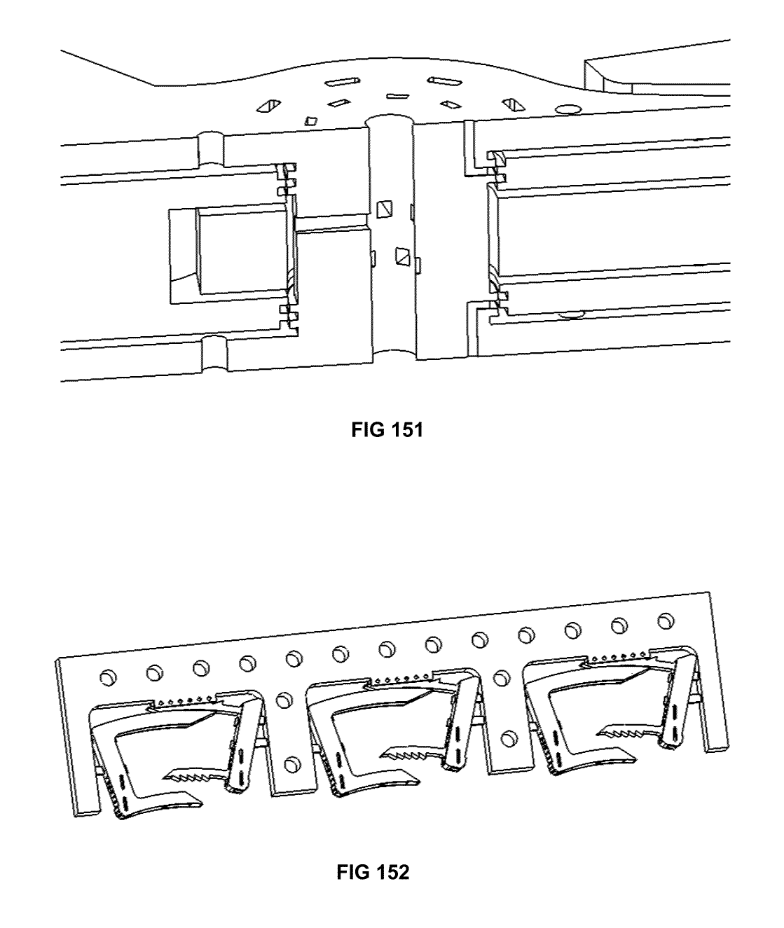

FIG. 149 shows the pin of the right applier arm, and FIG. 150 shows the perforations of the left applier arm. Release holes (including a large on-axis hole) are provided to allow removal of sacrificial material from the gaps between components, especially where projections reduce the effective size of these gaps. FIG. 151 shows the region of the pivot in cross section, with the two applier arms and arm support visible. The relationship between the projections on the right and left applier arms may be clearly seen.

As has been noted, the clip applier may be loaded or reloaded with half-clips that are fabricated separately. In some embodiments, reloading is performed outside the body, while in others, a magazine or other supply of half-clips may be introduced into the body for reloading there. FIG. 152 shows a group of three half-clip pairs in a frame, which is preferably co-fabricated with the half-clips. The half-clips are located so as to allow them to be loaded onto the holders of the clip applier when the slide is fully distal and the arms are separated by the angle that provides for the smallest cross-section (e.g., as shown in FIGS. 104 and 122. The half-clips are attached to the frame loosely by thin straps which pass through holes in the half-clips, and which are wider and/or higher than the holes adjacent to the surfaces of the half-clips, to provide lateral stability. Stops such as those shown in the figure may also be provided to restrain motion of the half-clips. Once the holders are slid into the half clips and the frame is held onto, the straps may be broken to release the half-clips by raising or lowering the clip applier out of its own (i.e., the horizontal) plane. Note that, while release holes are shown in the half-clips in FIG. 152, they are not required. The frame of FIG. 152 has the benefit of retaining half-clips without itself being higher (and thus adding cost in the multi-layer process disclosed above) than the half-clips themselves. In some embodiments, in lieu of the straps of FIG. 152, tabs may be used to hold the half-clips to a frame or plate; these are bent or break off when the half-clips are pulled away. In another embodiment, the half-clips are retained on a frame by incompletely-etched sacrificial material associated with the multi-layer fabrication process; after the holders have been inserted into the half-clips, the remaining sacrificial material is removed, freeing the half-clips.

In some embodiments, the prongs of the half-clips may be pivoted (and perhaps, spring-loaded like the wings of the device of the first group of embodiments), or formed from a superelastic material, such that they can be rotated more parallel to the bodies of the half-clips before delivery. If so rotated, then the cross-sectional area of the device can be further reduced, allowing a smaller access port, and since the prongs cannot interlock while rotated, the slide can be eliminated. In some embodiments, the prongs are rotated sufficiently parallel to the half-clip bodies that the overall cross section of the half-clip is small enough to be inserted within a channel in the clip applier arms. In this case, the clip may be delivered by pushing the half-clips distally until the prongs are free to extend while the applier holds onto the proximal ends of the half-clips, and then bringing the half-clips together so they interlock. In some embodiments, multiple half-clips may be loaded (e.g., in tandem—one behind the other) in the clip applier arms or in tubes that supply the distal ends of the arms, allowing multiple clips to be deployed in rapid succession.

An alternative embodiment to that shown in the figures may be used in which the distal prongs are shorter and/or of a different design (e.g., blunt) such that they do not penetrate the tissue (and thus do not interlock), but rather clamp it (the tissue edges may need to be everted proximally to allow this). In this case, stability of the clip must be provided entirely by the proximal prongs, which must be of a design (e.g., a 4-sided channel for the left half-clip) that prevents any undesired motion in or out of plane that could cause the half-clips to separate. In another embodiment, the distal prongs are located more proximally than shown in the figures, and the distal end of the clip is provided with shorter and/or differently-designed prongs or other projections intended to clamp tissue.

In some embodiments, the half-clips may be separated from each other after delivery of the clip by elastically or plastically deforming the bodies and/or prongs of one or both half-clips, such that the teeth are no longer engaged by the catch heads. For example, in the design shown in the figures, if the prongs of the left half-clip are spread further apart, or the prongs of the right half-clip are brought closer together, the half-clips may be separated. In some embodiments, features are provided (e.g., on the lateral surfaces of one or both half-clips) to facilitate deforming (e.g., by pinching with a tool) the clip-halves for purposes of removal.

In some embodiments, multiple devices may be ganged in a stack or other configuration such that multiple clips may be (though not necessarily are) delivered at the same time in a neighborhood of tissue (or to different neighborhoods or tissues). In this case, the orientation of all clips is not necessarily the same. Multiple devices of the kind associated with the first and second group of embodiments may also be deployed in a ganged fashion.

While the multi-layer fabrication process described above is a preferred—and in some cases, the only economically viable—way to manufacture the devices described herein, and the designs described assume this method of fabrication, other methods may be used. For example, the device of the third group of embodiments may be manufactured from standard shapes such as rods, tubes, and blocks, using conventional manufacturing techniques such as CNC machining, electrical discharge machining, and laser machining. Some key changes to the design shown may facilitate production by these methods. For example, a key function of the top and bottom of the left half-clip shown in FIG. 125 is to prevent the two half-clips from becoming disengaged from one another by sliding one vertically with respect to the other. Yet the geometry of the catch head and catch beam is difficult if not impossible to create with conventional manufacturing techniques due to limited access as a result of the top and bottom. Therefore, it may be necessary to delete the top and bottom in this case, and prevent vertical sliding by some other design feature. One example would be a ridge on the catch beams extending from the same side as the catch head; this can fit within a groove machined into the tooth side of the prongs of the right half-clip. Another example of design changes that may be made to accommodate conventional manufacturing is the slide. Using conventional manufacturing, this would generally be made as a separate piece of simpler that is inserted into a channel within the distal end of the right applier arm (which might be a tube of rectangular cross section). If the slide is provided with a tooth, then a hole drilled in the side of the channel would allow the slide catch head to reach the tooth so as to engage it. The slide catch beam could be laser-welded to the side of the applier arm. A further example of design changes would be the channel in the half-clips that accepts the half-clip holder. While it is difficult to conventionally manufacture a recess for the clip retention catch head in the style shown in FIG. 136, a hole drilled in the half-clip from the outside surface and extending into the channel (which may be formed by plunge EDM) to receive the catch head of the holder can serve a similar purpose.

Further Alternatives and Incorporations

To facilitate the delivery of the devices described herein, apparatus—either separate from the delivery system or incorporated into it—which provides means of temporarily holding tissue while it is being penetrated by needles or clip prongs and preventing it from moving away, may be provided. Such apparatus may include vacuum orifices, jaws, claws, or barbs, for example.

The various devices described herein may, as already noted with regard to the edge-to-edge cable embodiment, be used in multiples to approximate tissue, and optionally, a gradual tightening approach may be employed to reduce the pull-out stress on the tissue and/or allow a larger aperture to be closed.

In addition to the PFO closure application already described, the devices described herein have an unlimited variety of applications, not all of which are medical. Medical applications may include, for example:
Repair of mitral valve regurgitation using the edge-to-edge (double orifice) technique.
Closure of atrial and ventricular septal defects in the heart (particularly using the device of the first group of embodiments in conjunction with a patch). In this application, in order to close larger defects, multiple devices placed to span the defect may be tightened one at a time, approximating the defect edges without resorting to patches.
Anastomosis of blood vessels and other hollow structures, as well as solid structures such as nerve bundles.
Modifying the shape of the left ventricle to manage heart failure.
Surgery for morbid obesity in which plications are formed or devices are secured.
Surgery to correct gastroesophageal reflux disease.
Securing devices that might otherwise shift position, leak, etc., such as grafts used in the treatment of aortic aneurysms.
Closure of perforations in the stomach or other organs following endoluminal/natural orifice transluminal endoscopic surgery.
Fixation of tendons, cartilage, or other tissue to bone; for example, re-attachment of the meniscus in the knee joint.
Fixation of fractured bone fragments to one another.

Structural or sacrificial dielectric materials may be incorporated into embodiments of the present invention in a variety of different ways. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional

Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., and which is entitled “Electrochemical Fabrication Methods Incorporating Dielectric Materials and/or Using Dielectric Substrates”. These patent filings are each hereby incorporated herein by reference as if set forth in full herein.

Further teachings about planarizing layers and setting layers thicknesses and the like are set forth in the following U.S. patent applications which were filed Dec. 31, 2003: (1) U.S. Patent Application No. 60/534,159 by Cohen et al. and which is entitled “Electrochemical Fabrication Methods for Producing Multilayer Structures Including the use of Diamond Machining in the Planarization of Deposits of Material” and (2) U.S. Patent Application No. 60/534,183 by Cohen et al. and which is entitled “Method and Apparatus for Maintaining Parallelism of Layers and/or Achieving Desired Thicknesses of Layers During the Electrochemical Fabrication of Structures”. An additional filings providing teachings related to planarization are found in U.S. patent application Ser. No. 11/029,220, filed Jan. 3, 2005 by Frodis, et al., and which is entitled “Method and Apparatus for Maintaining Parallelism of Layers and/or Achieving Desired Thicknesses of Layers During the Electrochemical Fabrication of Structures”. These patent filings are each hereby incorporated herein by reference as if set forth in full herein.

The patent applications and patents set forth below are hereby incorporated by reference herein as if set forth in full. The teachings in these incorporated applications can be combined with the teachings of the instant application in many ways: For example, enhanced methods of producing structures may be derived from some combinations of teachings, enhanced structures may be obtainable, enhanced apparatus may be derived, and the like.

| US Pat App No, Filing Date | |
|---|---|
| US App Pub No, Pub Date | Inventor, Title |
| 09/493,496 - Jan. 28, 2000 | Cohen, “Method For Electrochemical Fabrication” |
| U.S. Pat. No. 6,790,377 - Sep. 14, 2004 | |
| 10/677,556 - Oct. 1, 2003 | Cohen, “Monolithic Structures Including Alignment and/or |
| 2004-0134772 - Jul. 15, 2004 | Retention Fixtures for Accepting Components” |
| 10/830,262 - Apr. 21, 2004 | Cohen, “Methods of Reducing Interlayer Discontinuities in |
| 2004-0251142A - Dec. 16, 2004 | Electrochemically Fabricated Three-Dimensional Structures” |
| U.S. Pat. No. 7,198,704 - Apr. 3, 2007 | |
| 10/271,574 -Oct. 15, 2002 | Cohen, “Methods of and Apparatus for Making High Aspect |
| 2003-0127336A - Jul. 10, 2003 | Ratio Microelectromechanical Structures” |
| U.S. Pat. No. 7,288,178 - Oct. 30, 2007 | |
| 10/697,597 - Dec. 20, 2002 | Lockard, “EFAB Methods and Apparatus Including Spray |
| 2004-0146650A - Jul. 29, 2004 | Metal or Powder Coating Processes” |
| 10/677,498 - Oct. 1, 2003 | Cohen, “Multi-cell Masks and Methods and Apparatus for |
| 2004-0134788 - Jul. 15, 2004 | Using Such Masks To Form Three-Dimensional Structures” |
| U.S. Pat. No. 7,235,166 - Jun. 26, 2007 | |
| 10/724,513 - Nov. 26, 2003 | Cohen, “Non-Conformable Masks and Methods and |
| 2004-0147124 - Jul. 29, 2004 | Apparatus for Forming Three-Dimensional Structures” |
| U.S. Pat. No. 7,368,044 - May 6, 2008 | |
| 10/607,931- Jun. 27, 2003 | Brown, “Miniature RF and Microwave Components and |
| 2004-0140862 - Jul. 22, 2004 | Methods for Fabricating Such Components” |
| U.S. Pat. No. 7,239,219 - Jul. 3, 2007 | |
| 10/841,100 - May 7, 2004 | Cohen, “Electrochemical Fabrication Methods Including Use |
| 2005-0032362 - Feb. 10, 2005 | of Surface Treatments to Reduce Overplating and/or |
| U.S. Pat. No. 7,109,118 - Sep. 19, 2006 | Planarization During Formation of Multi-layer Three- |
| | Dimensional Structures” |
| 10/387,958 - Mar. 13, 2003 | Cohen, “Electrochemical Fabrication Method and |
| 2003-022168A - Dec. 4, 2003 | Application for Producing Three-Dimensional Structures |
| | Having Improved Surface Finish” |
| 10/434,494 - May 7, 2003 | Zhang, “Methods and Apparatus for Monitoring Deposition |
| 2004-0000489A - Jan. 1, 2004 | Quality During Conformable Contact Mask Plating |
| | Operations” |
| 10/434,289 - May 7, 2003 | Zhang, “Conformable Contact Masking Methods and |
| 20040065555A - Apr. 8, 2004 | Apparatus Utilizing In Situ Cathodic Activation of a |
| | Substrate” |
| 10/434,294 - May 7, 2003 | Zhang, “Electrochemical Fabrication Methods With |
| 2004-0065550A - Apr. 8, 2004 | Enhanced Post Deposition Processing” |
| 10/434,295 - May 7, 2003 | Cohen, “Method of and Apparatus for Forming Three- |
| 2004-0004001A - Jan. 8, 2004 | Dimensional Structures Integral With Semiconductor Based |
| | Circuitry” |
| 10/434,315 - May 7, 2003 | Bang, “Methods of and Apparatus for Molding Structures |
| 2003-0234179 A - Dec. 25, 2003 | Using Sacrificial Metal Patterns” |
| U.S. Pat. No. 7,229,542 - Jun. 12, 2007 | |
| 10/434,103 - May 7, 2004 | Cohen, “Electrochemically Fabricated Hermetically Sealed |
| 2004-0020782A - Feb. 5, 2004 | Microstructures and Methods of and Apparatus for |
| U.S. Pat. No. 7,160,429 - Jan. 9, 2007 | Producing Such Structures” |
| 10/841,006 - May 7, 2004 | Thompson, “Electrochemically Fabricated Structures Having |
| 2005-0067292 - May 31, 2005 | Dielectric or Active Bases and Methods of and Apparatus for |
| | Producing Such Structures” |
| 10/434,519 - May 7, 2003 | Smalley, “Methods of and Apparatus for Electrochemically |
| 2004-0007470A - Jan. 15, 2004 | Fabricating Structures Via Interlaced Layers or Via Selective |
| U.S. Pat. No. 7,252,861 - Aug. 7, 2007 | Etching and Filling of Voids” |
| 10/724,515 - Nov. 26, 2003 | Cohen, “Method for Electrochemically Forming Structures |
| 2004-0182716 - Sep. 23, 2004 | Including Non-Parallel Mating of Contact Masks and |
| U.S. Pat. No. 7,291,254 - Nov. 6, 2007 | Substrates” |
| 10/841,347 - May 7, 2004 | Cohen, “Multi-step Release Method for Electrochemically |
| 2005-0072681 - Apr. 7, 2005 | Fabricated Structures” |
| 60/533,947 - Dec. 31, 2003 | Kumar, “Probe Arrays and Method for Making” |
| 10/841,300 - May 7, 2004 | Cohen, “Methods for Electrochemically Fabricating |
| 2005 0032375 - Feb. 10, 2005 | Structures Using Adhered Masks, Incorporating Dielectric |
| | Sheets, and/or Seed layers That Are Partially Removed Via |
| | Planarization” |
| 60/534,183 - Dec. 31, 2003 | Cohen, “Method and Apparatus for Maintaining Parallelism |
| | of Layers and/or Achieving Desired Thicknesses of Layers |
| | During the Electrochemical Fabrication of Structures” |
| 11/733,195 - Apr. 9, 2007 | Kumar, “Methods of Forming Three-Dimensional Structures |
| 2008-0050524 - Feb. 28, 2008 | Having Reduced Stress and/or Curvature” |
| 11/506,586 - Aug. 8, 2006 | Cohen, “Mesoscale and Microscale Device Fabrication |
| 2007-0039828 - Feb. 22, 2007 | Methods Using Split Structures and Alignment Elements” |
| U.S. Pat. No. 7,611,616 - Nov. 3, 2009 | |
| 10/949,744 - Sep. 24, 2004 | Lockard, “Three-Dimensional Structures Having Feature |
| 2005-0126916 - Jun. 16, 2005 | Sizes Smaller Than a Minimum Feature Size and Methods |
| U.S. Pat. No. 7,498,714 - Mar. 3, 2009 | for Fabricating” |

Various other embodiments of the present invention exist. Some of these embodiments may be based on a combination of the teachings herein with various teachings incorporated herein by reference. In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

We claim:

1. A medical instrument for approximating tissue within a patient's body during a minimally invasive surgical procedure, comprising:
(a) a first set of expandable elements;
(b) a second set of expandable elements;
(c) an elongated structure along which the first and second sets of expandable elements are located; and
(d) a locking mechanism for allowing the first and second sets of expandable elements to be moved to a more proximal position, relative to one another, while inhibiting movement of the first and second sets of expandable elements to a more distal positions relative to one another, along the length of the elongated element, after being moved to a more proximal position.

2. The medical instrument of claim 1 wherein the elongated structure comprises a rigid rail.

3. The medical instrument of claim 1 wherein the elongated structure comprises a rail that may bent along it length.

4. The medical instrument of claim 1 wherein the elongated structure comprises a chain.

5. The medical instrument of claim 1 wherein the elongated structure comprises a suture material.

6. The medical instrument of claim 1 wherein at least one of the first set of expandable elements or the second set of expandable elements comprise toggle wings that pivot open along an axis that is perpendicular to a longitudinal axis of the instrument.

7. The medical instrument of claim 6 wherein the toggle wings expand via a force induced by at least one spring located within the instrument.

8. The medical instrument of claim 7 wherein the other of the first set of expandable elements or the second set of expandable elements comprise toggle wings that pivot open along an axis that is perpendicular to a longitudinal axis of the instrument.

9. The medical instrument of claim 8 wherein the toggle wings of the other of the first set of expandable elements or the second set of expandable elements expand via a force induced by at least one spring located within the instrument.

10. The medical instrument of claim 1 wherein each of the first set and second set of expandable elements include toggle wings that pivot open along an axis that is perpendicular to a longitudinal axis of the instrument.

11. The medical instrument of claim 10 wherein the two sets of toggle elements one another when loaded in a delivery device.

12. The medical instrument of claim 10 wherein the two sets of toggle elements face in the same direction when loaded in a delivery device.

13. The medical instrument of claim 10 where after approximation, the two sets of expandable elements are on opposite sides of the approximated tissue.

14. The medical instrument of claim 10 where after approximation, the two sets of expandable elements are on the same side of the approximated tissue.

15. The medical instrument of claim 1 further comprising a curved delivery needle.

15. The medical instrument of claim 1 further comprising a straight delivery needle.

16. The medical instrument of claim 1 further comprising a needle having a slot extending from its delivery end.

17. The medical instrument of claim 1 wherein at least one of the first set of expandable elements or the second set of expandable elements comprise wings that expand by pivoting about an axis that is parallel to a longitudinal axis of the instrument are actuated via a rotational motion of the instrument along its longitudinal axis.

18. A surgical procedure for approximating tissue within a patient's body, comprising:
(a) locating an approximation instrument within the body of a patent at the end of a catheter; the instrument comprising:
(i) a first set of expandable elements located near a distal end of the instrument;
(ii) a second set of expandable elements located near a proximal end of the instrument;
(iii) an elongated element along which the first and second sets of expandable elements are located; and
(IV) a locking mechanism for allowing the first and second sets of expandable elements to be moved to a more proximal position while inhibiting movement of the first and second sets of expandable elements to a more distal position, along the length of the elongated element, after being moved to a more proximal position;
(b) inserting a distal end of the instrument through a proximal tissue region and then through a separated distal tissue region;
(c) expanding the first set of expandable elements and locating the elements against a wall of the distal tissue region;
(d) expanding the second set of expandable elements and locating the elements against a wall of the proximal tissue region;
(e) relatively moving the first set of expanded elements and the second set of expandable elements toward one another to bring the proximal and distal tissue regions into a more proximate position; and
(f) releasing at least a portion of the instrument from the catheter so that it remain in the body of the patient and retain the distal and proximal tissue regions in the more proximate position.

19. The procedure of claim 18 wherein the approximation instrument located at the end of the catheter comprises a plurality of approximation instruments that are deployable in sequence without removing the end of the catheter from the body of the patient.

20. The procedure of claim 18 wherein the multiple approximation instruments are located within a needle at the end of a catheter.

21. A medical instrument for approximating tissue within a patient's body during a minimally invasive surgical procedure, comprising:
(a) a first expandable element;
(b) a second expandable element;
(c) an elongated element along which the first and second expandable elements are located and separated one from the other;
(d) a mechanism for causing at least partial expansion of the first expandable element;
(e) a mechanism for causing at least partial expansion of the second expandable element; and
(f) a locking mechanism for allowing the first and second expandable elements to be moved to a more proximal position while inhibiting movement of the first and second sets of expandable elements to a more distal position, along the length of the elongated element, after being moved to a more proximal position.

22. The instrument of claim 21 which is fabricated from a plurality of layers of at least one structural material and at least one sacrificial material.

23. The instrument of claim 24 which is fabricated from a plurality of layers of at least one structural material and at least one sacrificial material.

24. A medical instrument for approximating two tissue elements within a patient's body during a minimally invasive surgical procedure, comprising:
(a) a first half of a clip
(b) a second half of the clip configured to engage the first half of the clip such that the two half when engaged can effectively hold two tissue elements in an approximated position;
(c) a first handle for holding the first half clip;
(d) a second handle for holding the second half clip;
(e) a pivotable element for holding the first and second handles in a desired position rotatable relative to one another and which upon rotation can be made to cause the first and second half clips to engage one another;
(f) a mechanism for separating the first and second half clips when engaged to one another from the first and second handles.
